(19) United States
(12) Patent Application Publication
(10) Pub. No.: US 2024/0262857 A1
(43) Pub. Date: Aug. 8, 2024

(54) NOVEL UNIVERSAL ANTI-RNA VIRUS AGENTS

(71) Applicant: Emory University, Atlanta, GA (US)

(72) Inventors: Raymond F. Schinazi, Miami, FL (US); Frank Amblard, Atlanta, GA (US)

(21) Appl. No.: 18/562,911

(22) PCT Filed: May 27, 2022
(86) PCT No.: PCT/US22/31381

§ 371 Date: Nov. 21, 2023

Related U.S. Application Data
(60) Provisional application No. 63/193,951, filed on May 27, 2021.

(51) Int. Cl. C07H 19/067; A61K 31/7068; A61K 45/06; A61P 31/14; C07B 59/00

(52) U.S. Cl. CPC C07H 19/067; A61K 31/7068; A61K 45/06; A61P 31/14; C07B 59/002; C07B 2200/05

(57) ABSTRACT

Deuterated and/or methylated N⁴-hydroxycytidine (NHC) analogs, with deuteration at one or both of the 2′- and 3′-positions on the ribo-sugar moiety and/or methylation of the 3′-positions on the ribo-sugar moiety, pharmaceutical compositions comprising one or more of these compounds, and, optionally, at least one additional therapeutic agent, and methods of treating or preventing infections caused by RNA viruses, curing an infection by an RNA virus, or reducing the biological activity of an RNA virus, are disclosed. Representative RNA viruses include, but are not limited to, Coronaviridae, such as MERSr-CoV, SARS-CoV-1, SARSCoV-2, HCoV-OC43, HCoV-229E, HCoV-NL63, and HCoV-HKU1, Picornaviridae, Hepeviridae, Noroviruses, Zika, Dengue, Mayaro, Influenza A and B, Parainfluenza, HCV, Rinovirus, tick-borne viruses, Ebola, Lassa, RSV, adenoviruses, enteroviruses, metapneumoviruses, Eastern, Western, and Venezuelan Equine Encephalitis (EEE, WEE and VEE, respectively), and Chikungunya fever (CHIK).

### FIELD OF THE INVENTION

This disclosure relates to N4-hydroxycytidine nucleoside and derivatives, as well as compositions and methods related thereto. In certain embodiments, the disclosure relates to the treatment or prophylaxis of infections caused by RNA viruses, such as Coronaviruses, Picornaviruses, Hepeviruses, Chikungunya fever (CHIK), Ebola, Influenza, RSV, Yellow Fever, Eastern, Western, and Venezuelan Equine Encephalitis (EEE, WEE and VEE, respectively), and Zika virus infections.

### BACKGROUND OF THE INVENTION

Ribonucleotide reductase (RNR) converts ribonucleotides to 2′-deoxyribonucleotides (at the 5′-diphosphate level), a reaction that is essential for DNA biosynthesis and repair. This enzyme is responsible for reducing all four ribonucleotide substrates. The purported mechanism for this reduction is shown below:

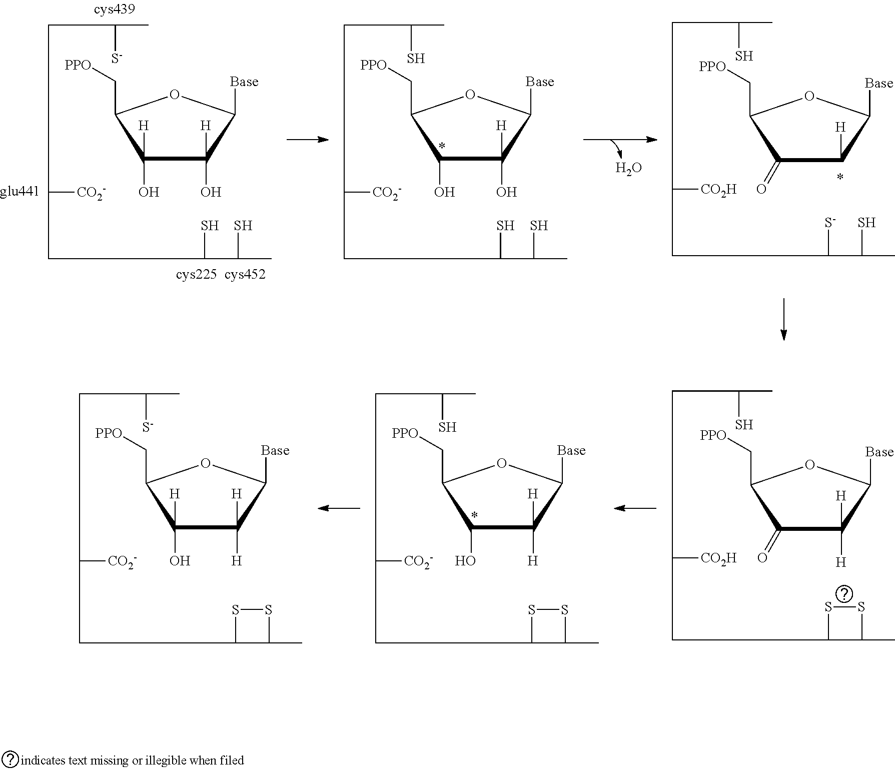

N⁴-hydroxycytidine (NHC) is an antiviral ribonucleoside analog that acts as a competitive alternative substrate for virally and cellular encoded RNA-dependent RNA polymerases. NHC has demonstrated broad in vitro antiviral activity, including SARS-CoV-2, Yellow fever virus, MERS-Cov, Zika virus, Venezuelan equine encephalitis virus, Chikungunya virus, Ebola, Norovirus, respiratory syncytial viruses, Influenza virus A & B and Hepatitis C (see, for example, Stuyver et al. (2003), Ribonucleotide analogue that blocks replication of bovine viral diarrhea and hepatitis C viruses in culture; Antimicrob. Agents Chemother., 47 244; Costantini et al. (2012), Antiviral activity of nucleoside analogues against norovirus; Antivir. Ther., 17 981; Reynard et al. (2015), Identification of a New Ribonucleoside Inhibitor of Ebola Virus Replication; Viruses, 7 6233; Ehteshami et al. (2017), Antimicrob. Agents Chemother., 61 e02395-16; Yoon et al. (2018), Orally efficacious broad-spectrum ribonucleoside analog inhibitor of influenza and respiratory syncytial viruses; Antimicrob. Agents Chemother., 62 e00766-18; Barnard et al. (2004), Inhibition of severe acute respiratory syndrome-associated coronavirus (SARSCoV) by calpain inhibitors and beta-D-N⁴-hydroxycytidine; Antivir. Chem. Chemother., 15 15; Pyrc et al. (2006), Inhibition of human coronavirus NL63 infection at early stages of the replication cycle; Antimicrob. Agents Chemother., 50 2000; Toots et al. (2019), Characterization of orally efficacious influenza drug with high resistance barrier in ferrets and human airway epithelia; Sci. Transl. Med., 11 eaax5866).

NHC has the ability to pair ambiguously as cytidine or uridine, thus introducing an elevated mutation load when incorporated into most RNA. Once in the ribonucleotide precursor pool, NHC can concentrate in the viral RNA genome over cellular RNA as viral RNA replication passes through the ribonucleotide pool multiple times for the synthesis of both plus and minus strands, resulting in lethal mutagenesis.

However, due to its mechanism of action, the mutagenic ribonucleoside analog NHC can be metabolized by the host cell to the 2′-deoxyribonucleotide-5′-diphosphate form (dNHC-DP) by ribonucleotide reductase, and then further phosphorylated to the rNHC-TP and then incorporated into DNA, leading to mutagenesis of the host (see, for example, Zhou et al., “β-D-N-4-hydroxycytidine (NHC) Inhibits SARS-CoV-2 Through Lethal Mutagenesis But Is Also Mutagenic To Mammalian Cells,” The Journal of Infectious Diseases, jiab247, https.//doi.org/10.1093/infdis/jiab247).

It would be advantageous to provide N⁴-hydroxycytidine analogs that are antiviral, but have less or no cellular mutagenicity. The present disclosure provides such analogs, and methods for their use in treating or preventing diseases caused by RNA viruses.

### SUMMARY OF THE INVENTION

In one embodiment, deuterated and/or methylated N⁴-hydroxycytidine (NHC) analogs, with deuteration and/or methylation at one or both of the 2′- and 3′-positions on the ribo-sugar moiety, are disclosed.

In another embodiment, pharmaceutical compositions comprising one or more compounds described herein, in combination with a pharmaceutically acceptable carrier or excipient, are also disclosed. In some aspects of this embodiment, the compositions comprise at least one compound described herein and at least one further therapeutic agent. This further therapeutic agent can be an antiviral compound, for example, one that functions via a different mechanism than the compounds described herein, or can exhibit other activity, such as anti-inflammatory activity, antibacterial activity, antithrombotic activity, and the like.

Methods for treating infections caused by RNA viruses, including, but not limited to, Coronaviridae, such as MERSr-CoV, SARS-CoV-1, SARS-CoV-2, HCoV-OC43, HCoV-229E, HCoV-NL63, and HCoV-HKU1, Picornaviridae, Hepeviridae, Noroviruses, Zika, Dengue, Mayaro, Influenza A and B, Parainfluenza, HCV, Rinovirus, tick-borne viruses, Ebola, Lassa, RSV, adenoviruses, enteroviruses, metapneumoviruses, Eastern, Western, and Venezuelan Equine Encephalitis (EEE, WEE and VEE, respectively), and Chikungunya fever (CHIK), are also disclosed.

The present invention will be better understood with reference to the following Detailed Description:

### BRIEF DESCRIPTION OF THE DRAWINGS

FIGS. 1A-D are charts showing the formation of 2′deoxy-NHC-DP and 2′deoxy-NHC-TP after reduction of NHC-DP by M1 ribonucleotide reductase (RNR), in terms of relative abundance (%) over time (min).

FIG. 2 is a chart showing the detection of 2′deoxy-NHC-TP after 50 μM NHC incubation in vero cells after 4 h at 37° C., in terms of relative abundance (%) over time (min).

FIG. 3 is a chart showing the results of a mammalian cell HPRT gene mutation assay: CHO-K1 cells (hamster cell line) were treated with drugs at the concentration of 10, 3 and 1 μM. After 6-thioguanine selection, resistant cell colonies were counted as a measure of HPRT knockout mutations in host cells. D-NHC reduces the number of colony formed by about 50% compared with NHC.

### DETAILED DESCRIPTION

Based on the mechanism shown above for the conversion of ribonucleotides to deoxyribonucleotides through ribonucleotide reductase (RNR), the present inventors discovered that single or multiple replacement of hydrogens with deuterium (carbon-hydrogen bonds to carbon-deuterium bond) or CH₃ at sites of metabolism in the sugar portion of NHC analogs (i.e., the 2′- and 3′-positions) slows down the rate of metabolism by preventing formation of key radical species. In turn, this provides the compounds with a relatively longer half-life than non-deuterated analogs, by slowing down or eliminating the metabolism of NHC to the mutagenic 2′-deoxy-NHC (dNHC). Thus, they can prevent partial or complete formation of mutagenic dNHC that can be incorporated to the host DNA

Deuterated and/or methylated NHC analogs, with deuteration at one or both of the 2′-and 3′-positions on the sugar moiety and/or methylation of the 3′-positions on the ribo-sugar moiety, pharmaceutical compositions comprising one or more compounds described herein, in combination with a pharmaceutically acceptable carrier or excipient, and methods of treating or preventing infections caused by RNA viruses are disclosed.

In certain embodiments, the compositions comprise at least one compound described herein and at least one further therapeutic agent. This further therapeutic agent can be an antiviral compound, for example, one that functions via a different mechanism than the compounds described herein, or can exhibit other activity, such as anti-inflammatory activity, antibacterial activity, antithrombotic activity, and the like.

The present invention will be better understood with reference to the following definitions:

### I. Definitions

The term “independently” is used herein to indicate that the variable, which is independently applied, varies independently from application to application. Thus, in a compound such as R″XYR″, wherein R″ is “independently carbon or nitrogen,” both R″ can be carbon, both R″ can be nitrogen, or one R″ can be carbon and the other R″ nitrogen.

As used herein, the term “enantiomerically pure” refers to a compound composition that comprises at least approximately 95%, and, preferably, approximately 97%, 98%, 99% or 100% of a single enantiomer of that compound.

As used herein, the term “substantially free of” or “substantially in the absence of” refers to a compound composition that includes at least 85 to 90% by weight, preferably 95% to 98% by weight, and, even more preferably, 99% to 100% by weight, of the designated enantiomer of that compound. In a preferred embodiment, the compounds described herein are substantially free of enantiomers.

Similarly, the term “isolated” refers to a compound composition that includes at least 85 to 90% by weight, preferably 95% to 98% by weight, and, even more preferably, 99% to 100% by weight, of the compound, the remainder comprising other chemical species or enantiomers.

The term “alkyl,” as used herein, unless otherwise specified, refers to a saturated straight, branched, or cyclic, primary, secondary, or tertiary hydrocarbons, including both substituted and unsubstituted alkyl groups. The alkyl group can be optionally substituted with any moiety that does not otherwise interfere with the reaction or that provides an improvement in the process, including but not limited to but limited to halo, haloalkyl, hydroxyl, carboxyl, acyl, aryl, acyloxy, amino, amido, carboxyl derivatives, alkylamino, dialkylamino, arylamino, alkoxy, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, acid halide, anhydride, oxime, hydrozine, carbamate, phosphonic acid, phosphonate, either unprotected, or protected as necessary, as known to those skilled in the art, for example, as taught in Greene, et al., Protective Groups in Organic Synthesis, John Wiley and Sons, Second Edition, 1991, hereby incorporated by reference. Specifically included are CF₃ and CH₂CF₃.

In the text, whenever the term C(alkyl range) is used, the term independently includes each member of that class as if specifically and separately set out. The term “alkyl” includes C₁₋₂₂ alkyl moieties, and the term “lower alkyl” includes C₁₋₆ alkyl moieties. It is understood to those of ordinary skill in the art that the relevant alkyl radical is named by replacing the suffix “-ane” with the suffix “-yl”.

As used herein, a “bridged alkyl” refers to a bicyclo- or tricyclo alkane, for example, a 2:1:1 bicyclohexane.

As used herein, a “spiro alkyl” refers to two rings that are attached at a single (quaternary) carbon atom.

The term “alkenyl” refers to an unsaturated, hydrocarbon radical, linear or branched, in so much as it contains one or more double bonds. The alkenyl group disclosed herein can be optionally substituted with any moiety that does not adversely affect the reaction process, including but not limited to but not limited to those described for substituents on alkyl moieties. Non-limiting examples of alkenyl groups include ethylene, methylethylene, isopropylidene, 1,2-ethane-diyl, 1,1-ethane-diyl, 1,3-propane-diyl, 1,2-propane-diyl, 1,3-butane-diyl, and 1,4-butane-diyl.

The term “alkynyl” refers to an unsaturated, acyclic hydrocarbon radical, linear or branched, in so much as it contains one or more triple bonds. The alkynyl group can be optionally substituted with any moiety that does not adversely affect the reaction process, including but not limited to those described above for alkyl moeities. Non-limiting examples of suitable alkynyl groups include ethynyl, propynyl, hydroxypropynyl, butyn-1-yl, butyn-2-yl, pentyn-1-yl, pentyn-2-yl, 4-methoxypentyn-2-yl, 3-methylbutyn-1-yl, hexyn-1-yl, hexyn-2-yl, and hexyn-3-yl, 3,3-dimethylbutyn-1-yl radicals.

The term “alkylamino” or “arylamino” refers to an amino group that has one or two alkyl or aryl substituents, respectively.

The term “fatty alcohol” as used herein refers to straight-chain primary alcohols with between 4 and 26 carbons in the chain, preferably between 8 and 26 carbons in the chain, and most preferably, between 10 and 22 carbons in the chain. The precise chain length varies with the source. Representative fatty alcohols include lauryl, stearyl, and oleyl alcohols. They are colourless oily liquids (for smaller carbon numbers) or waxy solids, although impure samples may appear yellow. Fatty alcohols usually have an even number of carbon atoms and a single alcohol group (—OH) attached to the terminal carbon. Some are unsaturated and some are branched. They are widely used in industry. As with fatty acids, they are often referred to generically by the number of carbon atoms in the molecule, such as “a C12 alcohol”, that is an alcohol having 12 carbons, for example dodecanol.

The term “protected” as used herein and unless otherwise defined refers to a group that is added to an oxygen, nitrogen, or phosphorus atom to prevent its further reaction or for other purposes. A wide variety of oxygen and nitrogen protecting groups are known to those skilled in the art of organic synthesis, and are described, for example, in Greene et al., Protective Groups in Organic Synthesis, supra.

The term “aryl”, alone or in combination, means a carbocyclic aromatic system containing one, two or three rings wherein such rings can be attached together in a pendent manner or can be fused. Non-limiting examples of aryl include phenyl, biphenyl, or naphthyl, or other aromatic groups that remain after the removal of a hydrogen from an aromatic ring. The term aryl includes both substituted and unsubstituted moieties. The aryl group can be optionally substituted with any moiety that does not adversely affect the process, including but not limited to but not limited to those described above for alkyl moieties. Non-limiting examples of substituted aryl include heteroarylamino, N-aryl-N-alkylamino, N-heteroarylamino-N-alkylamino, heteroaralkoxy, arylamino, aralkylamino, arylthio, monoarylamidosulfonyl, arylsulfonamido, diarylamidosulfonyl, monoaryl amidosulfonyl, arylsulfinyl, arylsulfonyl, heteroarylthio, heteroarylsulfinyl, heteroarylsulfonyl, aroyl, heteroaroyl, aralkanoyl, heteroaralkanoyl, hydroxyaralkyl, hydoxyheteroaralkyl, haloalkoxyalkyl, aryl, aralkyl, aryloxy, aralkoxy, aryloxyalkyl, saturated heterocyclyl, partially saturated heterocyclyl, heteroaryl, heteroaryloxy, heteroaryloxyalkyl, arylalkyl, heteroarylalkyl, arylalkenyl, and heteroarylalkenyl, carboaralkoxy.

The terms “alkaryl” or “alkylaryl” refer to an alkyl group with an aryl substituent. The terms “aralkyl” or “arylalkyl” refer to an aryl group with an alkyl substituent.

The term “halo,” as used herein, includes chloro, bromo, iodo and fluoro.

The term “acyl” refers to a carboxylic acid ester in which the non-carbonyl moiety of the ester group is selected from the group consisting of straight, branched, or cyclic alkyl or lower alkyl, alkoxyalkyl, including, but not limited to methoxymethyl, aralkyl, including, but not limited to, benzyl, aryloxyalkyl, such as phenoxymethyl, aryl, including, but not limited to, phenyl, optionally substituted with halogen (F, Cl, Br, or I), alkyl (including but not limited to C₁, C₂, C₃, and C₄) or alkoxy (including but not limited to C₁, C₂, C₃, and C₄), sulfonate esters such as alkyl or aralkyl sulphonyl including but not limited to methanesulfonyl, the mono, di or triphosphate ester, trityl or monomethoxytrityl, substituted benzyl, trialkylsilyl (e.g., dimethyl-t-butylsilyl) or diphenylmethylsilyl. Aryl groups in the esters optimally comprise a phenyl group. The term “lower acyl” refers to an acyl group in which the non-carbonyl moiety is lower alkyl.

The terms “alkoxy” and “alkoxyalkyl” embrace linear or branched oxy-containing radicals having alkyl moieties, such as methoxy radical. The term “alkoxyalkyl” also embraces alkyl radicals having one or more alkoxy radicals attached to the alkyl radical, that is, to form monoalkoxyalkyl and dialkoxyalkyl radicals. The “alkoxy” radicals can be further substituted with one or more halo atoms, such as fluoro, chloro or bromo, to provide “haloalkoxy” radicals. Examples of such radicals include fluoromethoxy, chloromethoxy, trifluoromethoxy, difluoromethoxy, trifluoroethoxy, fluoroethoxy, tetrafluoroethoxy, pentafluoroethoxy, and fluoropropoxy.

The term “alkylamino” denotes “monoalkylamino” and “dialkylamino” containing one or two alkyl radicals, respectively, attached to an amino radical. The terms arylamino denotes “monoarylamino” and “diarylamino” containing one or two aryl radicals, respectively, attached to an amino radical. The term “aralkylamino”, embraces aralkyl radicals attached to an amino radical. The term aralkylamino denotes “monoaralkylamino” and “diaralkylamino” containing one or two aralkyl radicals, respectively, attached to an amino radical. The term aralkylamino further denotes “monoaralkyl monoalkylamino” containing one aralkyl radical and one alkyl radical attached to an amino radical.

The term “heteroatom,” as used herein, refers to oxygen, sulfur, nitrogen and phosphorus.

The terms “heteroaryl” or “heteroaromatic,” as used herein, refer to an aromatic that includes at least one sulfur, oxygen, nitrogen or phosphorus in the aromatic ring.

The term “heterocyclic,” “heterocyclyl,” and cycloheteroalkyl refer to a nonaromatic cyclic group wherein there is at least one heteroatom, such as oxygen, sulfur, nitrogen, or phosphorus in the ring.

Nonlimiting examples of heteroaryl and heterocyclic groups include furyl, furanyl, pyridyl, pyrimidyl, thienyl, isothiazolyl, imidazolyl, tetrazolyl, pyrazinyl, benzofuranyl, benzothiophenyl, quinolyl, isoquinolyl, benzothienyl, isobenzofuryl, pyrazolyl, indolyl, isoindolyl, benzimidazolyl, purinyl, carbazolyl, oxazolyl, thiazolyl, isothiazolyl, 1,2,4-thiadiazolyl, isooxazolyl, pyrrolyl, quinazolinyl, cinnolinyl, phthalazinyl, xanthinyl, hypoxanthinyl, thiophene, furan, pyrrole, isopyrrole, pyrazole, imidazole, 1,2,3-triazole, 1,2,4-triazole, oxazole, isoxazole, thiazole, isothiazole, pyrimidine or pyridazine, and pteridinyl, aziridines, thiazole, isothiazole, 1,2,3-oxadiazole, thiazine, pyridine, pyrazine, piperazine, pyrrolidine, oxaziranes, phenazine, phenothiazine, morpholinyl, pyrazolyl, pyridazinyl, pyrazinyl, quinoxalinyl, xanthinyl, hypoxanthinyl, pteridinyl, 5-azacytidinyl, 5-azauracilyl, triazolopyridinyl, imidazolopyridinyl, pyrrolopyrimidinyl, pyrazolopyrimidinyl, adenine, N⁶-alkylpurines, N⁶-benzylpurine, N⁶-halopurine, N⁶-vinypurine, N⁶-acetylenic purine, N⁶-acyl purine,N⁶-hydroxyalkyl purine, N⁶-thioalkyl purine, thymine, cytosine, 6-azapyrimidine, 2-mercaptopyrmidine, uracil, N⁵-alkylpyrimidines, N⁵-benzylpyrimidines, N⁵-halopyrimidines, N⁵-vinylpyrimidine, N⁵-acetylenic pyrimidine, N⁵-acyl pyrimidine, N⁵-hydroxyalkyl purine, and N⁶-thioalkyl purine, and isoxazolyl. The heteroaromatic group can be optionally substituted as described above for aryl. The heterocyclic or heteroaromatic group can be optionally substituted with one or more substituents selected from the group consisting of halogen, haloalkyl, alkyl, alkoxy, hydroxy, carboxyl derivatives, amido, amino, alkylamino, and dialkylamino. The heteroaromatic can be partially or totally hydrogenated as desired. As a nonlimiting example, dihydropyridine can be used in place of pyridine. Functional oxygen and nitrogen groups on the heterocyclic or heteroaryl group can be protected as necessary or desired. Suitable protecting groups are well known to those skilled in the art, and include trimethylsilyl, dimethylhexylsilyl, t-butyldimethylsilyl, and t-butyldiphenylsilyl, trityl or substituted trityl, alkyl groups, acyl groups such as acetyl and propionyl, methanesulfonyl, and p-toluenelsulfonyl. The heterocyclic or heteroaromatic group can be substituted with any moiety that does not adversely affect the reaction, including but not limited to but not limited to those described above for aryl.

The term “host,” as used herein, refers to a unicellular or multicellular organism in which the virus can replicate, including but not limited to cell lines and animals, and, preferably, humans. Alternatively, the host can be carrying a part of the viral genome, whose replication or function can be altered by the compounds of the present invention. The term host specifically refers to infected cells, cells transfected with all or part of the viral genome and animals, in particular, primates (including but not limited to chimpanzees) and humans. In most animal applications of the present invention, the host is a human being. Veterinary applications, in certain indications, however, are clearly contemplated by the present invention (such as for use in treating chimpanzees).

The term nucleoside also includes ribonucleosides, and representative ribonucleosides are disclosed, for example, in the Journal of Medicinal Chemistry, 43(23), 4516-4525 (2000), Antimicrobial Agents and Chemotherapy, 45(5), 1539-1546 (2001), and PCT WO 2000069876.

The term “peptide” refers to a natural or synthetic compound containing two to one hundred amino acids linked by the carboxyl group of one amino acid to the amino group of another.

The term “pharmaceutically acceptable salt or prodrug” is used throughout the specification to describe any pharmaceutically acceptable form (such as an ester) compound which, upon administration to a patient, provides the compound. Pharmaceutically-acceptable salts include those derived from pharmaceutically acceptable inorganic or organic bases and acids. Suitable salts include those derived from alkali metals such as potassium and sodium, alkaline earth metals such as calcium and magnesium, among numerous other acids well known in the pharmaceutical art.

Pharmaceutically acceptable prodrugs refer to a compound that is metabolized, for example, hydrolyzed or oxidized, in the host to form the compound of the present invention. Typical examples of prodrugs include compounds that have biologically labile protecting groups on functional moieties of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. The prodrug forms of the compounds of this invention can possess antiviral activity, can be metabolized to form a compound that exhibits such activity, or both.

### II. Active Compounds

In one embodiment, the compounds have the following formula:

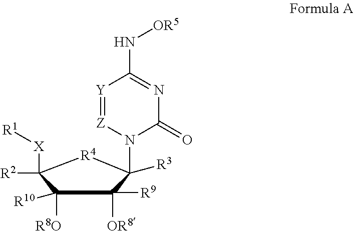

or a pharmaceutically acceptable salt or prodrug thereof, wherein:
X is CH₂, —CH(CH₃)—, CH(CH₃)₂—. CHF, CF₂ or CD₂,
Y is N or CR′,
Z is N or CR″,
R′ is H, deuterium or fluorine,
R″ is H, deuterium or methyl,
R¹ is OH, an optionally substituted O-linked amino acid, —O—C(O)—C₁₋₁₂ alkyl, —O—C(O)—C₂₋₁₂ alkenyl, —O—C(O)—C₂₋₁₂ alkynyl, —O—C(O)—C₃₋₆ cycloalkyl, —O—C(O)O—C₁₋₁₂ alkyl, —O—C(O)O—C₂₋₁₂ alkenyl, —O—C(O)O—C₂₋₁₂ alkynyl, —O—C(O)O—C₃₋₆ cycloalkyl, OC₁₋₆ alkyl, OC₁₋₆ haloalkyl, OC₁₋₆ alkoxy, OC₂₋₆ alkenyl, OC₂₋₆ alkynyl, OC₃₋₆ cycloalkyl, O—P(O)R⁶R⁷, O—CH₂—P—(OH)₃, O—CH₂—P—(OH)₃, or a mono-, di-, or triphosphate, wherein, when chirality exists at the phosphorous center of R⁴, it may be wholly or partially Rₚ or Sₚ or any mixture thereof,
R⁶ and R⁷ are independently selected from the group consisting of:
(a) OR¹⁵ where R¹⁵ selected from the group consisting of H,

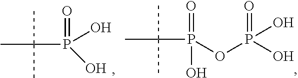

Li, Na, K, substituted or unsubstituted C₁₋₂₀ alkyl, substituted or unsubstituted C₃₋₆ cycloalkyl, C₁₋₄(alkyl)aryl, benzyl, C₁₋₆ haloalkyl, C₂₋₃(alkyl)OC₁₋₂₀alkyl, aryl, and heteroaryl, such as phenyl and pyridinyl, wherein aryl and heteroaryl are optionally substituted with zero to three substituents independently selected from the group consisting of (CH₂)₀₋₆CO₂R¹⁶ and (CH₂)₀₋₆ CON(R¹⁶)₂;
where R¹⁶ is independently H, substituted or unsubstituted C₁₋₂₀ alkyl, the carbon chain derived from a fatty alcohol or C₁₋₂₀ alkyl substituted with a C₁₋₆ alkyl, C₁₋₆ alkoxy, di(C₁₋₆ alkyl)-amino, fluoro, C₃₋₁₀ cycloalkyl, cycloalkyl-C₁₋₆ alkyl, cycloheteroalkyl, aryl, heteroaryl, substituted aryl, or substituted heteroaryl; wherein the substituents are C₁₋₅ alkyl, or C₁₋₅ alkyl substituted with a C₁₋₆ alkyl, alkoxy, di(C₁₋₆ alkyl)-amino, fluoro, C₃₋₁₀ cycloalkyl, or cycloalkyl;
   (b) the ester of a D- or L-amino acid

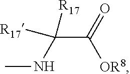

R¹⁷, R^{17′} and R¹⁸ are independently H, C₁₋₂₀ alkyl, the carbon chain derived from a fatty alcohol or C₁₋₂₀ alkyl optionally substituted with a C₁₋₆ alkyl, alkoxy, di(C₁₋₆alkyl)-amino, fluoro, C₃₋₁₀ cycloalkyl, cycloalkyl-C₁₋₆ alkyl, cycloheteroalkyl, aryl, heteroaryl, substituted aryl, or substituted heteroaryl; wherein the substituents are C₁₋₅ alkyl, or C₁₋₅ alkyl substituted with a C₁₋₆alkyl, alkoxy, di(C₁₋₆alkyl)-amino, fluoro, C₃₋₁₀ cycloalkyl, or cycloalkyl;
R² is H, deuterium, F, CN, N³, C₁₋₃ alkyl, C₂₋₃ alkynyl,
R³ is H, deuterium, CN, N³, C₁₋₃ alkyl, C₂₋₃ alkynyl,
R⁴ is O, CH₂, S, Se, CHF, CF₂, —C(CH₃)—, —C(cyclopropyl)-, C═CF₂ or C═CH₂,
R⁵ is H, an optionally substituted O-linked amino acid, —C(O)—C₁₋₁₂ alkyl, —C(O)—C₂₋₁₂ alkenyl, —C(O)—C₂₋₁₂ alkynyl, —C(O)—C₃₋₆ cycloalkyl, —C(O)O—C₁₋₁₂ alkyl, —C(O)O—C₂₋₁₂ alkenyl, —C(O)O—C₂₋₁₂ alkynyl, —C(O)O—C₃₋₆ cycloalkyl, wherein the groups can be substituted with one or more fluorine,
R⁸ and R^{8′} are independently selected from the group consisting of H, an optionally substituted O-linked amino acid, —C(O)—C₁₋₁₂ alkyl, —C(O)—C₂₋₁₂ alkenyl, —C(O)—C₂₋₁₂ alkynyl, —C(O)—C₃₋₆ cycloalkyl, —C(O)O—C₁₋₁₂ alkyl, —C(O)O—C₂₋₁₂ alkenyl, —C(O)O—C₂₋₁₂ alkynyl, —C(O)O—C₃₋₆ cycloalkyl, wherein the groups can be substituted with one or more substituents selected from the group consisting of fluorine, hydroxyl, amino, alkylamino, arylamino, and alkoxy,
R⁹ is H or deuterium, and
R¹⁰ is deuterium or methyl,
with the proviso that Y and Z are not both N.

In one aspect of this embodiment, X is —CH₂—.

In one aspect of this embodiment, one or both of Y and Z are CH.

In one aspect of this embodiment, R¹ is O—P(O)R⁶R⁷, and R⁵ and R⁶ are defined such that R¹ is a phosphoramidate.

In one aspect of this embodiment, R¹ is OH.

In one aspect of this embodiment, R¹ is —O—C(O)—C₁₋₁₂ alkyl.

In one aspect of this embodiment, R² is deuterium.

In one aspect of this embodiment, R³ is CN or N³.

In one aspect of this embodiment, R³ is H.

In one aspect of this embodiment, R⁴ is O.

In one aspect of this embodiment, R⁵ is H.

In one aspect of this embodiment, one or R⁶ and R⁷ is the ester of a D- or L-amino acid

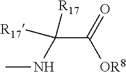

or O-aryl, and the other is O-aryl.

In one aspect of this embodiment, one or both of R⁸ and R⁹ are H.

In one aspect of this embodiment, R⁸ is —C(O)—C₁₋₁₂ alkyl.

In one aspect of this embodiment, R⁹ is D.

In one aspect of this embodiment, R¹⁰ is deuterium.

In one aspect of this embodiment, R¹⁰ is methyl.

These aspects can be present in any combination.

In another embodiment, the compounds have the following formula:

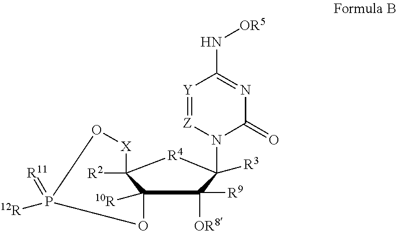

or a pharmaceutically acceptable salt or prodrug thereof, wherein:
X is CH₂, —CH(CH₃)—, CH(CH₃)₂—. CHF, CF₂ or CD₂,
Y is N or CR′,
Z is N or CR″,
R′ is H, deuterium or fluorine,
R″ is H, deuterium or methyl,
R² is H, deuterium, F, CN, N³, C₁₋₃ alkyl, or C₂₋₃ alkynyl,
R³ is H, deuterium, CN, N³, C₁₋₃ alkyl, or C₂₋₃ alkynyl,
R⁴ is O, CH₂, S, Se, CHF, CF₂, —C(CH₃)—, —C(cyclopropyl)-, C═CF₂ or C═CH₂,
R⁵ is H, an optionally substituted O-linked amino acid, —C(O)—C₁₋₁₂ alkyl, —C(O)—C₂₋₁₂ alkenyl, —C(O)—C₂₋₁₂ alkynyl, —C(O)—C₃₋₆ cycloalkyl, —C(O)O—C₁₋₁₂ alkyl, —C(O)O—C₂₋₁₂ alkenyl, —C(O)O—C₂₋₁₂ alkynyl, —C(O)O—C₃₋₆ cycloalkyl, wherein the groups can be substituted with one or more fluorine atoms,
R^{8′} is selected from the group consisting of H, an optionally substituted O-linked amino acid, —C(O)—C₁₋₁₂ alkyl, —C(O)—C₂₋₁₂ alkenyl, —C(O)—C₂₋₁₂ alkynyl, —C(O)—C₃₋₆ cycloalkyl, —C(O)O—C₁₋₁₂ alkyl, —C(O)O—C₂₋₁₂ alkenyl, —C(O)O—C₂₋₁₂ alkynyl, —C(O)O—C₃₋₆ cycloalkyl, wherein the groups can be substituted with one or more substituents selected from the group consisting of fluorine, hydroxyl, amino, alkylamino, arylamino, and alkoxy,
R⁹ is H or deuterium,
R¹⁰ is deuterium or methyl,
R¹ is 0 or S, and
R¹² is selected from the group consisting of:
(a) OR¹⁵ where R¹⁵ is selected from the group consisting of H, substituted or unsubstituted C₁₋₂₀alkyl, substituted or unsubstituted C₃₋₆cycloalkyl, C₁₋₄(alkyl)aryl, benzyl, C₁₋₆ haloalkyl, C₂₋₃(alkyl)OC₁₋₂₀ alkyl, aryl, and heteroaryl, such as phenyl and pyridinyl, wherein aryl and heteroaryl are optionally substituted with zero to three substituents independently selected from the group consisting of (CH₂)₀₋₆CO₂R¹⁶ and (CH₂)₀₋₆ CON(R¹⁶)₂;
(b) the ester of a D- or L-amino acid

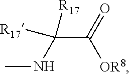

R¹⁷ and R¹⁸ are independently H, C₁₋₂₀ alkyl, the carbon chain derived from a fatty alcohol or C₁₋₂₀ alkyl optionally substituted with a C₁₋₆ alkyl, alkoxy, di(C₁₋₆alkyl)-amino, fluoro, C₃₋₁₀ cycloalkyl, cycloalkyl-C₁₋₆ alkyl, cycloheteroalkyl, aryl, heteroaryl, substituted aryl, or substituted heteroaryl; wherein the substituents are C₁₋₅ alkyl, or C₁₋₅ alkyl substituted with a C₁₋₆alkyl, alkoxy, di(C₁₋₆alkyl)-amino, fluoro, C₃₋₁₀ cycloalkyl, or cycloalkyl; and
(c)

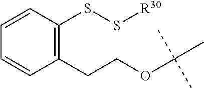

where R³⁰ is selected from the group consisting of substituted or unsubstituted C₁₋₂₀alkyl, substituted or unsubstituted C₃₋₆ cycloalkyl, substituted or unsubstituted (C₂₋₁₀)alkene, substituted or unsubstituted (C₂₋₁₀)alkyne, C₁₋₄(alkyl)aryl, aryl, heteroaryl, and C₁₋₆ haloalkyl, with the proviso that Y and Z cannot both be N.

In one aspect of this embodiment, X is —CH₂—.

In one aspect of this embodiment, one or both of Y and Z are CH.

In one aspect of this embodiment, R² is deuterium.

In one aspect of this embodiment, R³ is CN or N₃.

In one aspect of this embodiment, R³ is H.

In one aspect of this embodiment, R⁴ is O.

In one aspect of this embodiment, R⁵ is H.

In one aspect of this embodiment, R^{8′} is H.

In one aspect of this embodiment, R⁹ is D.

In one aspect of this embodiment, R¹⁰ is deuterium.

In one aspect of this embodiment, R¹⁰ is methyl.

In one aspect of this embodiment, R¹¹ is O.

In one aspect of this embodiment, R¹² is the ester of a D- or L-amino acid

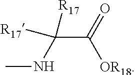

These aspects can be present in any combination.

In still another embodiment, the compounds have the following formula:

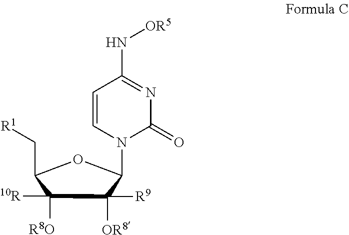

or a pharmaceutically acceptable salt or prodrug thereof, wherein:
R¹ is OH, an optionally substituted O-linked amino acid, —O—C(O)—C₁₋₁₂ alkyl, —O—C(O)—C₂₋₁₂ alkenyl, —O—C(O)—C₂₋₁₂ alkynyl, —O—C(O)—C₃₋₆ cycloalkyl, —O—C(O)O—C₁₋₁₂ alkyl, —O—C(O)O—C₂₋₁₂ alkenyl, —O—C(O)O—C₂₋₁₂ alkynyl, —O—C(O)O—C₃₋₆ cycloalkyl, OC₁₋₆ alkyl, OC₁₋₆ haloalkyl, OC₁₋₆ alkoxy, OC₂₋₆ alkenyl, OC₂₋₆ alkynyl, OC₃₋₆ cycloalkyl, O—P(O)R⁶R⁷, O—CH₂—P—(OH)₃, O—CH₂—P—(OH)₃, or a mono-, di-, or triphosphate, wherein, when chirality exists at the phosphorous center of R⁴, it may be wholly or partially Rₚ or Sₚ or any mixture thereof,
R⁶ and R⁷ are independently selected from the group consisting of:
(a) OR¹⁵ where R¹⁵ selected from the group consisting of H,

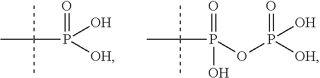

Li, Na, K, substituted or unsubstituted C₁₋₂₀alkyl, substituted or unsubstituted C₃₋₆cycloalkyl, C₁₋₄(alkyl)aryl, benzyl, C₁₋₆ haloalkyl, C₂₋₃(alkyl)OC₁₋₂₀alkyl, aryl, and heteroaryl, such as phenyl and pyridinyl, wherein aryl and heteroaryl are optionally substituted with zero to three substituents independently selected from the group consisting of (CH₂)₀₋₆CO₂R¹⁶ and (CH₂)₀₋₆ CON(R¹⁶)₂;
where R¹⁶ is independently H, substituted or unsubstituted C₁₋₂₀ alkyl, the carbon chain derived from a fatty alcohol or C₁₋₂₀ alkyl substituted with a C₁₋₆ alkyl, C₁₋₆ alkoxy, di(C₁₋₆ alkyl)-amino, fluoro, C₃₋₁₀ cycloalkyl, cycloalkyl-C₁₋₆ alkyl, cycloheteroalkyl, aryl, heteroaryl, substituted aryl, or substituted heteroaryl; wherein the substituents are C₁₋₅ alkyl, or C₁₋₅ alkyl substituted with a C₁₋₆ alkyl, alkoxy, di(C₁₋₆ alkyl)-amino, fluoro, C₃₋₁₀ cycloalkyl, or cycloalkyl;
   (b) the ester of a D- or L-amino acid

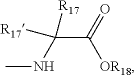

R¹⁷, R¹⁷ and R¹⁸ are independently H, C₁₋₂₀ alkyl, the carbon chain derived from a fatty alcohol or C₁₋₂₀ alkyl optionally substituted with a C₁₋₆ alkyl, alkoxy, di(C₁₋₆alkyl)-amino, fluoro, C₃₄₀ cycloalkyl, cycloalkyl-C₁₋₆ alkyl, cycloheteroalkyl, aryl, heteroaryl, substituted aryl, or substituted heteroaryl; wherein the substituents are C₁₋₅ alkyl, or C₁₋₅ alkyl substituted with a C₁₋₆alkyl, alkoxy, di(C₁₋₆alkyl)-amino, fluoro, C₃₋₁₀ cycloalkyl, or cycloalkyl;
R² is H, deuterium, F, CN, N³, C₁₋₃ alkyl, or C₂₋₃ alkynyl,
R⁵ is H, an optionally substituted O-linked amino acid, —C(O)—C₁₋₁₂ alkyl, —C(O)—C₂₋₁₂ alkenyl, —C(O)—C₂₋₁₂ alkynyl, —C(O)—C₃₋₆ cycloalkyl, —C(O)O—C₁₋₁₂ alkyl, —C(O)O—C₂₋₁₂ alkenyl, —C(O)O—C₂₋₁₂ alkynyl, —C(O)O—C₃₋₆ cycloalkyl, wherein the groups can be substituted with one or more fluorine atoms,
R⁸ and R^{8′} are independently selected from the group consisting of H, an optionally substituted O-linked amino acid, —C(O)—C₁₋₁₂ alkyl, —C(O)—C₂₋₁₂ alkenyl, —C(O)—C₂₋₁₂ alkynyl, —C(O)—C₃₋₆ cycloalkyl, —C(O)O—C₁₋₁₂ alkyl, —C(O)O—C₂₋₁₂ alkenyl, —C(O)O—C₂₋₁₂ alkynyl, —C(O)O—C₃₋₆ cycloalkyl, wherein the groups can be substituted with one or more substituents selected from the group consisting of fluorine, hydroxyl, amino, alkylamino, arylamino, and alkoxy,
R⁹ is H or deuterium, and
R¹⁰ is deuterium or methyl.

In one aspect of this embodiment, X is —CH₂—.

In one aspect of this embodiment, one or both of Y and Z are CH.

In one aspect of this embodiment, R¹ is O—P(O)R⁶R⁷, and R⁵ and R⁶ are defined such that R¹ is a phosphoramidate.

In one aspect of this embodiment, R¹ is OH.

In one aspect of this embodiment, R¹ is —O—C(O)—C₁₋₁₂ alkyl.

In one aspect of this embodiment, R⁵ is H.

In one aspect of this embodiment, one or R⁶ and R⁷ is the ester of a D- or L-amino acid

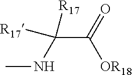

or O-aryl, and the other is O-aryl.

In one aspect of this embodiment, one or both of R⁸ and R^{8′} are H.

In one aspect of this embodiment, R⁸ is —C(O)—C₁₋₁₂ alkyl.

In one aspect of this embodiment, R⁹ is D.

In one aspect of this embodiment, R¹⁰ is deuterium.

In one aspect of this embodiment, R¹⁰ is methyl.

These aspects can be present in any combination.

In another embodiment, the compounds have the following formula:

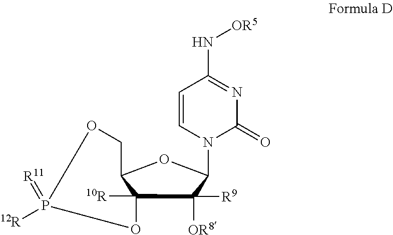

or a pharmaceutically acceptable salt or prodrug thereof, wherein:
R⁵ is H, an optionally substituted O-linked amino acid, —C(O)—C₁₋₁₂ alkyl, —C(O)—C₂₋₁₂ alkenyl, —C(O)—C₂₋₁₂ alkynyl, —C(O)—C₃₋₆ cycloalkyl, —C(O)O—C₁₋₁₂ alkyl, —C(O)O—C₂₋₁₂ alkenyl, —C(O)O—C₂₋₁₂ alkynyl, —C(O)O—C₃₋₆ cycloalkyl, wherein the groups can be substituted with one or more fluorine atoms,
R^{8′} is selected from the group consisting of H, an optionally substituted O-linked amino acid, —C(O)—C₁₋₁₂ alkyl, —C(O)—C₂₋₁₂ alkenyl, —C(O)—C₂₋₁₂ alkynyl, —C(O)—C₃₋₆ cycloalkyl, —C(O)O—C₁₋₁₂ alkyl, —C(O)O—C₂₋₁₂ alkenyl, —C(O)O—C₂₋₁₂ alkynyl, —C(O)O—C₃₋₆ cycloalkyl, wherein the groups can be substituted with one or more substituents selected from the group consisting of fluorine, hydroxyl, amino, alkylamino, arylamino, and alkoxy,
R⁹ is H or deuterium,
R¹⁰ is deuterium or methyl,
R¹¹ is O or S, and
R¹² is selected from the group consisting of:
(a) OR¹⁵ where R¹⁵ is selected from the group consisting of H, substituted or unsubstituted C₁₋₂₀alkyl, substituted or unsubstituted C₃₋₆cycloalkyl, C₁₋₄(alkyl)aryl, benzyl, C₁₋₆ haloalkyl, C₂₋₃(alkyl)OC₁₋₂₀ alkyl, aryl, and heteroaryl, such as phenyl and pyridinyl, wherein aryl and heteroaryl are optionally substituted with zero to three substituents independently selected from the group consisting of (CH₂)₀₋₆CO₂R¹⁶ and (CH₂)₀₋₆ CON(R¹⁶)₂;
(b) the ester of a D- or L-amino acid

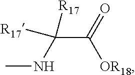

R⁷ and R¹⁸ are independently H, C₁₋₂₀ alkyl, the carbon chain derived from a fatty alcohol or C₁₋₂₀ alkyl optionally substituted with a C₁₋₆ alkyl, alkoxy, di(C₁₋₆alkyl)-amino, fluoro, C₃₋₁₀ cycloalkyl, cycloalkyl-C₁₋₆ alkyl, cycloheteroalkyl, aryl, heteroaryl, substituted aryl, or substituted heteroaryl; wherein the substituents are C₁₋₅ alkyl, or C₁₋₅ alkyl substituted with a C₁₋₆alkyl, alkoxy, di(C₁₋₆alkyl)-amino, fluoro, C₃₋₁₀ cycloalkyl, or cycloalkyl; and
(c)

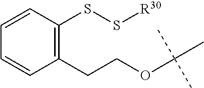

where R³⁰ is selected from the group consisting of substituted or unsubstituted C₁₋₂₀alkyl, substituted or unsubstituted C₃₋₆ cycloalkyl, substituted or unsubstituted (C₂₋₁₀)alkene, substituted or unsubstituted (C₂₋₁₀)alkyne, C₁₋₄(alkyl)aryl, aryl, heteroaryl, and C₁₋₆ haloalkyl.

In one aspect of this embodiment, R⁴ is O.

In one aspect of this embodiment, R⁵ is H.

In one aspect of this embodiment, R^{8′} is H.

In one aspect of this embodiment, R⁹ is D.

In one aspect of this embodiment, R¹⁰ is deuterium.

In one aspect of this embodiment, R¹⁰ is methyl.

In one aspect of this embodiment, R¹¹ is O.

In one aspect of this embodiment, R¹² is the ester of a D- or L-amino acid

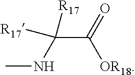

These aspects can be present in any combination.

In another embodiment, the compounds have the following formula:

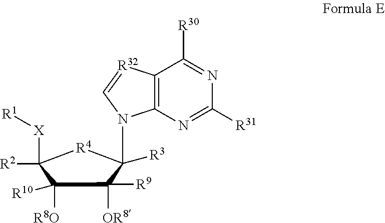

wherein:
R³⁰ is —NH—OH, NH—NH₂, —NH—OMe, —N(Me)-NH₂, or —NH—OR⁵,
R³¹ is H, F, or NH₂,
R³² is CH, CF or N,
and
R¹, R², R³, R⁴, R⁵, R⁶, R⁷, R⁸, R^{8′}, R⁹ and R¹⁰ are as defined in Formulas A and B.

In one aspect of this embodiment, R¹ is O—P(O)R⁶R⁷, and R⁵ and R⁶ are defined such that R¹ is a phosphoramidate.

In one aspect of this embodiment, R¹ is OH.

In one aspect of this embodiment, R¹ is —O—C(O)—C₁₋₁₂ alkyl.

In one aspect of this embodiment, R² is deuterium.

In one aspect of this embodiment, R³ is CN or N³.

In one aspect of this embodiment, R³ is H.

In one aspect of this embodiment, R⁴ is O.

In one aspect of this embodiment, R⁵ is H.

In one aspect of this embodiment, one or R⁶ and R⁷ is the ester of a D- or L-amino acid

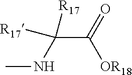

or O-aryl, and the other is O-aryl.

In one aspect of this embodiment, one or both of R⁸ and R^{8′} are H.

In one aspect of this embodiment, R⁸ is —C(O)—C₁₋₁₂ alkyl.

In one aspect of this embodiment, R⁹ is D.

In one aspect of this embodiment, R¹⁰ is deuterium.

In one aspect of this embodiment, R¹⁰ is methyl.

These aspects can be present in any combination.

In another embodiment, the compound has the formula:

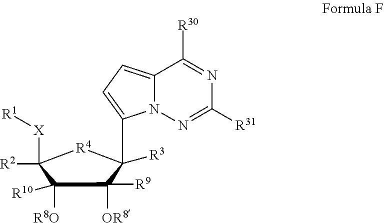

wherein:
R³⁰ is —NH—OH, NH—NH₂, —NH—OMe, —N(Me)-NH₂, or —NH—OR⁵,
R³¹ is H, F, or NH₂, and
R¹, R², R³, R⁴, R⁵, R⁶, R⁷, R⁸, R^{8′}, R⁹ and R¹⁰ are as defined in Formulas A and B.

In one aspect of this embodiment, R¹ is O—P(O)R⁶R⁷, and R⁵ and R⁶ are defined such that R¹ is a phosphoramidate.

In one aspect of this embodiment, R¹ is OH.

In one aspect of this embodiment, R¹ is —O—C(O)—C₁₋₁₂ alkyl.

In one aspect of this embodiment, R² is deuterium.

In one aspect of this embodiment, R³ is CN or N³.

In one aspect of this embodiment, R³ is H.

In one aspect of this embodiment, R⁴ is O.

In one aspect of this embodiment, R⁵ is H.

In one aspect of this embodiment, one or R⁶ and R⁷ is the ester of a D- or L-amino acid

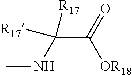

or O-aryl, and the other is O-aryl.

In one aspect of this embodiment, one or both of R⁸ and R^{8′} are H.

In one aspect of this embodiment, R⁸ is —C(O)—C₁₋₁₂ alkyl.

In one aspect of this embodiment, R⁹ is D.

In one aspect of this embodiment, R¹⁰ is deuterium.

In one aspect of this embodiment, R¹⁰ is methyl.

These aspects can be present in any combination.

In another embodiment, the compound has the following formula:

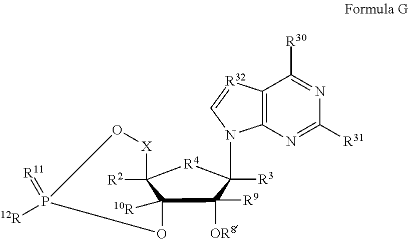

wherein:
R³⁰ is —NH—OH, NH—NH₂, —NH—OMe, —N(Me)-NH₂, or —NH—OR⁵,
R³¹ is H, F, or NH₂,
R³² is CH, CF or N,
and
R², R³, R⁴, R⁵, R^{8′}, R⁹, R¹⁰, R¹¹ and R¹² are as defined in Formulas A and B.

In one aspect of this embodiment, R² is deuterium.

In one aspect of this embodiment, R³ is CN or N³.

In one aspect of this embodiment, R³ is H.

In one aspect of this embodiment, R⁴ is O.

In one aspect of this embodiment, R⁵ is H.

In one aspect of this embodiment, R^{8′} is H.

In one aspect of this embodiment, R⁹ is deuterium.

In one aspect of this embodiment, R¹⁰ is deuterium.

In one aspect of this embodiment, R¹⁰ is methyl.

In one aspect of this embodiment, R¹¹ is O.

In one aspect of this embodiment, R¹² is the ester of a D- or L-amino acid

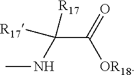

These aspects can be present in any combination.

In another embodiment, the compound has the following formula:

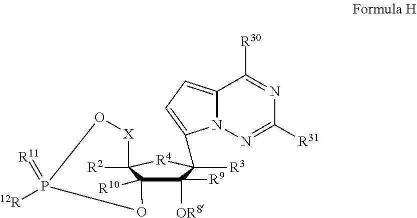

wherein:
R³⁰ is —NH—OH, NH—NH₂, —NH—OMe, —N(Me)-NH₂, or —NH—OR⁵,
R³¹ is H, F, or NH₂, and
R², R³, R⁴, R⁵, R^{8′}, R⁹, R¹⁰, R¹¹ and R¹² are as defined in Formulas A and B.

In one aspect of this embodiment, R² is deuterium.

In one aspect of this embodiment, R³ is CN or N³.

In one aspect of this embodiment, R³ is H.

In one aspect of this embodiment, R⁴ is O.

In one aspect of this embodiment, R⁵ is H.

In one aspect of this embodiment, R^{8′} is H.

In one aspect of this embodiment, R⁹ is deuterium.

In one aspect of this embodiment, R¹⁰ is deuterium.

In one aspect of this embodiment, R¹⁰ is methyl.

In one aspect of this embodiment, R¹¹ is O.

In one aspect of this embodiment, R¹² is the ester of a D- or L-amino acid

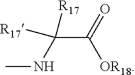

These aspects can be present in any combination.

In one embodiment, the compound has one of the following formulas:

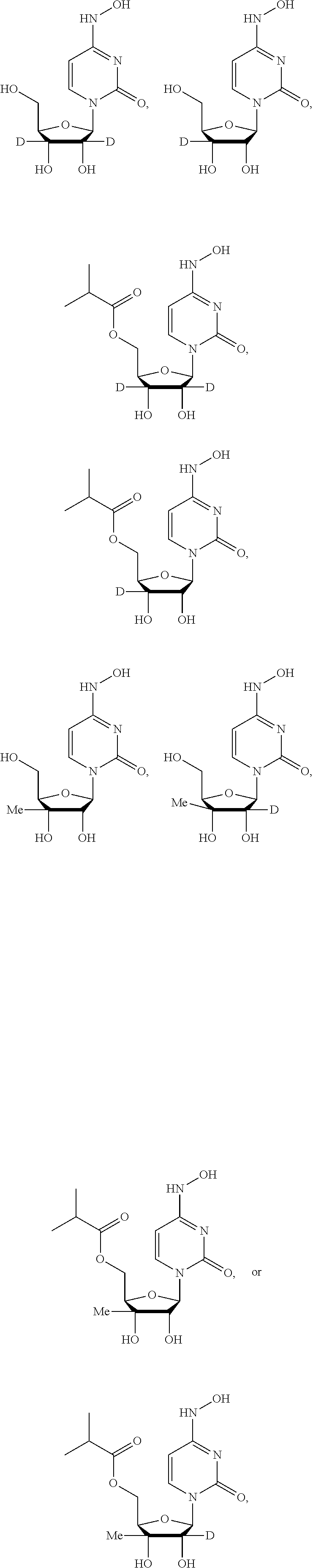

or a pharmaceutically acceptable salt or prodrug thereof.

In another embodiment, the compound has one of the following formulas:

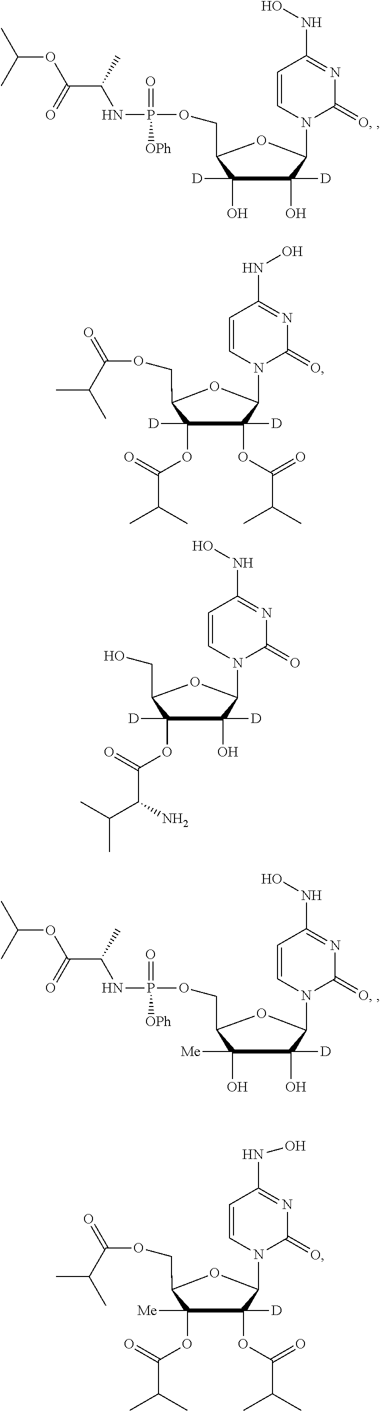

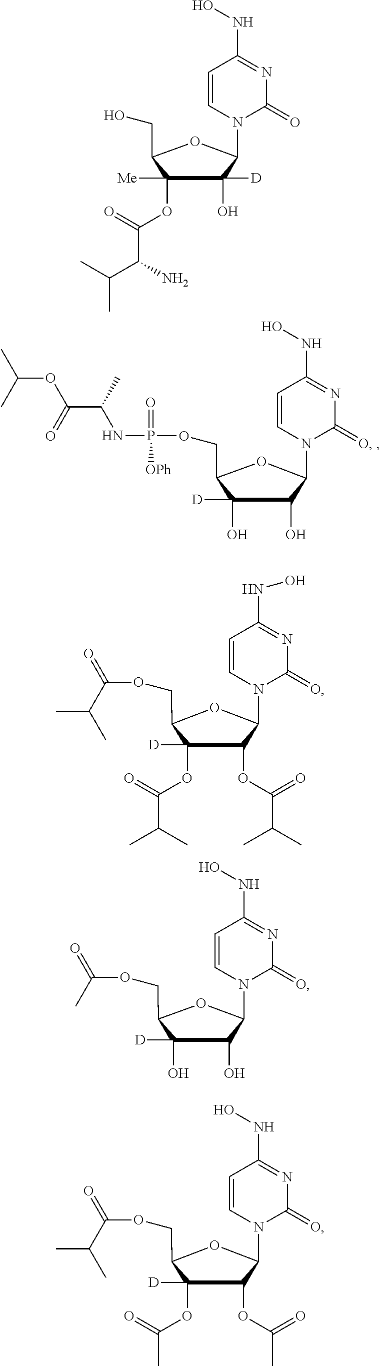

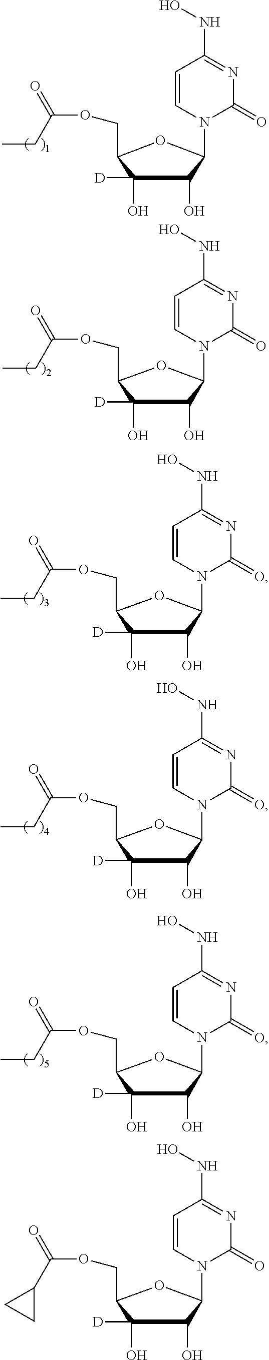

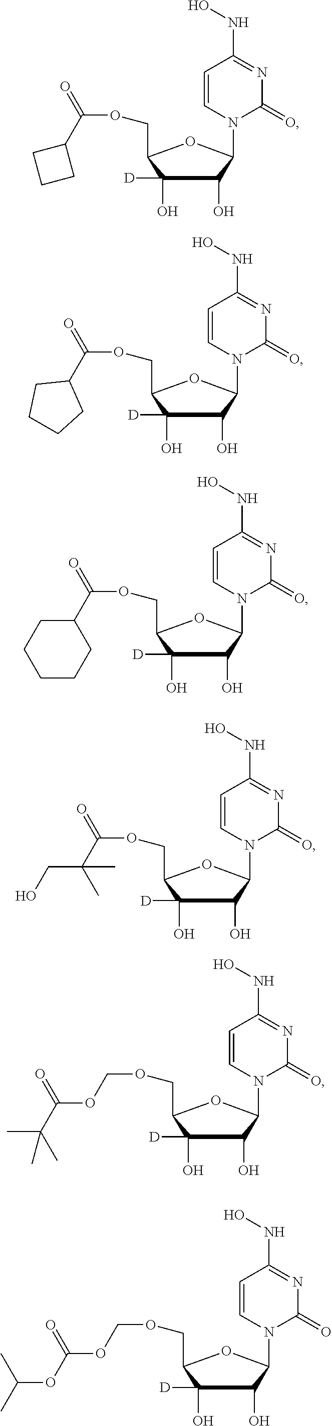

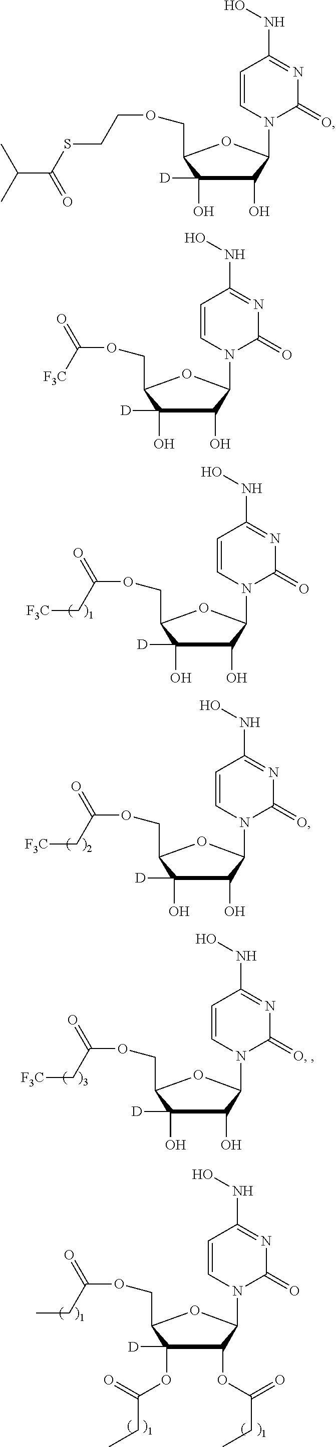

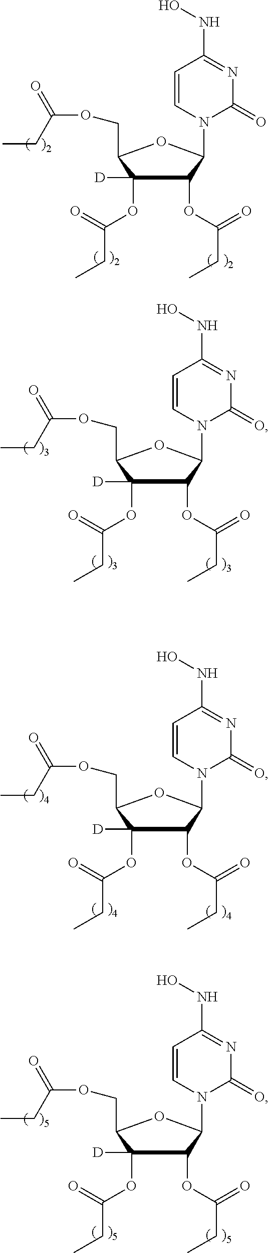

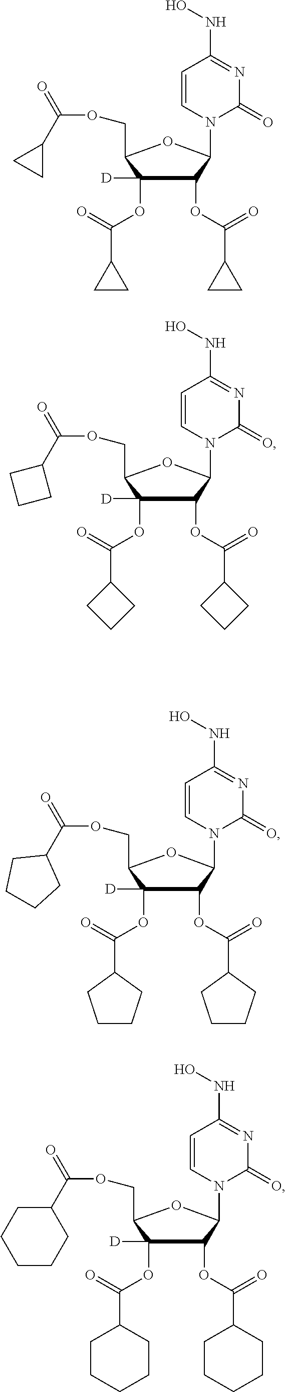

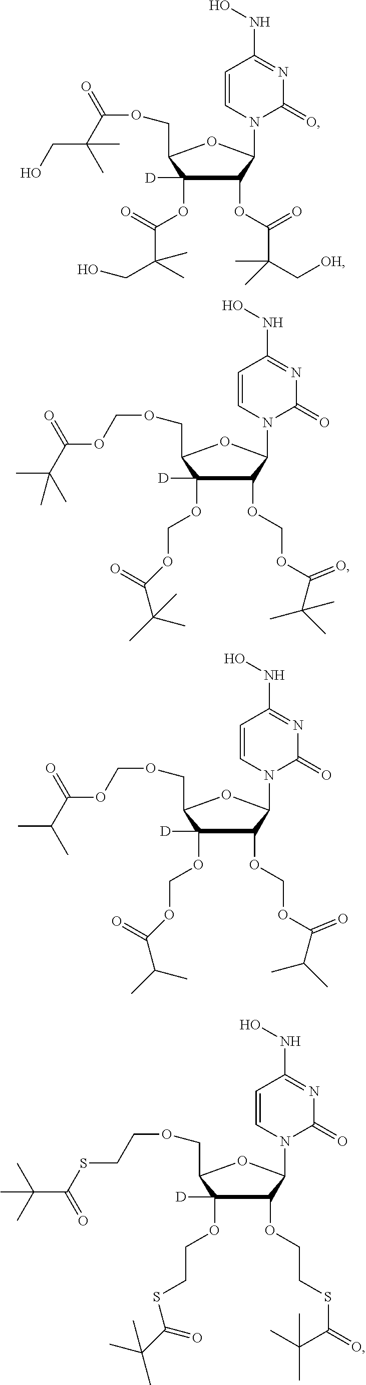

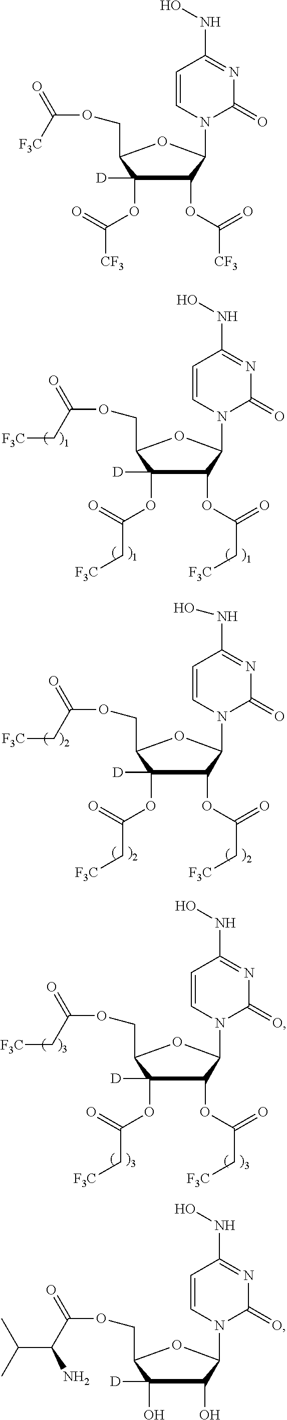

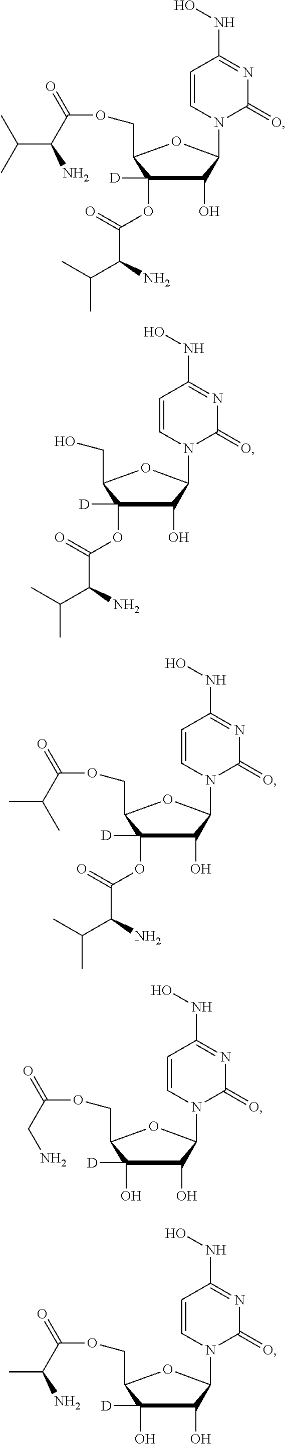

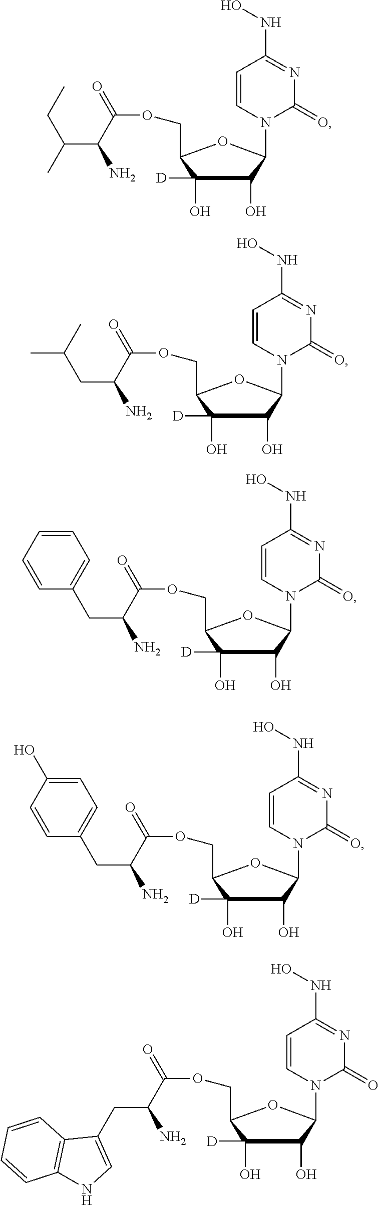

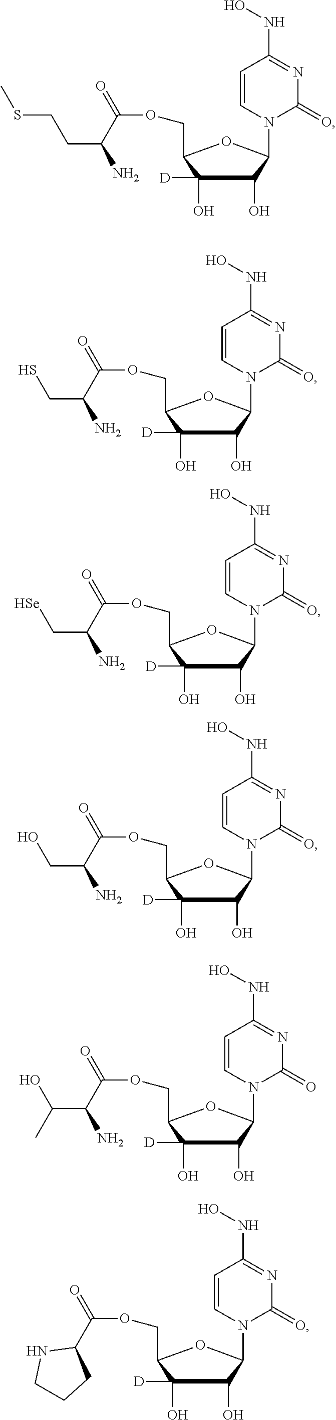

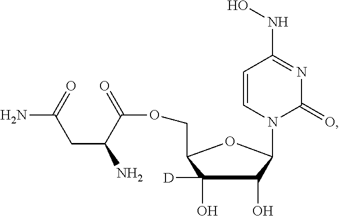

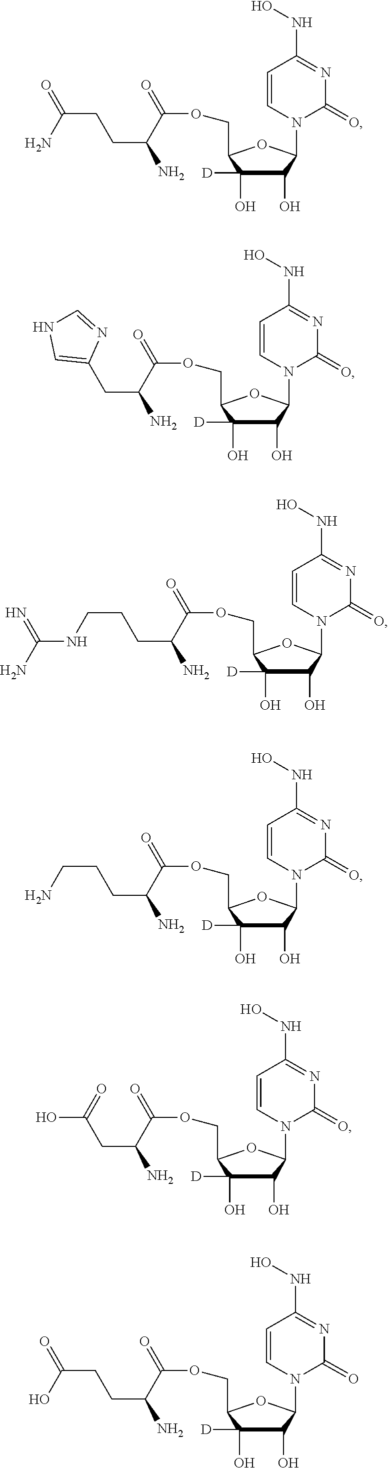

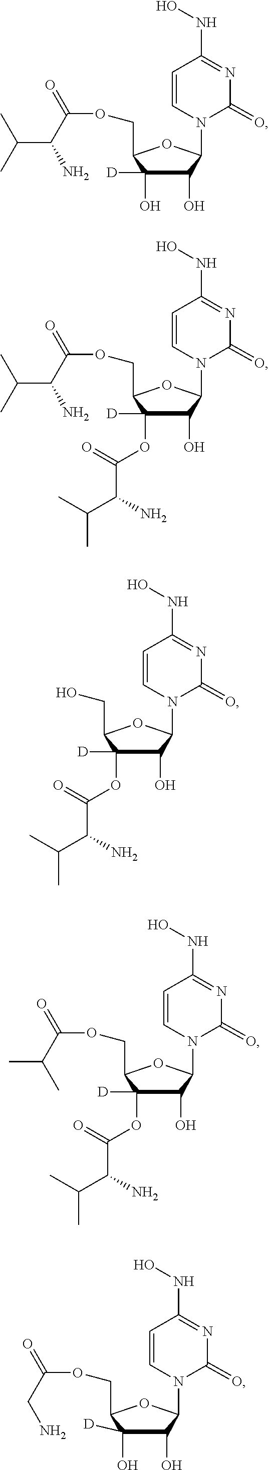

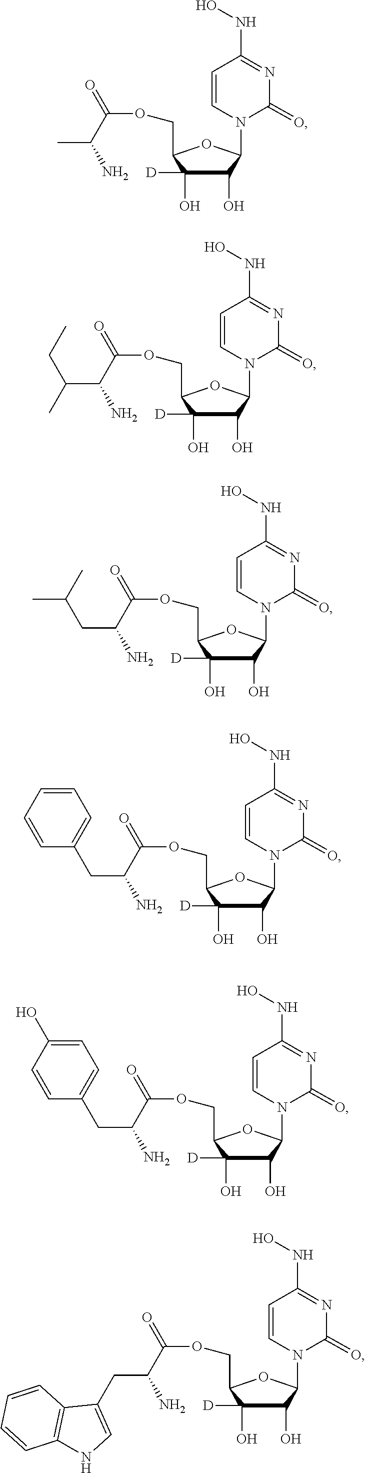

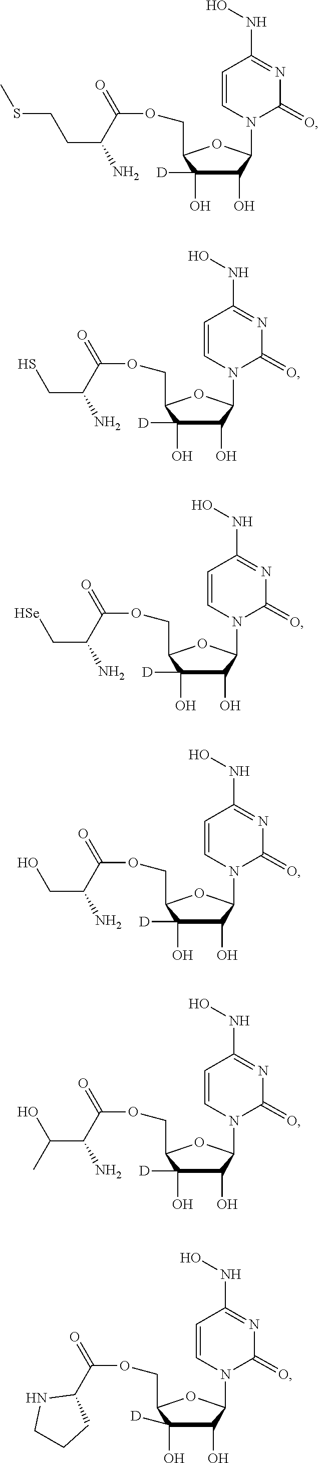

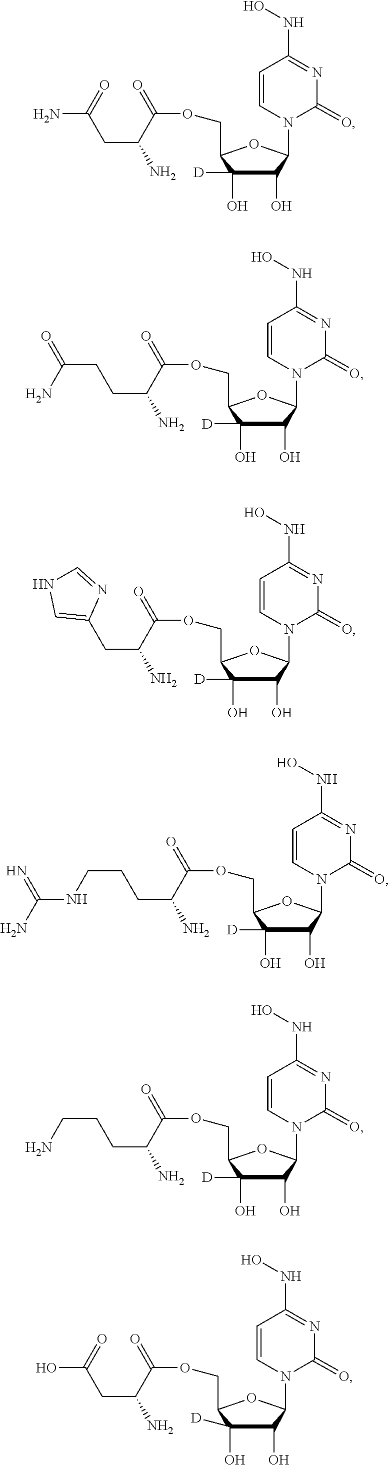

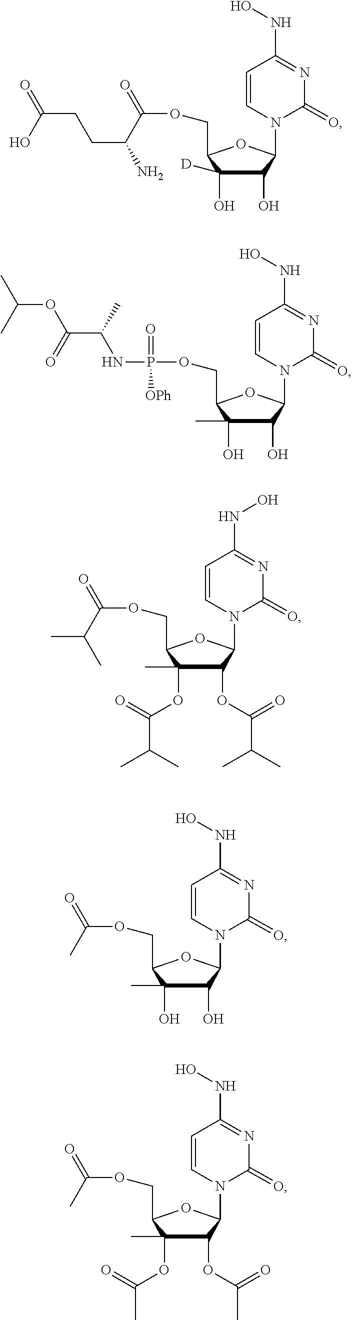

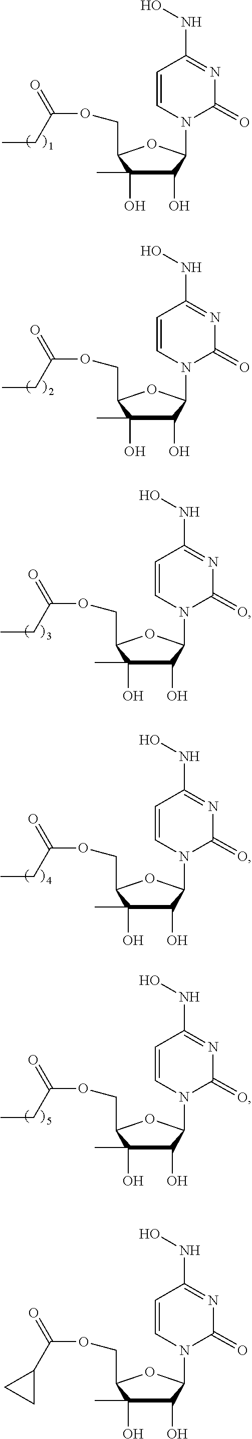

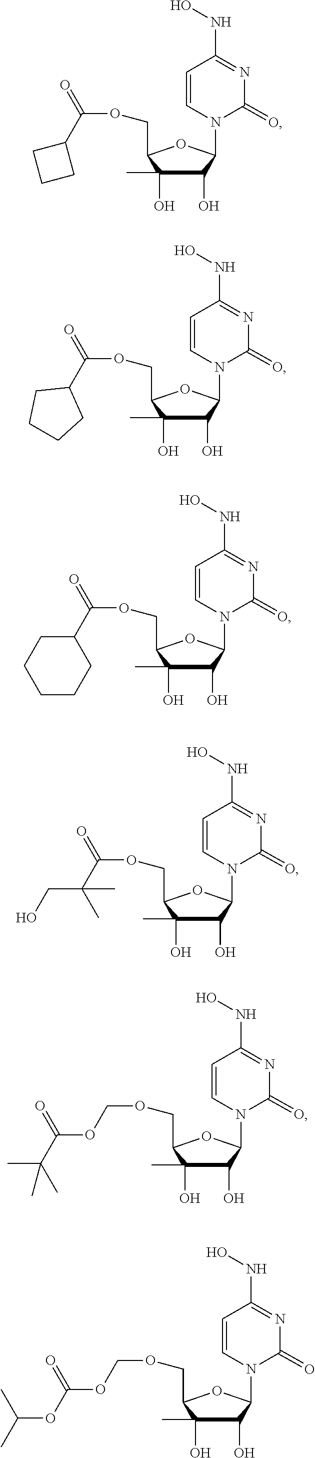

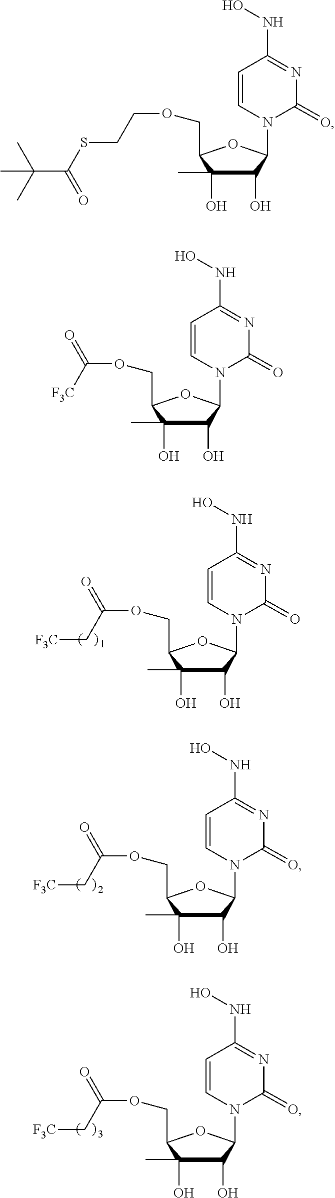

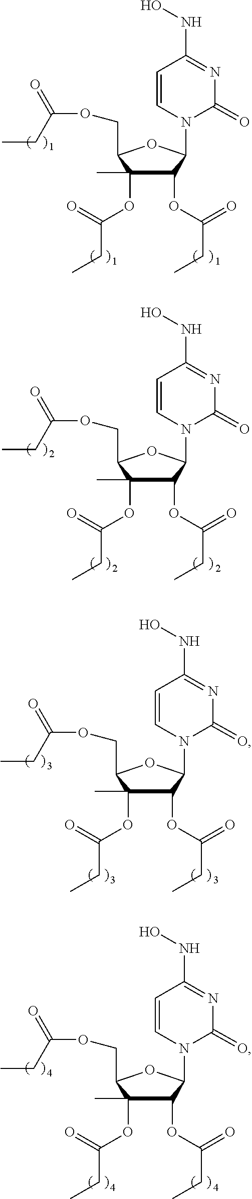

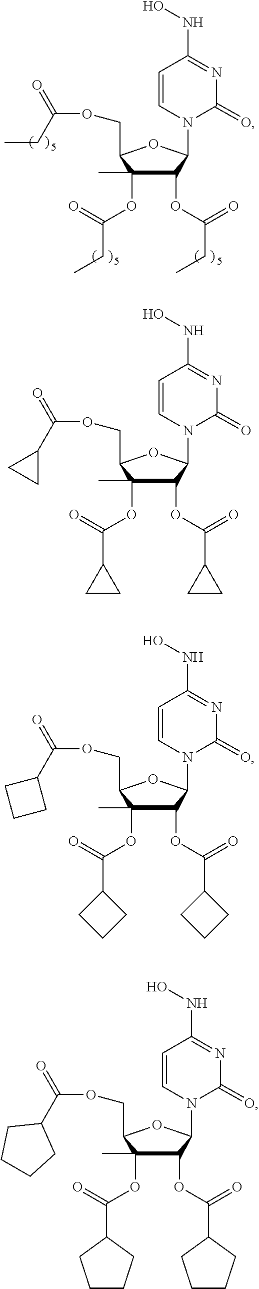

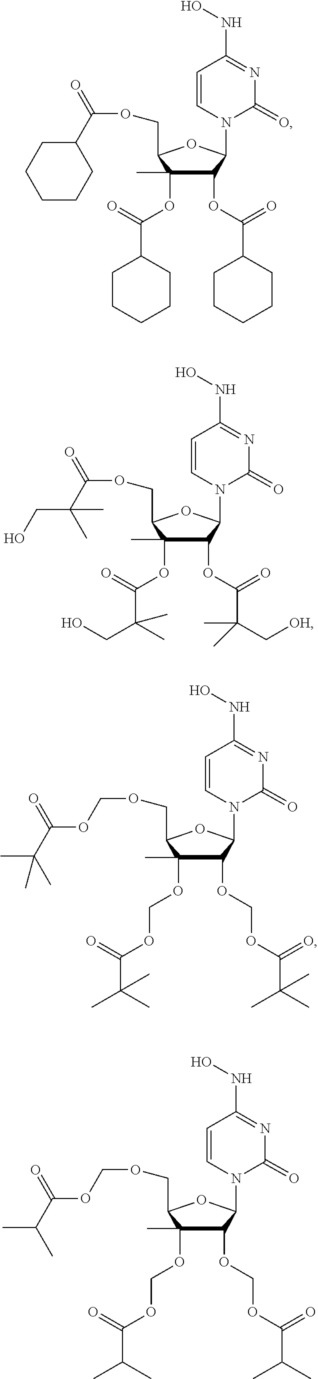

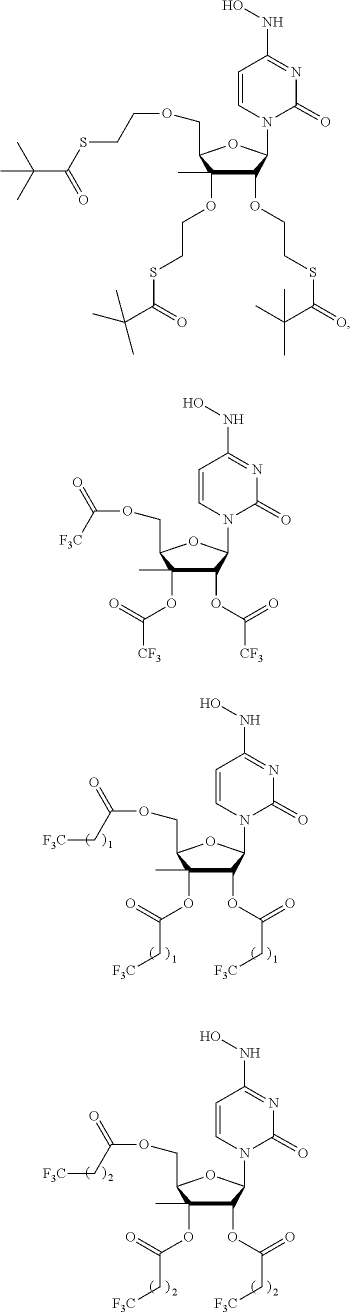

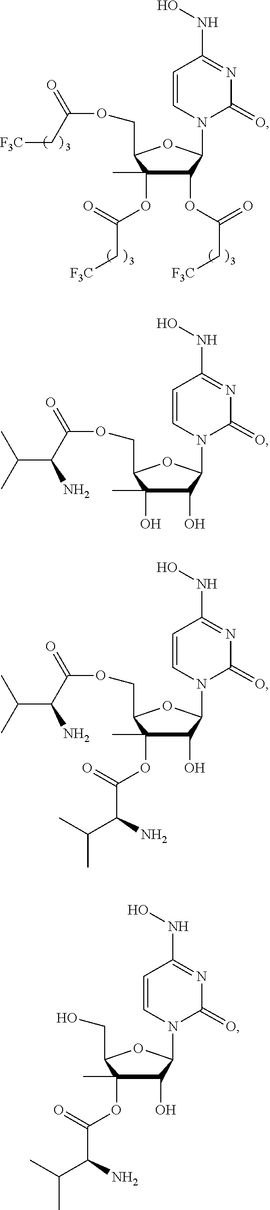

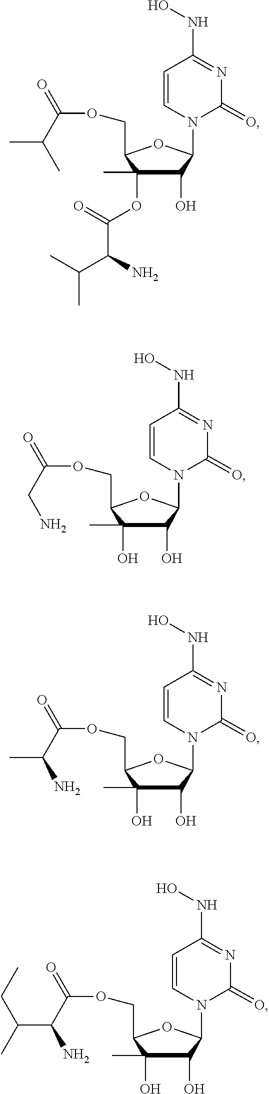

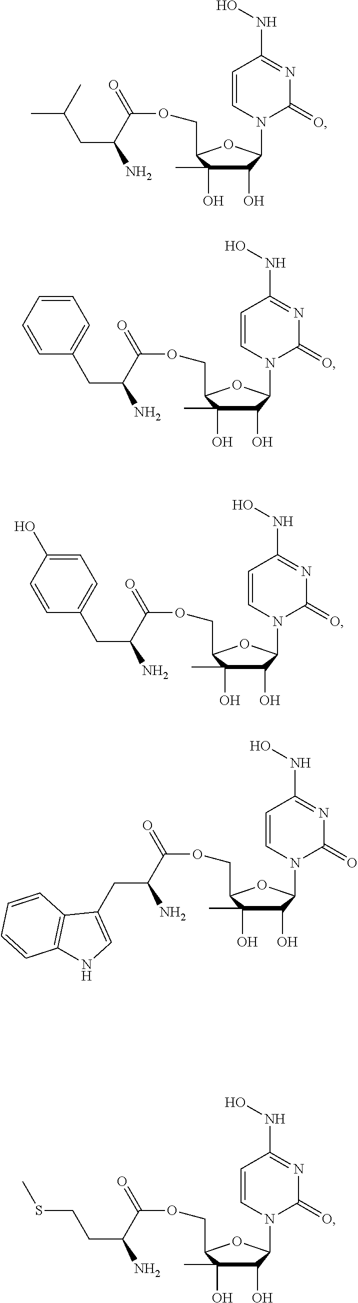

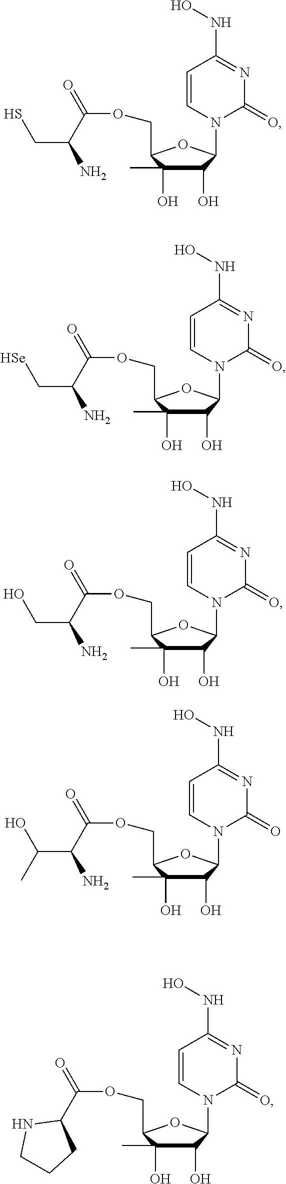

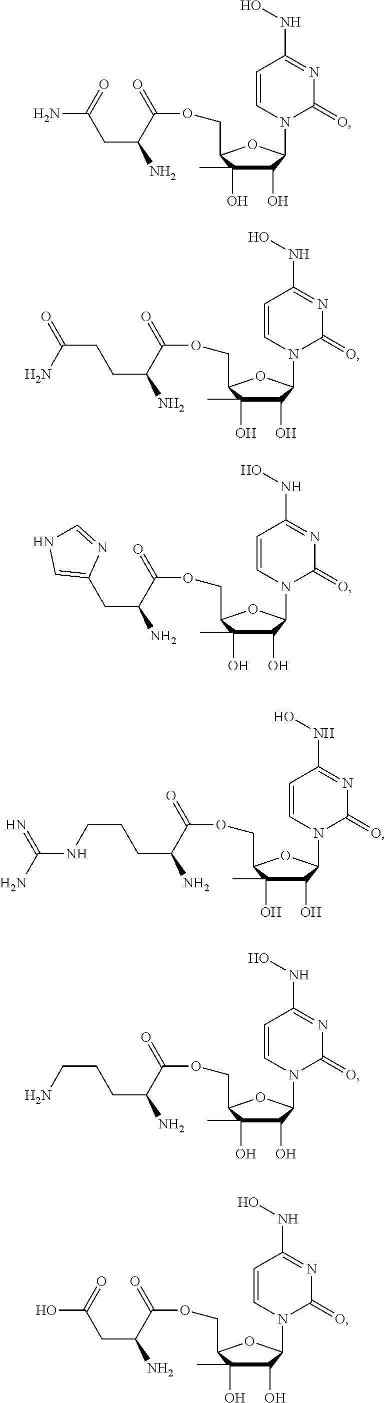

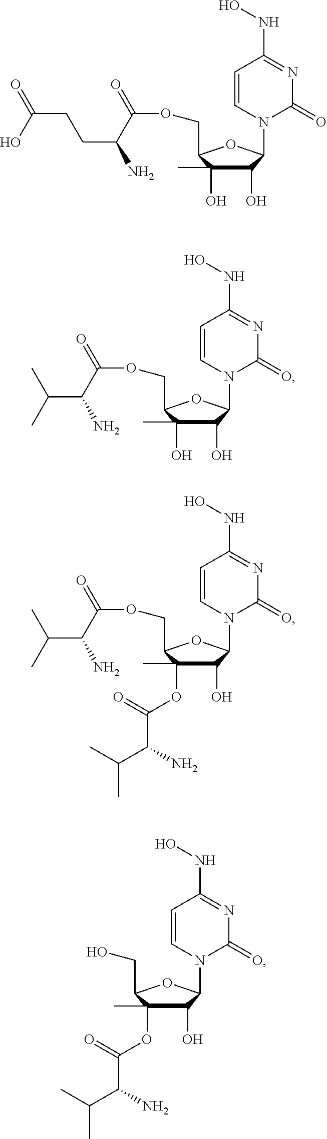

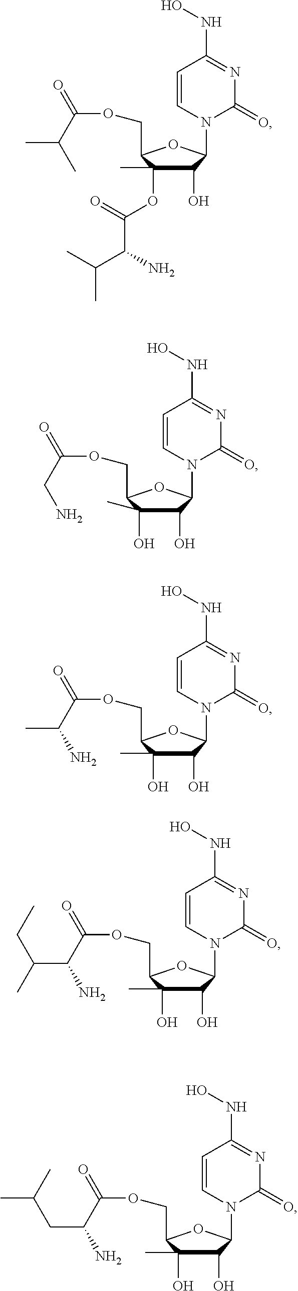

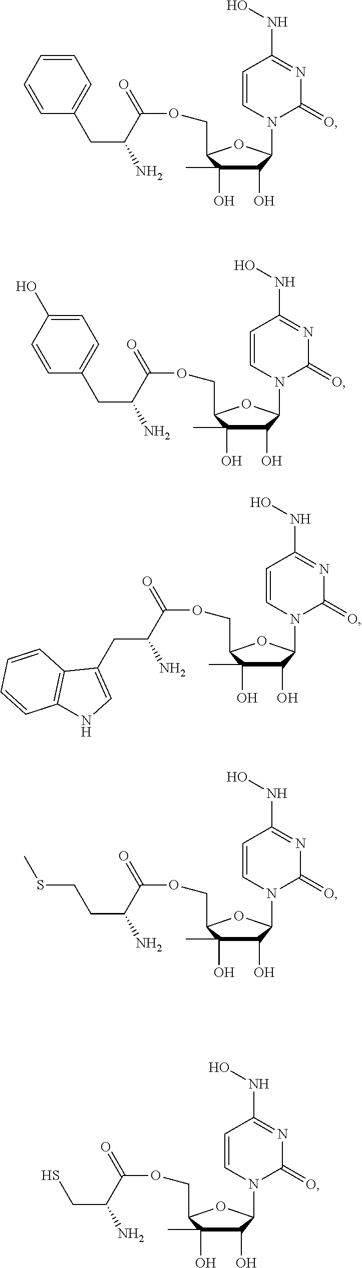

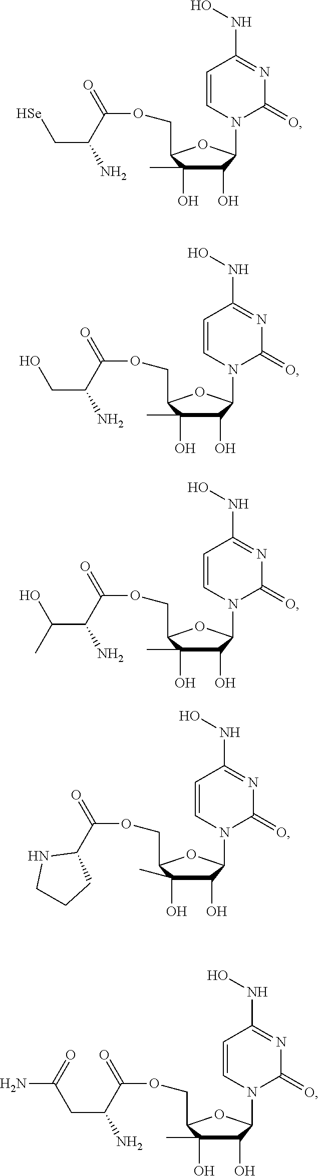

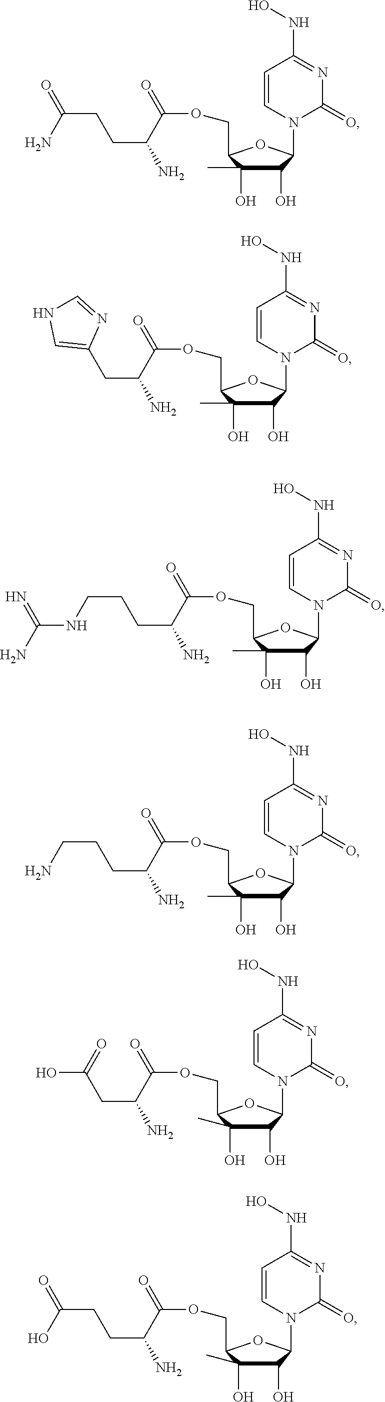

or a pharmaceutically-acceptable salt or prodrug thereof.

In another embodiment, any of the compounds of Formulas A-H can be present in the form of 5′-valine or isobutyl esters.

### III. Compound Preparation

In certain embodiments, the disclosure relates to method of making compounds disclosed herein by mixing starting materials and reagents disclosed herein under conditions such that the compounds are formed.

Methods for the facile preparation of active compounds are known in the art and result from the selective combination known methods. The compounds disclosed herein can be prepared as described in detail below, or by other methods known to those skilled in the art. It will be understood by one of ordinary skill in the art that variations of detail can be made without departing from the spirit and in no way limiting the scope of the present invention.

Some compounds within certain of the general formulas described herein are commercially available. For some compounds, the syntheses described herein are exemplary and can be used as a starting point to prepare additional compounds of the formulas described herein.

These compounds can be prepared in various ways, including those synthetic schemes shown and described herein. Those skilled in the art will be able to recognize modifications of the disclosed syntheses and to devise routes based on the disclosures herein; all such modifications and alternate routes are within the scope of the claims.

Certain terms used herein are commonly used and known to those skilled in the art. As used herein, the following abbreviations have the indicated meanings:
DMSO dimethylsulfoxide
DCM dichloromethane
DMAP 4-dimethylaminopyridine
EtOAc (AcOEt) ethyl acetate
HOAc Acetic acid
h hour
hex hexane
DIPEA Diisopropylethylamine
Liq. Liquid
LCMS Liquid chromatography mass spectrometry
TLC thin layer chromatography
M molar
MeOH Methanol
EtOH Ethanol
iPrOH Isopropyl alcohol
nBuOH n-Butyl alcohol
pTsOH p-Toluene sulfonic acid
TMSCN Trimethylsilylcyanide
TMSCl Trimethylsilylchloride
TMSOTf Trimethylsilyltriflate
Et₃N Triethylamine
nBuLi n-Butyl lithium
min minute
rt or RT room temperature
TBAF Tetrabutylammonium fluoride
THF tetrahydrofuran

The various reaction schemes are summarized below.

Scheme 1 A synthetic approach to nucleosides 4.

Scheme 2 A synthetic approach to nucleosides 6.

Scheme 3 A synthetic approach to nucleosides 9.

Scheme 4 An alternative synthetic approach to nucleosides 9.

Variables listed in the Schemes are as defined in active compound section.

Compounds of Formulas A and B can be prepared by first preparing nucleosides 4, which in turn can be accomplished by one of ordinary skill in the art, using methods outlined in: (a) Rajagopalan, P.; Boudinot, F. D; Chu, C. K.; Tennant, B. C.; Baldwin, B. H.; Antiviral Nucleosides: Chiral Synthesis and Chemotheraphy: Chu, C. K.; Eds. Elsevier: 2003. b) Recent Advances in Nucleosides: Chemistry and Chemotherapy: Chu, C. K.; Eds. Elsevier: 2002. c) Frontiers in Nucleosides & Nucleic Acids, 2004, Eds. R. F. Schinazi & D. C. Liotta, IHIL Press, Tucker, GA, USA, pp: 319-37 d) Handbook of Nucleoside Synthesis: Vorbruggen H. & Ruh-Pohlenz C. John Wiley & sons 2001), and by general Schemes 1-2. Specifically, nucleosides 2 can be prepared by coupling sugar 1 with a protected, silylated or free nucleoside base in the presence of Lewis acid such as TMSOTf. Deprotection of the 3′- and 5′-hydroxyls gives nucleoside 3. Selective protection of the 2′- and 5′-positions can be accomplished using well established sequences such as 1) reaction with 1,3-dichloro-1,1,3,3-tetraisopropyldisiloxan (TIPSCl₂) in presence of a base such as pyridine, 2) protection of the 3′-hydroxyl with a Trityl group with TrCl in presence of a base, 3) selective deprotection of the 3′ and 5′-position using in the case of a TIPS group, Bu₄NF salts and 4) selective protection of the 5′-position by reaction with for instance TrCl or TBDMSCl in presence of a base. Oxidation of the 2′-position with, for instance Dess Martin periodinane, followed by reduction with a deuterated reducing agent such as NaBD₄ and protection of the 2′-hydroxyl position with, for instance TBDMSCl in presence of a base, will give access to deuterated intermediate 4.

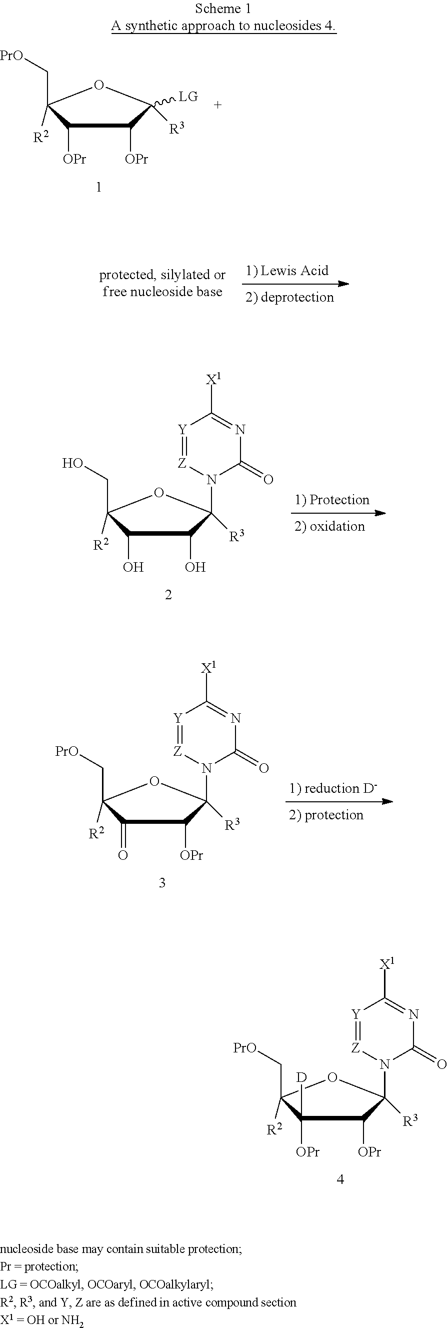

Deuterated nucleosides intermediates of general formula 6 can also be synthesized from deuterated intermediates by adapting the chemistry described in scheme 2 and in Ajmera et al., Labelled Compd. 1986, 23, 963; Sinhababu, et al., J. Am. Chem. Soc. 1985, 107, 7628; Robins, et al., Org. Chem. 1990, 55, 410; David, S. and Eustache, J., Carbohyd. Res. 1971, 16, 46 and David, S. and Eustache, J., Carbohyd. Res. 1971, 20.

3′-Methyl substituted nucleosides of general formula 6 can be prepared by adapting the chemistry described in Scheme 2 and Sahabuddin et al., Org. Biomol. Chem., 2006, 4, 551

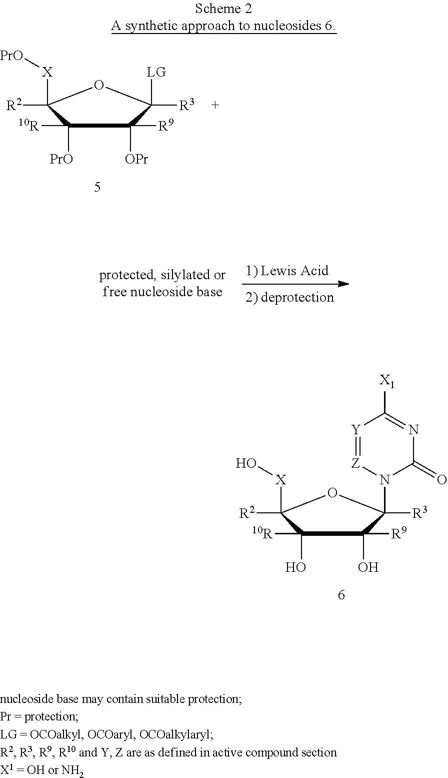

In the case of carbocyclic nucleosides, methods outlined above and in the following references can be adapted and used:
Advances in the enantioselective synthesis of carbocyclic nucleosides, Chem. Soc. Rev., 2013, 42, 5056
The latest progress in the synthesis of carbocyclic nucleosides”. Nucleosides, Nucleotides & Nucleic Acids. 2000, 19 (3): 651-690
New progresses in the enantioselective synthesis and biological properties of carbocyclic nucleosides”. Mini Reviews in Medicinal Chemistry 2003, 3(2): 95-114.
Chemical synthesis of carbocyclic analogues of nucleosides”. Chemical Synthesis of Nucleoside Analogues. Hoboken: John Wiley & Sons. 2003 pp. 535-604

Compounds of formula 9 can be prepared by adapting the chemistry described in Schemes 3 and 4. Compound 7 can be activated by, for instance reaction with POCl₃, or 2,4,6-triisopropylbenzenesulfonyl chloride in presence of base such as pyridine to give intermediate 8. Reaction of 8 with either HONH₂—HCl or BnONH₂—HCl in presence of a base such as, for instance DIPEA, followed by complete deprotecion of the corresponding compound gives access to nucleosides 9.

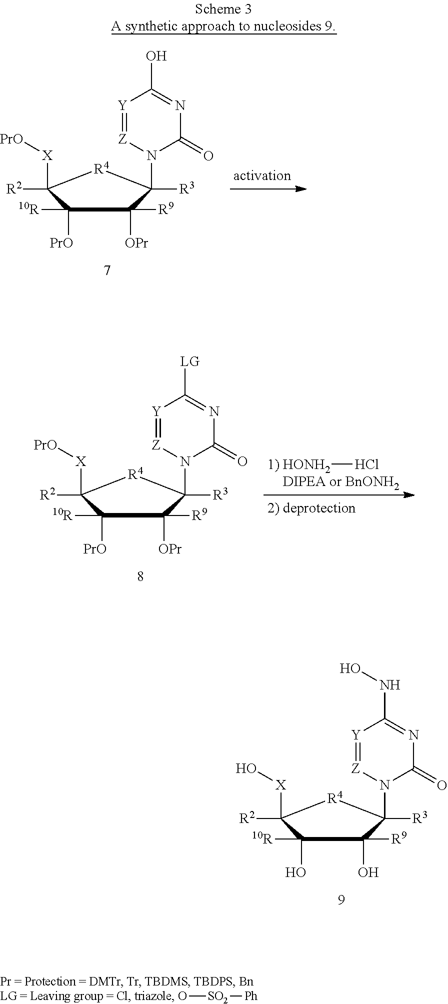

Alternatively, compound 9 can be prepared by reaction of nucloeside 10 with either HONH₂—HCl or BnONH₂—HCl followed by complete deprotection of the corresponding compound.

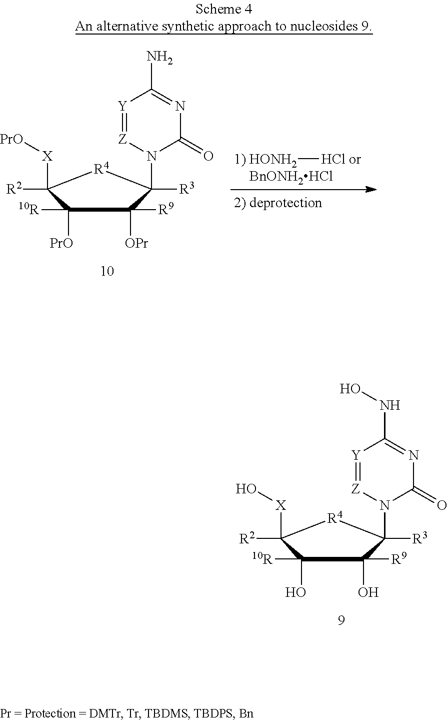

Compounds of Formulas A or B can also be prepared by adapting the chemistry described above and in: PCT WO 2019/113462.

Prodrugs of the desired compounds can be prepared by adapting the chemistry in: Pradere et al. Chem. Rev. 9154 (2014), or in PCT WO 2016/094677.

### Incorporation of Deuterium:

It is expected that single or multiple replacement of hydrogen with deuterium (carbon-hydrogen bonds to carbon-deuterium bond) at site(s) of metabolism in the sugar portion of a nucleoside antiviral agent will slow down the rate of metabolism. This can provide a relatively longer half-life, and slower clearance from the body. The slow metabolism of a therapeutic nucleoside is expected to add extra advantage to a therapeutic candidate, while other physical or biochemical properties are not affected. Intracellular hydrolysis or deuterium exchanges my result in liberation of deuterium oxide (D₂O).

Methods for incorporating deuterium into amino acids, phenol, sugars, and bases, are well known to those of skill in the art. Representative methods are disclosed in U.S. Pat. No. 9,045,521.

A large variety of enzymatic and chemical methods have been developed for deuterium incorporation at both the sugar and nucleoside stages to provide high levels of deuterium incorporation (D/H ratio). The enzymatic method of deuterium exchange generally has low levels of incorporation. Enzymatic incorporation has further complications due to cumbersome isolation techniques which are required for isolation of deuterated mononucleotide blocks. Schmidt et al., Ann. Chem. 1974, 1856; Schmidt et al., Chem. Ber., 1968, 101, 590, describes synthesis of 5′,5′-²H2-adenosine which was prepared from 2′,3′-O-isopropylideneadenosine-5′-carboxylic acid or from methyl-2,3-isopropylidene-beta-D-ribofuranosiduronic acid, Dupre, M. and Gaudemer, A., Tetrahedron Lett. 1978, 2783. Kintanar, et al., Am. Chem. Soc. 1998, 110, 6367 reported that diastereoisomeric mixtures of 5′-deuterioadenosine and 5′(R/S)-deuteratedthymidine can be obtained with reduction of the appropriate 5′-aldehydes using sodium borodeuteride or lithium aluminum deuteride (98 atom % ²H incorporation). Berger et al., Nucleoside & Nucleotides 1987, 6, 395 described the conversion of the 5′-aldehyde derivative of 2′deoxyguanosine to 5′ or 4′-deuterio-2′-deoxyguanosine by heating the aldehyde in ²H₂O/pyridine mixture (1:1) followed by reduction of the aldehyde with NaBD₄.

Ajmera et al., Labelled Compd. 1986, 23, 963 described procedures to obtain 4′-deuterium labeled uridine and thymidine (98 atom % ²H). Sinhababu, et al., J. Am. Chem. Soc. 1985, 107, 7628) demonstrated deuterium incorporation at the C3′ (97 atom % ²H) of adenosine during sugar synthesis upon stereoselective reduction of 1,2:5,6-di-O-isopropylidene-p-D-hexofuranos-3-ulose to 1,2:5,6-di-O-isopropylidene-3-deuterio-β-D-ribohexofuranose using sodium borodeuteride and subsequently proceeding further to the nucleoside synthesis. Robins, et al., Org. Chem. 1990, 55, 410 reported synthesis of more than 95% atom ²H incorporation at C3′ of adenosine with virtually complete stereoselectivity upon reduction of the 2′-O-tert-butyldimethylsilyl(TBDMS) 3-ketonucleoside by sodium borodeuteride in acetic acid. David, S. and Eustache, J., Carbohyd. Res. 1971, 16, 46 and David, S. and Eustache, J., Carbohyd. Res. 1971, 20, 319 described syntheses of 2′-deoxy-2′(S)-deuterio-uridine and cytidine. The synthesis was carried out by the use of 1-methyl-2-deoxy-2′-(S)-deuterio ribofuranoside.

Radatus, et al., J. Am. Chem. Soc. 1971, 93, 3086 described chemical procedures for synthesizing 2′-monodeuterated (R or S)-2′-deoxycytidines. These structures were synthesized from selective 2-monodeuterated-2-deoxy-D-riboses, which were obtained upon stereospecific reduction of a 2,3-dehydro-hexopyranose with lithium aluminum deuteride and oxidation of the resulting glycal. Wong et al. J. Am. Chem. Soc. 1978, 100, 3548 reported obtaining deoxy-1-deuterio-D-erythro-pentose, 2-deoxy-2(S)-deuterio-D-erythro-pentose and 2-deoxy-1,2(S)-dideuterio-D-erythro-pentose from D-arabinose by a reaction sequence involving the formation and LiAlD4 reduction of ketene dithioacetal derivatives.

Pathak et al. J., Tetrahedron 1986, 42, 5427) reported stereospecific synthesis of all eight 2′ or 2′-deuterio-2′-deoxynucleosides by reductive opening of appropriate methyl 2,3-anhydro-beta-D-ribo or beta-D-lyxofuranosides with LiAlD4. Wu et al. J. Tetrahedron 1987, 43, 2355 described the synthesis of all 2′,2″-dideuterio-2′-deoxynucleosides, for both deoxy and ribonucleosides, starting with oxidation of C2′ of sugar and subsequent reduction with NaBD₄ or LiAlD4 followed by deoxygenation by tributyltin deuteride. Roy et al. J. Am. Chem. Soc. 1986, 108, 1675, reported 2′,2′-dideuterio-2′-deoxyguanosine and thymidine can be prepared from 2-deoxyribose 5-phosphate using 2-deoxyribose 5-phosphate aldolase enzyme in ²H₂O achieving some 90 atom % deuteration. Similarly, the synthesis of 4′,5′,5′-²H₃-guanosine can be carried out.

Therefore, it is clear that each position of the sugar residue can be selectively labeled.

A useful alternative method of stereospecific deuteration was developed to synthesize polydeuterated sugars. This method employed exchange of hydrogen with deuterium at the hydroxyl bearing carbon (i.e. methylene and methine protons of hydroxyl bearing carbon) using deuterated Raney nickel catalyst in ²H₂O.

Various techniques are available to synthesize fully deuterated deoxy and ribonucleosides. Thus, in one method, exchange reaction of deuterated Raney nickel-²H₂O with sugars, a number of deuterated nucleosides specifically labeled at 2′, 3′ and 4′ positions were prepared. The procedure consisted of deuteration at 2′, 3′ and 4′ positions of methyl beta-D-arabinopyranoside by Raney nickel-²H₂O exchange reaction followed by reductive elimination of ′2-hydroxyl group by tributyltin deuteride to give methyl beta-D-2′,2′,3′,4′-²H₄-2-deoxyribopyranoside, which was converted to methyl beta-D-2′,2′,3′,4′-²H₄-2′-deoxyribofuranoside and glycosylated to give various 2′,2′,3′,4′-²H₄-nucleosides (>97 atom % ²H incorporation for H3′ & H4′.

The synthesis of deuterated phenols is described, for example, in Hoyer, H. (1950), Synthese des pan-Deutero-o-nitro-phenols. Chem. Ber., 83: 131-136. This chemistry can be adapted to prepare substituted phenols with deuterium labels. Deuterated phenols, and substituted analogs thereof, can be used, for example, to prepare phenoxy groups in phosphoramidate prodrugs.

The synthesis of deuterated amino acids is described, for example, in Matthews et al., Biochimica et Biophysica Acta (BBA)—General Subjects, Volume 497, Issue 1, 29 Mar. 1977, Pages 1-13. These and similar techniques can be used to prepare deuterated amino acids, which can be used to prepare phosphoramidate prodrugs of the nucleosides described herein.

One method for synthesizing a deuterated analog of the compounds described herein involves synthesizing a deuterated ribofuranoside with a 4′-alkynyl substitution; and attaching a nucleobase to the deuterated ribofuranoside to form a deuterated nucleoside. A prodrug, such as a phosphoramidate prodrug, can be formed by modifying the 5′—OH group on the nucleoside. Where a deuterated phenol and/or deuterated amino acid is used, one can prepare a deuterated phosphoramidate prodrug.

Another method involves synthesizing a ribofuranoside with 4′-alkynyl substitution, and attaching a deuterated nucleobase to form a deuterated nucleoside. This method can optionally be performed using a deuterated furanoside to provide additional deuteration. As with the method described above, the nucleoside can be converted into a prodrug form, which prodrug form can optionally include additional deuteration.

A third method involves synthesizing a ribofuranoside with 4′-alkynyl substitution, attaching a nucleobase to form a nucleoside, and converting the nucleoside to a phosphoramidate prodrug using one or both of a deuterated amino acid or phenol analog in the phosphoramidate synthesis.

A representative synthesis for preparing the deuterated nucleosides described herein can be adapted the chemistry described in Ajmera et al., Labelled Compd. 1986, 23, 963; Sinhababu, et al., J. Am. Chem. Soc. 1985, 107, 7628; Robins, et al., Org. Chem. 1990, 55, 410; David, S. and Eustache, J., Carbohyd. Res. 1971, 16, 46 and David, S. and Eustache, J., Carbohyd. Res. 1971, 20:

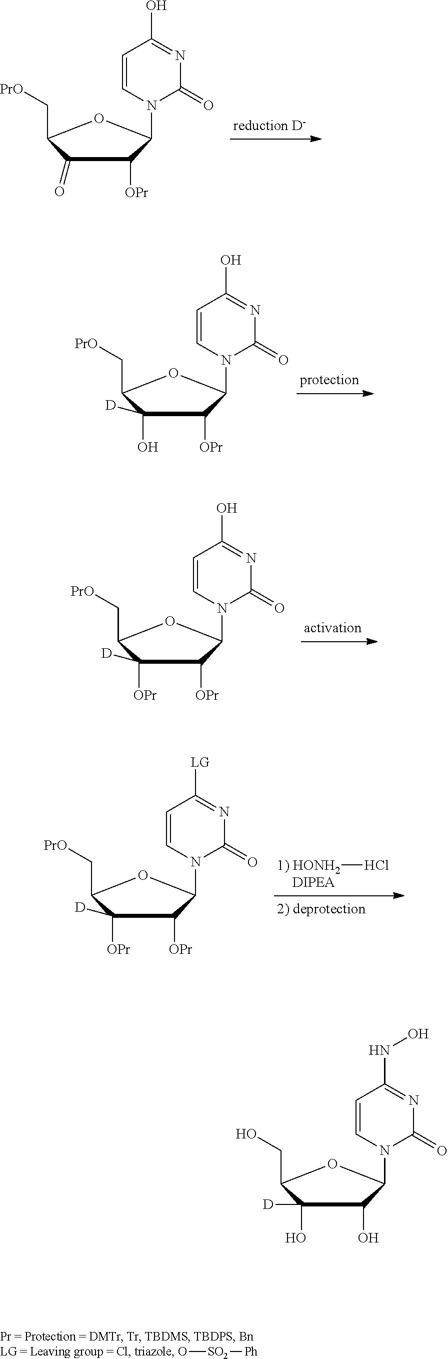

Accordingly, using the techniques described above, one can provide one or more deuterium atoms in the sugar, base, and/or prodrug portion of the nucleoside compounds described herein.

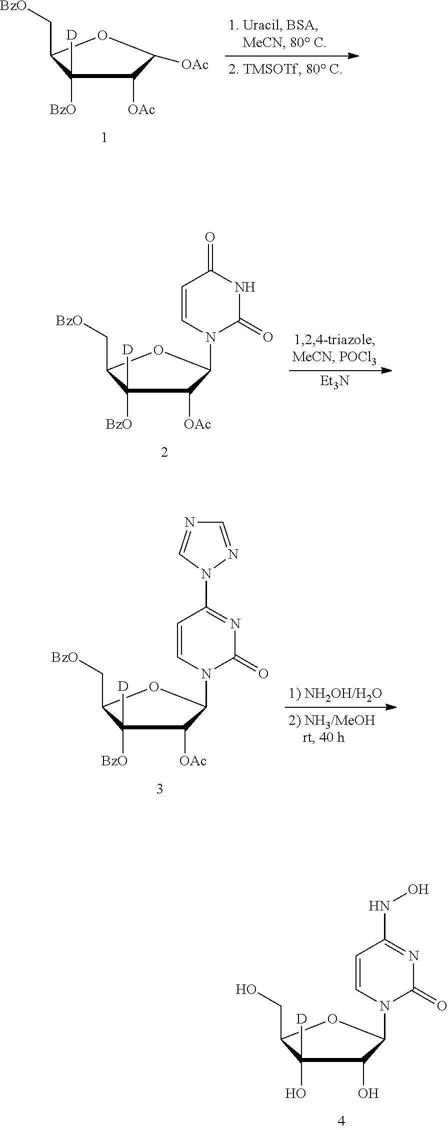

Synthesis of compound 1: Compound 1 is prepared by using the chemistry described in (a) A. K. Sinhababu, R. L. Bartel, N. Pochopin and R. T. Borchardt, Mechanism of Action of S-Adenosyl-L-homocysteine Hydrolase. Measurement of Kinetic Isotope Effects Using Adenosine-3′-d and S-Adenosyl-L-homocysteine-3′-d as Substrates J. Am. Chem. Soc. 1985, 107, 7628; (b) Y Saito, T A Zevaco and L A Agrofoglio, Chemical synthesis of ¹³C labeled anti-HIV nucleosides as mass-internal standards Tetrahedron, 2002, 58, 9593.

Synthesis of compound 2. To a mixture of uracil (1.13 g, 10.17 mmol) and 1(2.5 g, 5.6 mmol) in anhydrous acetonitrile (40 mL) was added BSA (4.7 mL, 19.25 mmol). The mixture was stirred at 80° C. for 30 min. After cooling down the reaction mixture to room temperature, TMSOTf (3.5 mL) was added dropwise. The reaction mixture was then stirred at 80° C. for 3 h for completion. The mixture was then diluted with EtOAc (200 mL), washed with sat. NaHCO₃(2×50 mL), brine (50 mL) and dried over Na₂SO₄. After removal of the volatiles under reduced pressure, the residue was purified by column chromatography (30 to 90% EtOAc/Hexane) to give compound 2 (2.35 g, 85%). ¹HNMR (CDCl₃): 9.81 (s, 1H), 8.07-8.05 (m, 4H), 7.63-7.44 (m, 6H), 7.37 (d, J=8.4 Hz, 1H), 6.21 (d, J=5.6 Hz, 1H), 5.60-5.56 (m, 2H), 4.80-4.61 (m, 3H), 2.05 (s, 3H); LCMS: m/z 496.3 (M+1)⁺.

Synthesis of compound 3: POCl₃ (90 μL, 0.95 mmol) was added dropwise to a stirred suspension of 1,2,4-triazole (310 mg, 4.5 mmol) in anhydrous acetonitrile (4 mL) at 0° C. After 10 min, triethylamine (0.6 mL, 4.3 mmol) was added dropwise at 0° C. After 50 min at 0° C., a solution of 2 (248 mg, 0.5 mmol) in anhydrous acetonitrile (5 mL) was added and the reaction was stirred at room temperature overnight. The reaction mixture was quenched with buffer (pH 7.4, 20 mL) and extracted with DCM (3×50 mL). The combined organic phases were dried over sodium sulfate. After removal of the volatiles under reduced pressure, the residue was purified by column chromatography (30 to 80% EtOAc/Hexane) to give compound 3 (190 mg). ¹H NMR (CDCl₃): 9.21 (s, 1H), 8.18-8.01 (m, 6H), 7.61-7.42 (m, 6H), 6.90 (d, J=7.2 Hz, 1H), 6.32 (d, J=4.8 Hz, 1H), 5.68 (d, J=4.8 Hz, 1H), 4.83-4.67 (m, 3H), 2.07 (s, 3H); LCMS: m/z 547.0 (M+1)⁺.

Synthesis of 3′-D-NHC 4: A solution of compound 3 in acetonitrile (10 mL) and 50% hydroxylamine (0.2 mL). was stirred for 15 min at room temperature. After removal of the volatils under reduced pressure, the residue was purified by column chromatography (0 to 15% MeOH/DCM). The intermediate was stirred at room temperature for 40 h in sat. NH₃/MeOH (100 mL). After removal of the volatiles under reduced pressure, the residue was purified by column chromatography (0 to 25% MeOH/DCM) to give product 4 (45 mg, 34%). ¹HNMR (CD₃OD): 7.16 (d, J=8.4 Hz, 1H), 5.87 (d, J=5.6 Hz, 1H), 5.62 (d, J=8.4 Hz, 1H), 4.16 (d, J=5.6 Hz, 1H), 3.95 (t, J=3.2 Hz, 1H), 3.78 (dd, J=12.0, 2.8 Hz, 1H), 3.70 (dd, J=12.0, 3.2 Hz, 1H); LCMS: m/z 261.3 (M+1)⁺.

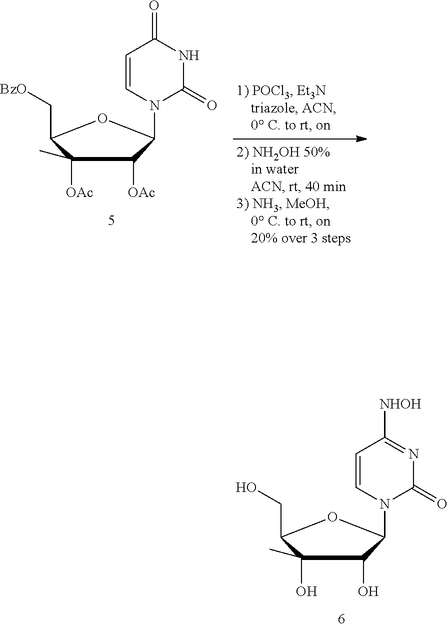

Synthesis of compound 5: Compound 5 is prepared by using the chemistry described in

H. Ko, R. L. Carter, L. Cosyn, R. Petrelli, S. de Castro, P. Besada, Y. Zhou, L. Cappellacci, P. Franchetti, M. Grifantini, S. Van Calenbergh, T. K. Harden, K. A. Jacobson. Synthesis and potency of novel uracil nucleotides and derivatives as P2Y2 and P2Y6 receptor agonists. Bioorg. Med. Chem. 2008, 16, 6319.

Synthesis of compound 6: To a solution 1,2,4-triazole (1.53 g, 22.2 mmol) in ACN (42 ml) was added Et₃N (3.3 mL, 23.9 mmol) and POCl₃ (0.35 mL, 3.7 mmol) at 0° C. The mixture was stirred at 0° C. for 3 hours, then 5 (0.32 g, 0.7 mmol) in ACN (9 ml) was added dropwise at 0° C. and the resulting solution was stirred overnight at room temperature. The reaction was diluted with EtOAc (50 mL), filtered off, washed with sat. NaHCO₃(10 ml) and brine (10 ml). The organic phase was concentrated under vacuum and purified by flash column chromatography (0 to 80% EtOAc/Hexane) to afford the desired triazole intermediate. To a solution of the triazole intermediate in ACN (3.1 mL) was added NH₂OH 50% in water (0.31 mL) at room temperature. The resulting solution was stirred for 40 min before evaporation of the volatiles. The residue was purified by flash chromatography (0 to 10% MeOH/DCM) to afford a mixture of partially deprotected intermediates. That mixture was dissolved in MeOH (16.4 mL) and NH_{3 (g)} was bubbled through that solution at 0° C. until saturation. The reaction was stirred at room temperature overnight, then evaporated under vacuum and purified by flash chromatography (0 to 15% MeOH/DCM) to give the title compound (67 mg, 20% over 3 steps) as a white foam. ¹H NMR (400 MHz, Methanol-d₄) δ 0.39 (d, 1H, J=8.3 Hz), 5.95 (d, 1H, J=7.9 Hz), 5.62 (d, 1H, J=9.4 Hz), 4.03 (d, 1H, J=8.0 Hz), 3.90 (t, 1H, J=2.8 Hz), 3.77-3.65 (m, 2H), 1.37 (s, 3H). HRMS-ESI (m z) [M+H]⁺ calcd.274.0961. for C₁₀H₁₆N₃O₅, found 274.1040.

### IV. Stereoisomerism and Polymorphism

The compounds described herein can have asymmetric centers and occur as racemates, racemic mixtures, individual diastereomers or enantiomers, with all isomeric forms being included in the present invention. Compounds of the present invention having a chiral center can exist in and be isolated in optically active and racemic forms. Some compounds can exhibit polymorphism. The present invention encompasses racemic, optically-active, polymorphic, or stereoisomeric forms, or mixtures thereof, of a compound of the invention, which possess the useful properties described herein. The optically active forms can be prepared by, for example, resolution of the racemic form by recrystallization techniques, by synthesis from optically-active starting materials, by chiral synthesis, or by chromatographic separation using a chiral stationary phase or by enzymatic resolution. One can either purify the respective compound, then derivatize the compound to form the compounds described herein, or purify the compound themselves.

Optically active forms of the compounds can be prepared using any method known in the art, including but not limited to by resolution of the racemic form by recrystallization techniques, by synthesis from optically-active starting materials, by chiral synthesis, or by chromatographic separation using a chiral stationary phase.

Examples of methods to obtain optically active materials include at least the following.
i) physical separation of crystals: a technique whereby macroscopic crystals of the individual enantiomers are manually separated. This technique can be used if crystals of the separate enantiomers exist, i.e., the material is a conglomerate, and the crystals are visually distinct;
ii) simultaneous crystallization: a technique whereby the individual enantiomers are separately crystallized from a solution of the racemate, possible only if the latter is a conglomerate in the solid state;
iii) enzymatic resolutions: a technique whereby partial or complete separation of a racemate by virtue of differing rates of reaction for the enantiomers with an enzyme;
iv) enzymatic asymmetric synthesis: a synthetic technique whereby at least one step of the synthesis uses an enzymatic reaction to obtain an enantiomerically pure or enriched synthetic precursor of the desired enantiomer;
v) chemical asymmetric synthesis: a synthetic technique whereby the desired enantiomer is synthesized from an achiral precursor under conditions that produce asymmetry (i.e., chirality) in the product, which can be achieved using chiral catalysts or chiral auxiliaries;
vi) diastereomer separations: a technique whereby a racemic compound is reacted with an enantiomerically pure reagent (the chiral auxiliary) that converts the individual enantiomers to diastereomers. The resulting diastereomers are then separated by chromatography or crystallization by virtue of their now more distinct structural differences and the chiral auxiliary later removed to obtain the desired enantiomer;
vii) first- and second-order asymmetric transformations: a technique whereby diastereomers from the racemate equilibrate to yield a preponderance in solution of the diastereomer from the desired enantiomer or where preferential crystallization of the diastereomer from the desired enantiomer perturbs the equilibrium such that eventually in principle all the material is converted to the crystalline diastereomer from the desired enantiomer. The desired enantiomer is then released from the diastereomer;
viii) kinetic resolutions: this technique refers to the achievement of partial or complete resolution of a racemate (or of a further resolution of a partially resolved compound) by virtue of unequal reaction rates of the enantiomers with a chiral, non-racemic reagent or catalyst under kinetic conditions;
ix) enantiospecific synthesis from non-racemic precursors: a synthetic technique whereby the desired enantiomer is obtained from non-chiral starting materials and where the stereochemical integrity is not or is only minimally compromised over the course of the synthesis;
x) chiral liquid chromatography: a technique whereby the enantiomers of a racemate are separated in a liquid mobile phase by virtue of their differing interactions with a stationary phase (including but not limited to via chiral PLC). The stationary phase can be made of chiral material or the mobile phase can contain an additional chiral material to provoke the differing interactions;
xi) chiral gas chromatography: a technique whereby the racemate is volatilized and enantiomers are separated by virtue of their differing interactions in the gaseous mobile phase with a column containing a fixed non-racemic chiral adsorbent phase;
xii) extraction with chiral solvents: a technique whereby the enantiomers are separated by virtue of preferential dissolution of one enantiomer into a particular chiral solvent;
xiii) transport across chiral membranes: a technique whereby a racemate is placed in contact with a thin membrane barrier. The barrier typically separates two miscible fluids, one containing the racemate, and a driving force such as concentration or pressure differential causes preferential transport across the membrane barrier. Separation occurs as a result of the non-racemic chiral nature of the membrane that allows only one enantiomer of the racemate to pass through.

Chiral chromatography, including but not limited to simulated moving bed chromatography, is used in one embodiment. A wide variety of chiral stationary phases are commercially available.

### V. Pharmaceutically-Acceptable Salts or Prodrugs

Representative pharmaceutically-acceptable salts and prodrugs are described below.

### Pharmaceutically-Acceptable Salts

In cases where compounds are sufficiently basic or acidic to form stable nontoxic acid or base salts, administration of the compound as a pharmaceutically acceptable salt may be appropriate. Accordingly, the term “pharmaceutically acceptable salts” includes acid addition and base salts thereof.

Examples of pharmaceutically acceptable salts are organic acid addition salts formed with acids which form non-toxic salts, i.e., which form a physiological-acceptable anion. and Suitable acid addition salts are formed from acids. Examples include the acetate, adipate, ascorbate, aspartate, benzoate, besylate, bicarbonate/carbonate, bisulphate/sulphate, borate, camsylate, citrate, cyclamate, edisylate, esylate, formate, fumarate, α-glycerophosphate, gluceptate, gluconate, glucuronate, hexafluorophosphate, hibenzate, hydrochloride/chloride, hydrobromide/bromide, hydroiodide/iodide, isethionate, α-ketoglutarate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts.

Suitable inorganic salts can also be formed, including but not limited to, sulfate, nitrate, bicarbonate and carbonate salts.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts.

Hemisalts of acids and bases can also be formed, for example, hemisulphate and hemicalcium salts. For a review on suitable salts, see Handbook of Pharmaceutical Salts: Properties, Selection, and Use by Stahl and Wermuth (Wiley-VCH, 2002), incorporated herein by reference.

For certain transdermal applications, it can be preferred to use fatty acid salts of the compounds described herein. The fatty acid salts can help penetrate the stratum corneum. Examples of suitable salts include salts of the compounds with stearic acid, oleic acid, lineoleic acid, palmitic acid, caprylic acid, and capric acid.

Physiologically acceptable salts of the exemplary compounds are those that are formed internally in a subject administered compound for the treatment or prevention of disease. Suitable salts include those of lithium, sodium, potassium, magnesium, calcium, manganese, bile salts.

Pharmaceutically acceptable salts can be obtained using standard procedures well known in the art, for example by reacting a sufficiently basic compound such as an amine with a suitable acid, affording a physiologically acceptable anion. In those cases where a compound includes multiple amine groups, the salts can be formed with any number of the amine groups. Alkali metal (e.g., sodium, potassium or lithium) or alkaline earth metal (e.g., calcium) salts of carboxylic acids can also be made.

### Prodrugs

Any of the nucleosides described herein can be administered as a nucleotide prodrug to increase the activity, bioavalability, stability or otherwise alter the properties of the nucleoside. A number of nucleotide prodrug ligands are known, including “protides,” which includes phosphoramidates.

A prodrug is a pharmacological substance that is administered in an inactive (or significantly less active) form and subsequently metabolized in vivo to an active metabolite. Getting more drug to the desired target at a lower dose is often the rationale behind the use of a prodrug and is generally attributed to better absorption, distribution, metabolism, and/or excretion (ADME) properties.

Prodrugs are usually designed to improve oral bioavailability, with poor absorption from the gastrointestinal tract usually being the limiting factor. Additionally, the use of a prodrug strategy can increase the selectivity of the drug for its intended target thus reducing the potential for off target effects.

A prodrug can include a covalently bonded carrier that releases the active parent drug when administered to a mammalian subject. Prodrugs can be prepared by modifying functional groups present in the compounds in such a way that the modifications are cleaved, either in routine manipulation or in vivo, to the parent compounds. Prodrugs include, for example, compounds wherein a hydroxyl group is bonded to any group that, when administered to a subject, cleaves to form a free hydroxyl group.

Examples of prodrugs that can be used to improve bioavailability include esters, such as isobutyl and valinyl esters, optionally substituted esters, branched esters, optionally substituted branched esters, carbonates, optionally substituted carbonates, carbamates, optionally substituted carbamates, thioesters, optionally substituted thioesters, branched thioesters, optionally substituted branched thioesters, thiocarbonates, optionally substituted thiocarbonates, S-thiocarbonate, optionally substituted S-thiocarbonate, dithiocarbonates, optionally substituted dithiocarbonates, thiocarbamates, optionally substituted thiocarbamates, oxymethoxy carbonyl, optionally substituted oxymethoxycarbonyl, oxymethoxythiocarbonyl, optionally substituted oxymethoxythiocarbonyl, oxymethylcarbonyl, optionally substituted oxymethylcarbonyl, oxymethylthiocarbonyl, optionally substituted oxymethylthiocarbonyl, L-amino acid esters, D-amino acid esters, N-substituted L-amino acid esters, N,N-disubstituted L-amino acid esters, N-substituted D-amino acid esters, N,N-disubstituted D-amino acid esters, sulfenyl, optionally substituted sulfenyl, imidate, optionally substituted imidate, hydrazonate, optionally substituted hydrazonate, oximyl, optionally substituted oximyl, imidinyl, optionally substituted imidinyl, imidyl, optionally substituted imidyl, aminal, optionally substituted aminal, hemiaminal, optionally susbstituted hemiaminal, acetal, optionally substituted acetal, hemiacetal, optionally susbstituted hemiacetal, carbonimidate, optionally substituted carbonimidate, thiocarbonimidate, optionally substituted thiocarbonimidate, carbonimidyl, optionally substituted carbonimidyl, carbamimidate, optionally substituted carbamimidate, carbamimidyl, optionally substituted carbamimidyl, thioacetal, optionally substituted thioacetal, S-acyl-2-thioethyl, optionally substituted S-acyl-2-thioethyl, bis-(acyloxybenzyl)esters, optionally substituted bis-(acyloxybenzyl)esters, (acyloxybenzyl)esters, optionally substituted (acyloxybenzyl)esters, and BAB-esters.

In one embodiment, the prodrug is an ester derivative, such as an acetate, formate and benzoate derivative, of one or more alcohol functional groups in the compounds.

Methods of structuring a compound as prodrugs can be found in the book of Testa and Mayer, Hydrolysis in Drug and Prodrug Metabolism, Wiley (2006). Typical prodrugs form the active metabolite by transformation of the prodrug by hydrolytic enzymes, the hydrolysis of amide, lactams, peptides, carboxylic acid esters, epoxides or the cleavage of esters of inorganic acids.

In general, alkylation, acylation or other lipophilic modification of the mono, di or triphosphoate of the nucleoside will increase the stability of the nucleotide. Examples of substituent groups that can replace one or more hydrogens on the phosphate moiety are alkyl, aryl, steroids, carbohydrates, including sugars, 1,2-diacylglycerol and alcohols. Many are described in R Jones and N. Bischofberger, Antiviral Research, 27 (1995) 1-17. Any of these can be used in combination with the disclosed nucleosides to achieve a desired effect. Nonlimiting examples of nucleotide prodrugs are described in the following references.

Ho, D. H. W. (1973) Distribution of Kinase and deaminase of 1.beta.-D-arabinofiuranosylcytosine in tissues of man and muse. Cancer Res. 33, 2816-2820; Holy, A. (1993) Isopolar phosphorous-modified nucleotide analogues. In: De Clercq (Ed.), Advances in Antiviral Drug Design, Vol. 1, JAI Press, pp. 179-23 1; Hong, C. I., Nechaev, A., and West, C. R. (1979a) Synthesis and antitumor activity of 1.beta.-D-arabinofuranosylcytosine conjugates of cortisol and cortisone. Biochem. Biophys. Rs. Commun. 88, 1223-1229; Hong, C. I., Nechaev, A., Kirisits, A. J. Buchheit, D. J. and West, C. R. (1980) Nucleoside conjugates as potential antitumor agents. 3. Synthesis and antitumor activity of 1-(.beta.-D-arabinofiuranosyl)cytosine conjugates of corticosteriods and selected lipophilic alcohols. J. Med. Chem. 28, 171-177; Hostetler, K. Y., Stuhmiller, L. M., Lenting, H. B. M. van den Bosch, H. and Richman, D. D. (1990) Synthesis and antiretrioviral activity of phospholipid analogs of azidothymidine and other antiviral nucleosides. J Biol. Chem. 265, 6112-6117; Hostetler, K. Y., Carson, D. A. and Richman, D. D. (1991); Phosphatidylazidothymidine: mechanism of antiretroviral action in CEM cells. J. Biol. Chem. 266, 11714-11717; Hostetler, K. Y., Korba, B. Sridhar, C., Gardener, M. (1994a) Antiviral activity of phosphatidyl-dideoxycytidine in hepatitis B-infected cells and enhanced hepatic uptake in mice. Antiviral Res. 24, 59-67; Hostetler, K. Y., Richman, D. D., Sridhar, C. N. Felgner, P. L, Felgner, J., Ricci, J., Gardener, M. F. Selleseth, D. W. and Ellis, M. N. (1994b) Phosphatidylazidothymidine and phosphatidyl-ddC: Assessment of uptake in mouse lymphoid tissues and antiviral activities in human immunodeficiency virus-infected cells and in rauscher leukemia virus-infected mice. Antimicrobial Agents Chemother. 38, 2792-2797; Hunston, R. N., Jones, A. A. McGuigan, C., Walker, R. T., Balzarini, J., and De Clercq, E. (1984) Synthesis and biological properties of some cyclic phosphotriesters derived from 2′-deoxy-5-fluorouridine. J. Med. Chem. 27, 440-444; Ji, Y. H., Moog, C., Schmitt, G., Bischoff, P. and Luu, B. (1990); Monophosphoric acid diesters of 7.beta.-hydroxycholesterol and of pytimidine nucleosides as potential antitumor agents: synthesis and preliminary evaluation of antitumor activity. J. Med Chem. 33, 2264-2270; Jones, A. S., McGuigan, C., Walker, R. T., Baizarini, J. and DeClercq, E. (1984) Synthesis, properties, and biological activity of some nucleoside cyclic phosphoramidates. J Chem. Soc. Perkin Trans. 1, 1471-1474; Juodka, B. A. and Smrt, J. (1974) Synthesis of ditribonucleoside phosph(P.fwdarw.N) amino acid derivatives. Coll. Czech. Chem. Comm. 39, 363-968; Kataoka, S., Imai, J., Yamaji, N., Kato, M., Saito, M., Kawada, T. and Imai, S. (1989) Alkylacted cAMP derivatives; selective synthesis and biological activities. Nucleic Acids Res. Sym. Ser., 21, 1-2; Kataoka, S., Uchida, R. and Yamaji, N. (1991) A convenient synthesis of adenosine 3′, 5′cyclic phosphate (cAMP) benzyl and methyl triesters. Heterocycles 32, 1351-1356; Kinchington, D., Harvey, J. J., O'Connor, T. J., Jones, B. C. N. M., Devine, K. G., Taylor-Robinson, D., Jeffries, D. J. and McGuigan, C. (1992) Comparison of antiviral effects of zidovudine phosphoramidate and phosphorodiamidate derivatives against HIV and ULV in vitro. Antiviral Chem. Chemother. 3, 107-112; Kodama, K., Morozumi, M., Saitoh, K. I., Kuninaka, H., Yoshino, H. and Saneyoshi, M. (1989) Antitumor activity and pharmacology of 1-.beta.-D-arabinofuranosylcytosine-5′-stearylphosphate; an orally active derivative of 1-.beta.-D-arabinofuranosylcytosine. Jpn. J. Cancer Res. 80, 679-685; Korty, M. and Engels, J. (1979) The effects of adenosine- and guanosine 3′, 5′phosphoric and acid benzyl esters on guinea-pig ventricular myocardium. Naunyn-Schmiedeberg's Arch. Pharmacol. 310, 103-111; Kumar, A., Goe, P. L., Jones, A. S. Walker, R. T. Balzarini, J. and De Clercq, E. (1990) Synthesis and biological evaluation of some cyclic phosphoramidate nucleoside derivatives. J. Med. Chem. 33, 2368-2375; LeBec, C., and Huynh-Dinh, T. (1991) Synthesis of lipophilic phosphate triester derivatives of 5-fluorouridine and arabinocytidine as anticancer prodrugs. Tetrahedron Lett. 32, 6553-6556; Lichtenstein, J., Bamer, H. D. and Cohen, S. S. (1960) The metabolism of exogenously supplied nucleotides by Escherichia coli., J. Biol. Chem. 235, 457-465; Lucthy, J., Von Daeniken, A., Friederich, J. Manthey, B., Zweifel, J., Schlatter, C. and Benn, M. H. (1981) Synthesis and toxicological properties of three naturally occurring cyanoepithioalkanes. Mitt. Geg. Lebensmittelunters. Hyg. 72, 131-133 (Chem. Abstr. 95, 127093); McGuigan, C. Tollerfield, S. M. and Riley, P. A. (1989) Synthesis and biological evaluation of some phosphate triester derivatives of the anti-viral drug Ara. Nucleic Acids Res. 17, 6065-6075; McGuigan, C., Devine, K. G., O'Connor, T. J., Galpin, S. A., Jeffties, D. J. and Kinchington, D. (1990a) Synthesis and evaluation of some novel phosphoramidate derivatives of 3′-azido-3′deoxythymidine (AZT) as anti-HIV compounds. Antiviral Chem. Chemother. 1, 107-113; McGuigan, C., O'Connor, T. J., Nicholls, S. R. Nickson, C. and Kinchington, D. (1990b) Synthesis and anti-HIV activity of some novel substituted dialky phosphate derivatives of AZT and ddCyd. Antiviral Chem. Chemother. 1, 355-360; McGuigan, C., Nicholls, S. R., O'Connor, T. J., and Kinchington, D. (1990c) Synthesis of some novel dialkyl phosphate derivative of 3′-modified nucleosides as potential anti-AIDS drugs. Antiviral Chem. Chemother. 1, 25-33; McGuigan, C., Devine, K. G., O'Connor, T. J., and Kinchington, D.(1991) Synthesis and anti-HIV activity of some haloalky phosphoramidate derivatives of 3′-azido-3′deoxythylmidine (AZT); potent activity of the trichloroethyl methoxyalaninyl compound. Antiviral Res. 15, 255-263; McGuigan, C., Pathirana, R. N., Malimood, N., Devine, K. G. and Hay, A. J. (1992) Aryl phosphate derivatives of AZT retain activity against HIV1 in cell lines which are resistant to the action of AZT. Antiviral Res. 17, 311-321; McGuigan, C., Pathirana, R. N., Choi, S. M., Kinchington, D. and O'Connor, T. J. (1993a) Phosphoramidate derivatives of AZT as inhibitors of HIV; studies on the carboxyl terminus. Antiviral Chem. Chemother. 4, 97-101; McGuigan, C., Pathirana, R. N., Balzarini, J. and De Clercq, E. (1993b) Intracellular delivery of bioactive AZT nucleotides by aryl phosphate derivatives of AZT. J. Med. Chem. 36, 1048-1052.

Alky hydrogen phosphonate derivatives of the compounds described herein may be less toxic than the parent nucleoside analogues. Antiviral Chem. Chemother. 5, 271-277; Meyer, R. B., Jr., Shuman, D. A. and Robins, R. K. (1973) Synthesis of purine nucleoside 3′, 5′-cyclic phosphoramidates. Tetrahedron Lett. 269-272; Nagyvary, J. Gohil, R. N., Kirchner, C. R. and Stevens, J. D. (1973) Studies on neutral esters of cyclic AMP, Biochem. Biophys. Res. Commun. 55, 1072-1077; Namane, A. Gouyette, C., Fillion, M. P., Fillion, G. and Huynh-Dinh, T. (1992) Improved brain delivery of AZT using a glycosyl phosphotriester prodrug. J. Med. Chem. 35, 3039-3044; Nargeot, J. Nerbonne, J. M. Engels, J. and Leser, H. A. (1983) Natl. Acad. Sci. U.S.A. 80, 2395-2399; Nelson, K. A., Bentrude, W. G., Stser, W. N. and Hutchinson, J. P. (1987) The question of chair-twist equilibria for the phosphate rings of nucleoside cyclic 3′, 5′monophosphates. HNMR and x-ray crystallographic study of the diasteromers of thymidine phenyl cyclic 3′, 5′-monophosphate. J. Am. Chem. Soc. 109, 4058-4064; Nerbonne, J. M., Richard, S., Nargeot, J. and Lester, H. A. (1984)New photoactivatable cyclic nucleotides produce intracellular jumps in cyclic AMP and cyclic GMP concentrations. Nature 301, 74-76; Neumann, J. M., Herve, M., Debouzy, J. C., Guerra, F. I., Gouyette, C., Dupraz, B. and Huynh-Dinh, T. (1989) Synthesis and transmembrane transport studies by NMR of a glucosyl phospholipid of thymidine. J. Am. Chem. Soc. 111, 42704277; Ohno, R., Tatsumi, N., Hirano, M., Imai, K. Mizoguchi, H., Nakamura, T., Kosaka, M., Takatuski, K., Yamaya, T., Toyama, K., Yoshida, T., Masaoka, T., Hashimoto, S., Ohshima, T., Kimura, I., Yamada, K. and Kimura, J. (1991) Treatment of myelodysplastic syndromes with orally administered 1-.beta.-D-rabinofuranosylcytosine-5′stearylphosphate. Oncology 48, 451-455; Palomino, E., Kessle, D. and Horwitz, J. P. (1989) A dihydropyridine carrier system for sustained delivery of 2′, 3′dideoxynucleosides to the brain. J. Med. Chem. 32, 622-625; Perkins, R. M., Barney, S., Wittrock, R., Clark, P. H., Levin, R. Lambert, D. M., Petteway, S. R., Serafinowska, H, T., Bailey, S. M., Jackson, S., Harnden, M. R. Ashton, R., Sutton, D., Harvey, J. J. and Brown, A. G. (1993) Activity of BRL47923 and its oral prodrug, SB203657A against a rauscher murine leukemia virus infection in mice. Antiviral Res. 20 (Suppl. I). 84; Piantadosi, C., Marasco, C. J., Jr., Morris-Natschke, S. L., Meyer, K. L., Gumus, F., Surles, J. R., Ishaq, K. S., Kucera, L. S. Iyer, N., Wallen, C. A., Piantadosi, S. and Modest, E. J. (1991) Synthesis and evaluation of novel ether lipid nucleoside conjugates for anti-HIV-1 activity. J. Med. Chem. 34, 1408-1414; Pompon, A., Lefebvre, I., Imbach, J. L., Kahn, S. and Farquhar, D. (1994) Decomposition pathways of the mono- and bis(pivaloyloxymethyl) esters of azidothymidine-5′-monophosphate in cell extract and in tissue culture medium; an application of the on-line ISRP-cleaning' IIPLC technique. Antiviral Chem. Chemother. 5, 91-98; Postemark, T. (1974) Cyclic AMP and cyclic GMP. Annu. Rev. Pharmacol. 14, 23-33; Prisbe, E. J., Martin, J. C. M., McGee, D. P. C., Barker, M. F., Smee, D. F. Duke, A. E., Matthews, T. R. and Verheyden, J. P. J. (1986) Synthesis and antiherpes virus activity of phosphate an phosphonate derivatives of 9-[(1,3-dihydroxy-2-propoxy)methyl]guanine. J. Med. Chem. 29, 671-675; Pucch, F., Gosselin, G., Lefebvre, I., Pompon, A., Aubertin, A. M. Dirn, A. and Imbach, J. L. (1993) Intracellular delivery of nucleoside monophosphate tbrough a reductase-mediated activation process. Antiviral Res. 22, 155-174; Pugaeva, V. P., Klochkeva, S. I., Mashbits, F. D. and Eizengart, R. S. (1969). Toxicological assessment and health standard ratings for ethylene sulfide in the industrial atmosphere. Gig. Trf. Prof. Zabol. 13, 47-48 (Chem. Abstr. 72, 212); Robins, R. K. (1984) The potential of nucleotide analogs as inhibitors of retroviruses and tumors. Pharm. Res. 11-18; Rosowsky, A., Kim. S. H., Ross and J. Wick, M. M. (1982) Lipophilic 5′-(alkylphosphate) esters of 1.beta.-D-arabinofuranosylcytosing and its N.sup.4-acyl and 2.2′-anhydro-3′-O-acyl derivatives as potential prodrugs. J. Med. Chem. 25, 171-178; Ross, W. (1961) Increased sensitivity of the walker turnout towards aromatic nitrogen mustards carrying basic side chains following glucose pretreatment. Biochem. Phaim. 8, 235-240; Ryu, e. K., Ross, R. J. Matsushita, T., MacCoss, M., Hong, C. I. and West, C. R. (1982). Phospholipid-nucleoside conjugates. 3. Synthesis and preliminary biological evaluation of 1-.beta.-D-arabinofuranosylcytosine 5′diphosphate[-], 2-diacylglycerols. J. Med. Chem. 25, 1322-1329; Saffliill, R. and Hume, W. J. (1986) The degradation of 5-iododeoxyurindine and 5-bromoeoxyuridine by serum from different sources and its consequences for the use of these compounds for incorporation into DNA. Chem. Biol. Interact. 57, 347-355; Saneyoshi, M., Morozumi, M., Kodama, K., Machida, J., Kuninaka, A. and Yoshino, H. (1980) Synthetic nucleosides and nucleotides. XVI. Synthesis and biological evaluations of a series of 1-.beta.-D-arabinofuranosylcytosine 5′-alky or arylphosphates. Chem. Pharm. Bull. 28, 2915-2923; Sastry, J. K., Nehete, P. N., Khan, S., Nowak, B. J., Plunkett, W., Arlinghaus, R. B. and Farquhar, D. (1992) Membrane-permeable dideoxyuridine 5′-monophosphate analogue inhibits human immunodeficiency virus infection. Mol. Pharimacol. 41, 441 445; Shaw, J. P., Jones, R. J. Arimilli, M. N., Louie, M. S., Lee, W. A. and Cundy, K. C. (1994) Oral bioavailability of PMEA from PMEA prodrugs in male Sprague-Dawley rats. 9th Annual AAPS Meeting. San Diego, Calif. (Abstract). Shuto, S., Ueda, S., Imamura, S., Fukukawa, K. Matsuda, A. and Ueda, T. (1987) A facile one-step synthesis of Sphosphatidylnucleosides by an enzymatic two-phase reaction. Tetrahedron Lett. 28, 199-202; Shuto, S., Itoh, H., Ueda, S., Imamura, S., Kukukawa, K., Tsujino, M., Matsuda, A. and Ueda, T. (1988) A facile enzymatic synthesis of 5′-(3-sn-phosphatidyl)nucleosides and their antileukemic activities. Chem. Pharm. Bull. 36, 209-217.

Examplary preferred phosphate prodrug groups include phosphoramidates, the S-acyl-2-thioethyl group, also referred to as “SATE”, and Carbonyloxymethyl group (Including POM, POC), Alkoxyalkyl Monoester group (HDP, ODE), Cyclosaligenyl (cycloSal) Phosphate, cyclic 1-Aryl-1,3-propanyl Ester HepDirect, and Bis(amino acid) 0-phosphorodiamidates.

Examplary prodrug forms are described in the various formulas, including in the definitions of variables R¹, R⁶, R⁷, R¹¹, and R¹².

Accordingly, those of skill in the art will readily understand which prodrug forms, including 5′-phosphate prodrugs, can be prepared for the nucleosides described herein.

### VI. Methods of Treatment

In certain embodiments, the disclosure relates to methods of treating or preventing a viral infection, comprising administering an effective amount of a compound or pharmaceutical composition disclosed herein to a subject in need thereof.

In certain embodiments, the compound is administered via pulmonary administration, such as by inhalation or nebulization. In other embodiments, the compound is administered orally, topically, intraveneously, intraarticularly, intramuscularly, subcutaneously, buccally, or transdermally.

In certain embodiments, methods for treating or preventing infections caused by RNA viruses, including, but not limited to, Coronaviridae, such as MERSr-CoV, SARS-CoV-1, SARS-CoV-2, HCoV-OC43, HCoV-229E, HCoV-NL63, and HKU1 (HCoV-HKU1, Picornaviridae, Hepeviridae, Noroviruses, Zika, Dengue, Mayaro, Influenza A and B, Parainfluenza, HCV, Rinovirus, tick-borne viruses, Ebola, Lassa, RSV, adenoviruses, enteroviruses, metapneumoviruses, Eastern, Western, and Venezuelan Equine Encephalitis (EEE, WEE and VEE, respectively), and Chikungunya fever (CHIK), are also disclosed.

In aspect of these embodiments, the Coronavirus is SARS, MERS, SARS-CoV-2 or HCoV-OC43.

In aspects of these embodiments, the viral infection is, or is caused by, an alphavirus, flavivirus, coronavirus, picornavirus, orthomyxoviridae or paramyxoviridae, RSV, influenza, Powassan virus, filoviridae or Ebola.

In certain embodiments, the viral infection is, or is caused by, a virus selected from Eastern equine encephalitis virus, Western equine encephalitis virus, Venezuelan equine encephalitis virus, Ross River virus, Barmah Forest virus, Powassan virus, Zika virus, and Chikungunya virus.

In another embodiment, a method of treating or preventing a Zika virus infection is provided, the method comprising administering an effective amount of a compound or pharmaceutical composition disclosed herein to a subject in need thereof.

In some embodiments, the subject is at risk of, exhibiting symptoms of, or diagnosed with an infection by an RNA virus, such as an influenza A virus, such as subtype H1N1, H3N2, H7N9, or H5N1, influenza B virus, influenza C virus, rotavirus A, rotavirus B, rotavirus C, rotavirus D, rotavirus E, human coronavirus, such as MERSr-CoV, SARS-CoV-1, SARS-CoV-2, HCoV-OC43, HCoV-229E, HCoV-NL63, and HCoV-HKU1, human adenovirus types (HAdV-1 to 55), human papillomavirus (HPV) Types 16, 18, 31, 33, 35, 39, 45, 51, 52, 56, 58, and 59, parvovirus B19, molluscum contagiosum virus, JC virus (JCV), BK virus, Merkel cell polyomavirus, coxsackie A virus, norovirus, Rubella virus, lymphocytic choriomeningitis virus (LCMV), Dengue virus, Zika virus, chikungunya, Eastern equine encephalitis virus (EEEV), Western equine encephalitis virus (WEEV), Venezuelan equine encephalitis virus (VEEV), Ross River virus, Barmah Forest virus, yellow fever virus, measles virus, mumps virus, respiratory syncytial virus, rinderpest virus, California encephalitis virus, hantavirus, rabies virus, ebola virus, marburg virus, varicella zoster virus (VZV), Epstein-Barr virus (EBV), cytomegalovirus (CMV), roseolovirus, hepatitis A, hepatitis C, hepatitis D, hepatitis E or human immunodeficiency virus (HIV), the Human T-lymphotropic virus Type I (HTLV-1), Friend spleen focus-forming virus (SFFV), Xenotropic MuLV-Related Virus (XMRV), or a Zika virus.

In certain embodiments, the subject is diagnosed with influenza A virus including subtypes H1N1, H3N2, H7N9, H5N1 (low path), and H5N1 (high path) influenza B virus, influenza C virus, rotavirus A, rotavirus B, rotavirus C, rotavirus D, rotavirus E, SARS coronavirus, such as MERSr-CoV, SARS-CoV-1, SARS-CoV-2, HCoV-OC43, HCoV-229E, HCoV-NL63, and HCoV-HKU1, human adenovirus types (HAdV-1 to 55), human papillomavirus (HPV) Types 16, 18, 31, 33, 35, 39, 45, 51, 52, 56, 58, and 59, parvovirus B19, molluscum contagiosum virus, JC virus (JCV), BK virus, Merkel cell polyomavirus, coxsackie A virus, norovirus, Rubella virus, lymphocytic choriomeningitis virus (LCMV), yellow fever virus, measles virus, mumps virus, respiratory syncytial virus, parainfluenza viruses 1 and 3, rinderpest virus, chikungunya, eastern equine encephalitis virus (EEEV), Venezuelan equine encephalitis virus (VEEV), western equine encephalitis virus (WEEV), California encephalitis virus, Japanese encephalitis virus, Rift Valley fever virus (RVFV), hantavirus, Dengue virus serotypes 1, 2, 3 and 4, Zika virus, West Nile virus, Tacaribe virus, Junin, rabies virus, ebola virus, marburg virus, adenovirus, varicella zoster virus (VZV), Epstein-Barr virus (EBV), cytomegalovirus (CMV), roseolovirus, hepatitis A, hepatitis C, hepatitis D, hepatitis E, human immunodeficiency virus (HIV), or a Zika virus.

In certain embodiments, the subject is diagnosed with gastroenteritis, acute respiratory disease, severe acute respiratory syndrome, post-viral fatigue syndrome, viral hemorrhagic fevers, acquired immunodeficiency syndrome or hepatitis.

### Treatment of Pulmonary Infections

The methods described herein can be used to treat, prevent, manage or lessen the severity of symptoms and infections associated with one or more pulmonary diseases or infections in a subject. The methods involve administering one or more of the compounds described herein to the subject. The compounds can be administered to the mouth, nasal passages, throat, esophagus, larynx, pharynx, trachea, bronchioles, bronchi, upper airways, lower airways, subcutaneously or via an implant (for example, up under the ribs and into the chest cavity), and combinations thereof.

In some embodiments, the compounds are nebulized, inhaled, or delivered intranasally. In one specific embodiment, the methods comprise inhalation of particles including one or more of the compounds described herein aerosolized via nebulization. Nebulizers generally use compressed air or ultrasonic power to create inhalable aerosol droplets of the particles or suspensions thereof. In this embodiment, the nebulizing results in pulmonary delivery to the subject of aerosol droplets of the particles or suspension thereof. In another embodiment, the methods comprise inhalation of particles aerosolized via a pMDI, wherein the particles or suspensions thereof are suspended in a suitable propellant system (including but not limited to hydrofluoroalkanes (HFAs) containing at least one liquefied gas in a pressurized container sealed with a metering valve. Actuation of the valve results in delivery of a metered dose of an aerosol spray of the particles or suspensions thereof.

In one embodiment, the compounds are administered during lung lavage, which can be whole lung lavage or bronchoalveolar lavage (BAL). In BAL, also known as bronchoalveolar washing, a bronchoscope is passed through the mouth or nose into the lungs and fluid is squirted into a small part of the lung and then collected for examination. The compounds can travel through the fluid, and treat the entire fluid-coated portion of the lung.

Bronchoalveolar lavage is commonly used to diagnose infections in people with immune system problems, pneumonia in people on ventilators, some types of lung cancer, and scarring of the lung (interstitial lung disease). It is the most common method used to sample the epithelial lining fluid (ELF) and to determine the protein composition of the pulmonary airways. It is often used in immunological research as a means of sampling cells (for example, T cells) or pathogen levels (for example, influenza virus) in the lung. During the procedure, the compounds described herein can be administered. Whole lung lavage (WLL; or “lung washing”) is a treatment for pulmonary alveolar proteinosis. While the lung is washed, therapy with the compounds can also be administered, and the fluid allows the compounds to contact the entire fluid-coated surface of the lung.

In some embodiments, the compounds are used to treat or prevent microbial infections, including those caused by viruses such as Coronavirus, including MERSr-CoV, SARS-CoV-1, SARS-CoV-2, HCoV-OC43, HCoV-229E, HCoV-NL63, and HCoV-HKU1.

### VI. Pharmaceutical Compositions

Hosts, including but not limited to humans, infected with a Coronviridae virus, or the other viruses described, herein can be treated by administering to the patient an effective amount of the active compound or a pharmaceutically acceptable prodrug or salt thereof in the presence of a pharmaceutically acceptable carrier or diluent. The active materials can be administered by any appropriate route, for example, orally, parenterally, intravenously, intradermally, subcutaneously, or topically, in liquid or solid form.

A preferred dose of the compound for will be in the range of between about 0.01 and about 10 mg/kg, more generally, between about 0.1 and 5 mg/kg, and, preferably, between about 0.5 and about 2 mg/kg, of body weight of the recipient per day, until the patient has recovered. In some cases, a compound may be administered at a dosage of up to 10 μM, which might be considered a relatively high dose if administered for an extended period of time, but which can be acceptable when administered for the duration of an infection with one or more of the viruses described herein, which is typically on the order of several days to several weeks.

The effective dosage range of the pharmaceutically acceptable salts and prodrugs can be calculated based on the weight of the parent compound to be delivered. If the salt or prodrug exhibits activity in itself, the effective dosage can be estimated as above using the weight of the salt or prodrug, or by other means known to those skilled in the art.

The compound is conveniently administered in unit any suitable dosage form, including but not limited to but not limited to one containing 7 to 600 mg, preferably 70 to 600 mg of active ingredient per unit dosage form. An oral dosage of 5-400 mg is usually convenient.

The concentration of active compound in the drug composition will depend on absorption, inactivation and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The active ingredient can be administered at once, or can be divided into a number of smaller doses to be administered at varying intervals of time.

A preferred mode of administration of the active compound is oral. Oral compositions will generally include an inert diluent or an edible carrier. They can be enclosed in gelatin capsules or compressed into tablets. For the purpose of oral therapeutic administration, the active compound can be incorporated with excipients and used in the form of tablets, troches or capsules. Pharmaceutically compatible binding agents, and/or adjuvant materials can be included as part of the composition.

The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as a fatty oil. In addition, unit dosage forms can contain various other materials that modify the physical form of the dosage unit, for example, coatings of sugar, shellac, or other enteric agents.

The compound can be administered as a component of an elixir, suspension, syrup, wafer, chewing gum or the like. A syrup can contain, in addition to the active compound(s), sucrose as a sweetening agent and certain preservatives, dyes and colorings and flavors.

The compound or a pharmaceutically acceptable prodrug or salts thereof can also be mixed with other active materials that do not impair the desired action, or with materials that supplement the desired action, such as antibiotics, antifungals, anti-inflammatories or other antiviral compounds. Solutions or suspensions used for parenteral, intradermal, subcutaneous, or topical application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents, such as ethylenediaminetetraacetic acid; buffers, such as acetates, citrates or phosphates, and agents for the adjustment of tonicity, such as sodium chloride or dextrose. The parental preparation can be enclosed in ampoules, disposable syringes or multiple dose vials made of glass or plastic.

If administered intravenously, preferred carriers are physiological saline or phosphate buffered saline (PBS).

### Transdermal Formulations

In some embodiments, the compositions are present in the form of transdermal formulations, such as that used in the FDA-approved agonist rotigitine transdermal (Neupro patch). Another suitable formulation is that described in U.S. Publication No. 20080050424, entitled “Transdermal Therapeutic System for Treating Parkinsonism.” This formulation includes a silicone or acrylate-based adhesive, and can include an additive having increased solubility for the active substance, in an amount effective to increase dissolving capacity of the matrix for the active substance.

The transdermal formulations can be single-phase matrices that include a backing layer, an active substance-containing self-adhesive matrix, and a protective film to be removed prior to use. More complicated embodiments contain multiple-layer matrices that may also contain non-adhesive layers and control membranes. If a polyacrylate adhesive is used, it can be crosslinked with multivalent metal ions such as zinc, calcium, aluminum, or titanium ions, such as aluminum acetylacetonate and titanium acetylacetonate.

When silicone adhesives are used, they are typically polydimethylsiloxanes. However, other organic residues such as, for example, ethyl groups or phenyl groups may in principle be present instead of the methyl groups. Because the active compounds are amines, it may be advantageous to use amine-resistant adhesives. Representative amine-resistant adhesives are described, for example, in EP 0 180 377.

Representative acrylate-based polymer adhesives include acrylic acid, acrylamide, hexylacrylate, 2-ethylhexylacrylate, hydroxyethylacrylate, octylacrylate, butylacrylate, methylacrylate, glycidylacrylate, methacrylic acid, methacrylamide, hexylmethacrylate, 2-ethylhexylmethacrylate, octylmethacrylate, methylmethacrylate, glycidylmethacrylate, vinylacetate, vinylpyrrolidone, and combinations thereof.

The adhesive must have a suitable dissolving capacity for the active substance, and the active substance most be able to move within the matrix, and be able to cross through the contact surface to the skin. Those of skill in the art can readily formulate a transdermal formulation with appropriate transdermal transport of the active substance.

Certain pharmaceutically acceptable salts tend to be more preferred for use in transdermal formulations, because they can help the active substance pass the barrier of the stratum corneum. Examples include fatty acid salts, such as stearic acid and oleic acid salts. Oleate and stearate salts are relatively lipophilic, and can even act as a permeation enhancer in the skin.

Permeation enhancers can also be used. Representative permeation enhancers include fatty alcohols, fatty acids, fatty acid esters, fatty acid amides, glycerol or its fatty acid esters, N-methylpyrrolidone, terpenes such as limonene, alpha-pinene, alpha-terpineol, carvone, carveol, limonene oxide, pinene oxide, and 1,8-eucalyptol.

The patches can generally be prepared by dissolving or suspending the active agent in ethanol or in another suitable organic solvent, then adding the adhesive solution with stirring. Additional auxiliary substances can be added either to the adhesive solution, the active substance solution or to the active substance-containing adhesive solution. The solution can then be coated onto a suitable sheet, the solvents removed, a backing layer laminated onto the matrix layer, and patches punched out of the total laminate.

### Intranasal/Nebulizable Formulations

In some embodiments, the compounds are administered to the pulmonary tract (i.e., via pulmonary administration), for example, via intranasal administration or nebulization.

In one specific embodiment, pulmonary administration comprises inhalation of the compounds, typically in the form of particles or droplets, such as by nasal, oral inhalation, or both. The formulations can be developed to be aerosolized via a metered dose inhaler, a dry powder inhaler, a liquid spray or a nebulizer devises. Nebulization can be accomplished by compressed air, ultrasonic energy, or vibrating mesh to form a plurality of liquid droplets or solid particles comprising the NO-releasing compounds.

In one aspect of this embodiment, particles may be formulated as an aerosol (i.e., liquid droplets of a stable dispersion or suspension of particles that include one or more of the compounds described herein in a gaseous medium). Particles delivered by aerosol may be deposited in the airways by gravitational sedimentation, inertial impaction, and/or diffusion.

Whether administered by inhalation or nebulization, the particles or droplets can be administered in two or more separate administrations (doses).

Biodegradable particles can be used for the controlled-release and delivery of the compounds described herein. Aerosols for the delivery of therapeutic agents to the respiratory tract have been developed. Adjei, A. and Garren, J. Pharm Res. 7, 565-569 (1990); and Zanen, P. and Lamm, J.-W. J. Int. J. Pharm. 114, 111-115 (1995).

### Porous Particles

The respiratory tract encompasses the upper airways, including the oropharynx and larynx, followed by the lower airways, which include the trachea followed by bifurcations into the bronchi and bronchioli. The upper and lower airways are called the conducting airways. The terminal bronchioli then divide into respiratory bronchioli which then lead to the ultimate respiratory zone, the alveoli, or deep lung. Gonda, L. “Aerosols for delivery of therapeutic and diagnostic agents to the respiratory tract,” in Critical Reviews in Therapeutic Drug Carrier Systems 6.273-313, 1990. The deep lung, or alveoli, are the primary target of inhaled therapeutic aerosols for systemic drug delivery.

Accordingly, it can be important to deliver antiviral particles to the deep lung (i.e., the alveolar regions of the lung). Relatively large particles tend to get trapped in the oropharyngeal cavity, which can lead to excessive loss of the inhaled drug. Relatively smaller particles can be delivered to the deep lung, but can be phagocytosed. One way to deliver relatively large particles (sized to avoid phagocytosis), which are light enough to avoid excessive entrapment in the oropharyngeal cavity, is to use porous particles.

In one embodiment, the particles for delivering the compounds described herein to the alveolar regions of the lung are porous, “aerodynamically-light” particles, as described in U.S. Pat. No. 6,977,087. Aerodynamically light particles can be made of a biodegradable material, and typically have a tap density less than 0.4 g/cm³ and a mass mean diameter between 5 μm and 30 μm. The particles may be formed of biodegradable materials such as biodegradable polymers. For example, the particles may be formed of a functionalized polyester graft copolymer consisting of a linear alpha-hydroxy-acid polyester backbone having at least one amino acid group incorporated herein and at least one poly(amino acid) side chain extending from an amino acid group in the polyester backbone. In one embodiment, aerodynamically light particles having a large mean diameter, for example greater than 5 m, can be used for enhanced delivery of one or more of the compounds described herein to the alveolar region of the lung.

### Aqueous Solutions

Compounds can be administered intranasally, as well as topically, intranasally, intraveneously, by injection, and by nebulization, in aqueous solutions. In several embodiments, the solutions comprise one or more salts and are isotonic.

### Pressurized Containers

In certain embodiments, the disclosure contemplates a pressurized or unpressurized container comprising a compound or pharmaceutical composition as described herein. In certain embodiments, the container is a manual pump spray, inhaler, meter-dosed inhaler, dry powder inhaler, nebulizer, vibrating mesh nebulizer, jet nebulizer, or ultrasonic wave nebulizer.

In certain embodiments, a composition for inhalation comprises a compound disclosed herein and a propellant. In certain embodiments, the propellant is an aerosolizing propellant such as compressed air, ethanol, nitrogen, carbon dioxide, nitrous oxide, hydrofluoroalkanes (HFAs), 1,1,1,2,-tetrafluoroethane, 1,1,1,2,3,3,3-heptafluoropropane or combinations thereof.

### Nanoparticulate Compositions

The compounds described herein can also be administered in the form of nanoparticulate compositions. In one embodiment, controlled release nanoparticulate formulations comprise a nanoparticulate active agent to be administered and a rate-controlling polymer which prolongs the release of the agent following administration. In this embodiment, the compositions can release the active agent, following administration, for a time period ranging from about 2 to about 24 hours or up to 30 days or longer. Representative controlled release formulations including a nanoparticulate form of the active agent are described, for example, in U.S. Pat. No. 8,293,277.

Nanoparticulate compositions can comprise particles of the active agents described herein, having a non-crosslinked surface stabilizer adsorbed onto, or associated with, their surface.

The average particle size of the nanoparticulates is typically less than about 800 nm, more typically less than about 600 nm, still more typically less than about 400 nm, less than about 300 nm, less than about 250 nm, less than about 100 nm, or less than about 50 nm. In one aspect of this embodiment, at least 50% of the particles of active agent have an average particle size of less than about 800, 600, 400, 300, 250, 100, or 50 nm, respectively, when measured by light scattering techniques.

A variety of surface stabilizers are typically used with nanoparticulate compositions to prevent the particles from clumping or aggregating. Representative surface stabilizers are selected from the group consisting of gelatin, lecithin, dextran, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, polyoxyethylene stearates, colloidal silicon dioxide, phosphates, sodium dodecylsulfate, carboxymethylcellulose calcium, carboxymethylcellulose sodium, methylcellulose, hydroxyethylcellulose, hydroxypropylcellulose, hydroxypropylmethyl-cellulose phthalate, noncrystalline cellulose, magnesium aluminum silicate, triethanolamine, polyvinyl alcohol, polyvinylpyrrolidone, tyloxapol, poloxamers, poloxamines, poloxamine 908, dialkylesters of sodium sulfosuccinic acid, sodium lauryl sulfate, an alkyl aryl polyether sulfonate, a mixture of sucrose stearate and sucrose distearate, p-isononylphenoxypoly-(glycidol), SA90HCO, decanoyl-N-methylglucamide, n-decyl-D-glucopyranoside, n-decyl-D-maltopyranoside, n-dodecyl-D-glucopyranoside, n-dodecyl-D-maltoside, heptanoyl-N-methylglucamide, n-heptyl-D-glucopyranoside, n-heptyl-D-thioglucoside, n-hexyl-D-glucopyranoside, nonanoyl-N-methylglucamide, n-nonyl-D-glucopyranoside, octanoyl-N-methylglucamide, n-octyl-D-glucopyranoside, and octyl-D-thioglucopyranoside. Lysozymes can also be used as surface stabilizers for nanoparticulate compositions. Certain nanoparticles such as poly(lactic-co-glycolic acid) (PLGA)-nanoparticles are known to target the liver when given by intravenous (IV) or subcutaneously (SQ).

Representative rate controlling polymers into which the nanoparticles can be formulated include chitosan, polyethylene oxide (PEO), polyvinyl acetate phthalate, gum arabic, agar, guar gum, cereal gums, dextran, casein, gelatin, pectin, carrageenan, waxes, shellac, hydrogenated vegetable oils, polyvinylpyrrolidone, hydroxypropyl cellulose (HPC), hydroxyethyl cellulose (HEC), hydroxypropyl methylcelluose (HPMC), sodium carboxymethylcellulose (CMC), poly(ethylene) oxide, alkyl cellulose, ethyl cellulose, methyl cellulose, carboxymethyl cellulose, hydrophilic cellulose derivatives, polyethylene glycol, polyvinylpyrrolidone, cellulose acetate, cellulose acetate butyrate, cellulose acetate phthalate, cellulose acetate trimellitate, polyvinyl acetate phthalate, hydroxypropylmethyl cellulose phthalate, hydroxypropylmethyl cellulose acetate succinate, polyvinyl acetaldiethylamino acetate, poly(alkylmethacrylate), poly(vinyl acetate), polymers derived from acrylic or methacrylic acid and their respective esters, and copolymers derived from acrylic or methacrylic acid and their respective esters.

Methods of making nanoparticulate compositions are described, for example, in U.S. Pat. Nos. 5,518,187 and 5,862,999, both for “Method of Grinding Pharmaceutical Substances;” U.S. Pat. No. 5,718,388, for “Continuous Method of Grinding Pharmaceutical Substances;” and U.S. Pat. No. 5,510,118 for “Process of Preparing Therapeutic Compositions Containing Nanoparticles.”

Nanoparticulate compositions are also described, for example, in U.S. Pat. No. 5,298,262 for “Use of Ionic Cloud Point Modifiers to Prevent Particle Aggregation During Sterilization;” U.S. Pat. No. 5,302,401 for “Method to Reduce Particle Size Growth During Lyophilization;” U.S. Pat. No. 5,318,767 for “X-Ray Contrast Compositions Useful in Medical Imaging;” U.S. Pat. No. 5,326,552 for “Novel Formulation For Nanoparticulate X-Ray Blood Pool Contrast Agents Using High Molecular Weight Non-ionic Surfactants;” U.S. Pat. No. 5,328,404 for “Method of X-Ray Imaging Using Iodinated Aromatic Propanedioates;” U.S. Pat. No. 5,336,507 for “Use of Charged Phospholipids to Reduce Nanoparticle Aggregation;” U.S. Pat. No. 5,340,564 for Formulations Comprising Olin 10-G to Prevent Particle Aggregation and Increase Stability;” U.S. Pat. No. 5,346,702 for “Use of Non-Ionic Cloud Point Modifiers to Minimize Nanoparticulate Aggregation During Sterilization;” U.S. Pat. No. 5,349,957 for “Preparation and Magnetic Properties of Very Small Magnetic-Dextran Particles;” U.S. Pat. No. 5,352,459 for “Use of Purified Surface Modifiers to Prevent Particle Aggregation During Sterilization;” U.S. Pat. Nos. 5,399,363 and 5,494,683, both for “Surface Modified Anticancer Nanoparticles;” U.S. Pat. No. 5,401,492 for “Water Insoluble Non-Magnetic Manganese Particles as Magnetic Resonance Enhancement Agents;” U.S. Pat. No. 5,429,824 for “Use of Tyloxapol as a Nanoparticulate Stabilizer;” U.S. Pat. No. 5,447,710 for “Method for Making Nanoparticulate X-Ray Blood Pool Contrast Agents Using High Molecular Weight Non-ionic Surfactants;” U.S. Pat. No. 5,451,393 for “X-Ray Contrast Compositions Useful in Medical Imaging;” U.S. Pat. No. 5,466,440 for “Formulations of Oral Gastrointestinal Diagnostic X-Ray Contrast Agents in Combination with Pharmaceutically Acceptable Clays;” U.S. Pat. No. 5,470,583 for “Method of Preparing Nanoparticle Compositions Containing Charged Phospholipids to Reduce Aggregation;” U.S. Pat. No. 5,472,683 for “Nanoparticulate Diagnostic Mixed Carbamic Anhydrides as X-Ray Contrast Agents for Blood Pool and Lymphatic System Imaging;” U.S. Pat. No. 5,500,204 for “Nanoparticulate Diagnostic Dimers as X-Ray Contrast Agents for Blood Pool and Lymphatic System Imaging;” U.S. Pat. No. 5,518,738 for “Nanoparticulate NSAID Formulations;” U.S. Pat. No. 5,521,218 for “Nanoparticulate Iododipamide Derivatives for Use as X-Ray Contrast Agents;” U.S. Pat. No. 5,525,328 for “Nanoparticulate Diagnostic Diatrizoxy Ester X-Ray Contrast Agents for Blood Pool and Lymphatic System Imaging;” U.S. Pat. No. 5,543,133 for “Process of Preparing X-Ray Contrast Compositions Containing Nanoparticles;” U.S. Pat. No. 5,552,160 for “Surface Modified NSAID Nanoparticles;” U.S. Pat. No. 5,560,931 for “Formulations of Compounds as Nanoparticulate Dispersions in Digestible Oils or Fatty Acids;” U.S. Pat. No. 5,565,188 for “Polyalkylene Block Copolymers as Surface Modifiers for Nanoparticles;” U.S. Pat. No. 5,569,448 for “Sulfated Non-ionic Block Copolymer Surfactant as Stabilizer Coatings for Nanoparticle Compositions;” U.S. Pat. No. 5,571,536 for “Formulations of Compounds as Nanoparticulate Dispersions in Digestible Oils or Fatty Acids;” U.S. Pat. No. 5,573,749 for “Nanoparticulate Diagnostic Mixed Carboxylic Anydrides as X-Ray Contrast Agents for Blood Pool and Lymphatic System Imaging;” U.S. Pat. No. 5,573,750 for “Diagnostic Imaging X-Ray Contrast Agents;” U.S. Pat. No. 5,573,783 for “Redispersible Nanoparticulate Film Matrices WithProtective Overcoats;” U.S. Pat. No. 5,580,579 for “Site-specific Adhesion Within the GI Tract Using Nanoparticles Stabilized by High Molecular Weight, Linear Poly(ethylene Oxide) Polymers;” U.S. Pat. No. 5,585,108 for “Formulations of Oral Gastrointestinal Therapeutic Agents in Combination with Pharmaceutically Acceptable Clays;” U.S. Pat. No. 5,587,143 for “Butylene Oxide-Ethylene Oxide Block Copolymers Surfactants as Stabilizer Coatings for Nanoparticulate Compositions;” U.S. Pat. No. 5,591,456 for “Milled Naproxen with Hydroxypropyl Cellulose as Dispersion Stabilizer;” U.S. Pat. No. 5,593,657 for “Novel Barium Salt Formulations Stabilized by Non-ionic and Anionic Stabilizers;” U.S. Pat. No. 5,622,938 for “Sugar Based Surfactant for Nanocrystals;” U.S. Pat. No. 5,628,981 for “Improved Formulations of Oral Gastrointestinal Diagnostic X-Ray Contrast Agents and Oral Gastrointestinal Therapeutic Agents;” U.S. Pat. No. 5,643,552 for “Nanoparticulate Diagnostic Mixed Carbonic Anhydrides as X-Ray Contrast Agents for Blood Pool and Lymphatic System Imaging;” U.S. Pat. No. 5,718,388 for “Continuous Method of Grinding Pharmaceutical Substances;” U.S. Pat. No. 5,718,919 for “Nanoparticles Containing the R(−)Enantiomer of Ibuprofen;” U.S. Pat. No. 5,747,001 for “Aerosols Containing Beclomethasone Nanoparticle Dispersions;” U.S. Pat. No. 5,834,025 for “Reduction of Intravenously Administered Nanoparticulate Formulation Induced Adverse Physiological Reactions;” U.S. Pat. No. 6,045,829 “Nanocrystalline Formulations of Human Immunodeficiency Virus (HIV) Protease Inhibitors Using Cellulosic Surface Stabilizers;” U.S. Pat. No. 6,068,858 for “Methods of Making Nanocrystalline Formulations of Human Immunodeficiency Virus (HIV) Protease Inhibitors Using Cellulosic Surface Stabilizers;” U.S. Pat. No. 6,153,225 for “Injectable Formulations of Nanoparticulate Naproxen;” U.S. Pat. No. 6,165,506 for “New Solid Dose Form of Nanoparticulate Naproxen;” U.S. Pat. No. 6,221,400 for “Methods of Treating Mammals Using Nanocrystalline Formulations of Human Immunodeficiency Virus (HIV) Protease Inhibitors;” U.S. Pat. No. 6,264,922 for “Nebulized Aerosols Containing Nanoparticle Dispersions;” U.S. Pat. No. 6,267,989 for “Methods for Preventing Crystal Growth and Particle Aggregation in Nanoparticle Compositions;” U.S. Pat. No. 6,270,806 for “Use of PEG-Derivatized Lipids as Surface Stabilizers for Nanoparticulate Compositions;” U.S. Pat. No. 6,316,029 for “Rapidly Disintegrating Solid Oral Dosage Form,” U.S. Pat. No. 6,375,986 for “Solid Dose Nanoparticulate Compositions Comprising a Synergistic Combination of a Polymeric Surface Stabilizer and Dioctyl Sodium Sulfosuccinate;” U.S. Pat. No. 6,428,814 for “Bioadhesive nanoparticulate compositions having cationic surface stabilizers;” U.S. Pat. No. 6,431,478 for “Small Scale Mill;” and U.S. Pat. No. 6,432,381 for “Methods for targeting drug delivery to the upper and/or lower gastrointestinal tract,” all of which are specifically incorporated by reference. In addition, U.S. Patent Application No. 20020012675 A1, published on Jan. 31, 2002, for “Controlled Release Nanoparticulate Compositions,” describes nanoparticulate compositions, and is specifically incorporated by reference.

Amorphous small particle compositions are described, for example, in U.S. Pat. No. 4,783,484 for “Particulate Composition and Use Thereof as Antimicrobial Agent;” U.S. Pat. No. 4,826,689 for “Method for Making Uniformly Sized Particles from Water-Insoluble Organic Compounds;” U.S. Pat. No. 4,997,454 for “Method for Making Uniformly-Sized Particles From Insoluble Compounds;” U.S. Pat. No. 5,741,522 for “Ultrasmall, Non-aggregated Porous Particles of Uniform Size for Entrapping Gas Bubbles Within and Methods;” and U.S. Pat. No. 5,776,496, for “Ultrasmall Porous Particles for Enhancing Ultrasound Back Scatter.”

Certain nanoformulations can enhance the absorption of drugs by releasing drug into the lumen in a controlled manner, thus reducing solubility issues. The intestinal wall is designed to absorb nutrients and to act as a barrier to pathogens and macromolecules. Small amphipathic and lipophilic molecules can be absorbed by partitioning into the lipid bilayers and crossing the intestinal epithelial cells by passive diffusion, while nanoformulation absorption may be more complicated because of the intrinsic nature of the intestinal wall. The first physical obstacle to nanoparticle oral absorption is the mucus barrier which covers the luminal surface of the intestine and colon. The mucus barrier contains distinct layers and is composed mainly of heavily glycosylated proteins called mucins, which have the potential to block the absorption of certain nanoformulations. Modifications can be made to produce nanoformulations with increased mucus-penetrating properties (Ensign et al., “Mucus penetrating nanoparticles: biophysical tool and method of drug and gene delivery,” Adv Mater 24: 3887-3894 (2012)).

Once the mucus coating has been traversed, the transport of nanoformulations across intestinal epithelial cells can be regulated by several steps, including cell surface binding, endocytosis, intracellular trafficking and exocytosis, resulting in transcytosis (transport across the interior of a cell) with the potential involvement of multiple subcellular structures. Moreover, nanoformulations can also travel between cells through opened tight junctions, defined as paracytosis. Non-phagocytic pathways, which involve clathrin-mediated and caveolae-mediated endocytosis and macropinocytosis, are the most common mechanisms of nanoformulation absorption by the oral route.

Non-oral administration can provide various benefits, such as direct targeting to the desired site of action and an extended period of drug action. Transdermal administration has been optimized for nanoformulations, such as solid lipid nanoparticles (SLNs) and NEs, which are characterized by good biocompatibility, lower cytotoxicity and desirable drug release modulation (Cappel and Kreuter, “Effect of nanoparticles on transdermal drug delivery. J Microencapsul 8: 369-374 (1991)). Nasal administration of nanoformulations allows them to penetrate the nasal mucosal membrane, via a transmucosal route by endocytosis or via a carrier-or receptor-mediated transport process (Illum, “Nanoparticulate systems for nasal delivery of drugs: a real improvement over simple systems?” J. Pharm. Sci 96: 473-483 (2007)), an example of which is the nasal administration of chitosan nanoparticles of tizanidine to increase brain penetration and drug efficacy in mice (Patel et al., “Improved transnasal transport and brain uptake of tizanidine HCl-loaded thiolated chitosan nanoparticles for alleviation of pain,” J. Pharm. Sci 101: 690-706 (2012)). Pulmonary administration provides a large surface area and relative ease of access. The mucus barrier, metabolic enzymes in the tracheobronchial region and macrophages in the alveoli are typically the main barriers for drug penetration. Particle size is a major factor determining the diffusion of nanoformulation in the bronchial tree, with particles in the nano-sized region more likely to reach the alveolar region and particles with diameters between 1 and 5 m expected to deposit in the bronchioles (Musante et al., “Factors affecting the deposition of inhaled porous drug particles,” J Pharm Sci 91: 1590-1600 (2002)). A limit to absorption has been shown for larger particles, presumably because of an inability to cross the air-blood barrier. Particles can gradually release the drug, which can consequently penetrate into the blood stream or, alternatively, particles can be phagocytosed by alveolar macrophages (Bailey and Berkland, “Nanoparticle formulations in pulmonary drug delivery,” Med. Res. Rev., 29: 196-212 (2009)).

Certain nanoformulations have a minimal penetration through biological membranes in sites of absorption and for these, iv. administration can be the preferred route to obtain an efficient distribution in the body (Wacker, “Nanocarriers for intravenous injection—The long hard road to the market,” Int. J. Pharm., 457: 50-62, 2013).

The distribution of nanoformulations can vary widely depending on the delivery system used, the characteristics of the nanoformulation, the variability between individuals, and the rate of drug loss from the nanoformulations. Certain nanoparticles, such as solid drug nanoparticles (SDNs), improve drug absorption, which does not require them to arrive intact in the systemic circulation. Other nanoparticles survive the absorption process, thus altering the distribution and clearance of the contained drug.

Nanoformulations of a certain size and composition can diffuse in tissues through well-characterized processes, such as the enhanced permeability and retention effect, whereas others accumulate in specific cell populations, which allows one to target specific organs. Complex biological barriers can protect organs from exogenous compounds, and the blood-brain barrier (BBB) represents an obstacle for many therapeutic agents. Many different types of cells including endothelial cells, microglia, pericytes and astrocytes are present in the BBB, which exhibits extremely restrictive tight junctions, along with highly active efflux mechanisms, limiting the permeation of most drugs. Transport through the BBB is typically restricted to small lipophilic molecules and nutrients that are carried by specific transporters. One of the most important mechanisms regulating diffusion of nanoformulations into the brain is endocytosis by brain capillary endothelial cells.

Recent studies have correlated particle properties with nanoformulation entry pathways and processing in the human BBB endothelial barrier, indicating that uncoated nanoparticles have limited penetration through the BBB and that surface modification can influence the efficiency and mechanisms of endocytosis (Lee et al., “Targeting rat anti-mouse transferrin receptor monoclonal antibodies through blood-brain barrier in mouse,” J. Pharmacol. Exp. Ther. 292: 1048-1052 (2000)). Accordingly, surface-modified nanoparticles which cross the BBB, and deliver one or more of the compounds described herein, are within the scope of the invention.

Macrophages in the liver are a major pool of the total number of macrophages in the body. Kupffer cells in the liver possess numerous receptors for selective phagocytosis of opsonized particles (receptors for complement proteins and for the fragment crystallizable part of IgG). Phagocytosis can provide a mechanism for targeting the macrophages, and providing local delivery (i.e., delivery inside the macrophages) of the compounds described herein (TRUE?).

Nanoparticles linked to polyethylene glycol (PEG) have minimal interactions with receptors, which inhibits phagocytosis by the mononuclear phagocytic system (Bazile et al., “Stealth Me.PEG-PLA nanoparticles avoid uptake by the mononuclear phagocytes system,” J. Pharm. Sci. 84: 493-498 (1995)).

Representative nanoformulations include inorganic nanoparticles, SDNs, SLNs, NEs, liposomes, polymeric nanoparticles and dendrimers. The compounds described herein can be contained inside a nanoformulation, or, as is sometimes the case with inorganic nanoparticles and dendrimers, attached to the surface. Hybrid nanoformulations, which contain elements of more than one nanoformulation class, can also be used.

SDNs are lipid-free nanoparticles, which can improve the oral bioavailability and exposure of poorly water-soluble drugs (Chan, “Nanodrug particles and nanoformulations for drug delivery,” Adv. Drug. Deliv. Rev. 63: 405 (2011)). SDNs include a drug and a stabilizer, and are produced using ‘top-down’ (high pressure homogenization and wet milling) or bottom-up (solvent evaporation and precipitation) approaches.

SLNs consist of a lipid (or lipids) which is solid at room temperature, an emulsifier and water. Lipids utilized include, but are not limited to, triglycerides, partial glycerides, fatty acids, steroids and waxes. SLNs are most suited for delivering highly lipophilic drugs.

Liquid droplets of less than a 1000 nm dispersed in an immiscible liquid are classified as NEs. NEs are used as carriers for both hydrophobic and hydrophilic agents, and can be administered orally, transdermally, intravenously, intranasally, and ocularly. Oral administration can be preferred for chronic therapy, and NEs can effectively enhance oral bioavailability of small molecules, peptides and proteins.

Polymeric nanoparticles are solid particles typically around 200-800 nm in size, which can include synthetic and/or natural polymers, and can optionally be pegylated to minimize phagocytosis. Polymeric nanoparticles can increase the bioavailability of drugs and other substances, compared with traditional formulations. Their clearance depends on several factors, including the choice of polymers (including polymer size, polymer charge and targeting ligands), with positively charged nanoparticles larger than 100 nm being eliminated predominantly via the liver (Alexis et al., Factors affecting the clearance and biodistribution of polymeric nanoparticles. Mol Pharm 5: 505-515 (2008)).

Dendrimers are tree-like, nanostructured polymers which are commonly 10-20 nm in diameter.

Liposomes are spherical vesicles which include a phospholipid bilayer. A variety of lipids can be utilized, allowing for a degree of control in degradation level. In addition to oral dosing, liposomes can be administered in many ways, including intravenously (McCaskill et al., 2013), transdermally (Pierre and Dos Santos Miranda Costa, 2011), intravitreally (Honda et al., 2013) and through the lung (Chattopadhyay, 2013). Liposomes can be combined with synthetic polymers to form lipid-polymer hybrid nanoparticles, extending their ability to target specific sites in the body. The clearance rate of liposome-encased drugs is determined by both drug release and destruction of liposomes (uptake of liposomes by phagocyte immune cells, aggregation, pH-sensitive breakdown, etc.) (Ishida et al., “Liposome clearance,” Biosci Rep 22: 197-224 (2002)).

One of more of these nanoparticulate formulations can be used to deliver the active agents described herein to the macrophages, across the blood brain barrier, and other locations as appropriate.

### Controlled Release Formulations

In a preferred embodiment, the active compounds are prepared with carriers that will protect the compound against rapid elimination from the body, such as a controlled release formulation, including but not limited to implants and microencapsulated delivery systems. Biodegradable, biocompatible polymers can be used, such as ethylene vinyl acetate, polyanhydrides, polyglycolic acid, collagen, polyorthoesters and polylactic acid. For example, enterically coated compounds can be used to protect cleavage by stomach acid. Methods for preparation of such formulations will be apparent to those skilled in the art. Suitable materials can also be obtained commercially.

Liposomal suspensions (including but not limited to liposomes targeted to infected cells with monoclonal antibodies to viral antigens) are also preferred as pharmaceutically acceptable carriers. These can be prepared according to methods known to those skilled in the art, for example, as described in U.S. Pat. No. 4,522,811 (incorporated by reference). For example, liposome formulations can be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of the active compound is then introduced into the container. The container is then swirled by hand to free lipid material from the sides of the container and to disperse lipid aggregates, thereby forming the liposomal suspension.

### VIII. Combination Therapy

Depending on the specific RNA virus, the compounds described herein can be administered in combination or alternation with additional antiviral compounds. Further, where a viral infection causes inflammation, anti-inflammatory compounds can be administered, and where a secondary infection is present, or is to be prevented, an antibiotic can be administered. Additional types of compounds can also be administered, as discussed below, depending on the type of physiological damage the virus may cause, and/or the cytokine storm caused by the immune system may cause.

Representative additional active compounds include, but are not limited to, analgesics, anti-inflammatory drugs, antipyretics, antidepressants, antiepileptics, antihistamines, antimigraine drugs, antimuscarinics, anxioltyics, sedatives, hypnotics, antipsychotics, bronchodilators, anti-asthma drugs, cardiovascular drugs, corticosteroids, dopaminergics, electrolytes, gastro-intestinal drugs, muscle relaxants, nutritional agents, vitamins, parasympathomimetics, stimulants, anorectics, anti-narcoleptics, and antiviral agents. In a particular embodiment, the antiviral agent is a non-CNS targeting antiviral compound. “Adjunctive administration”, as used herein, means the compound can be administered in the same dosage form or in separate dosage forms with one or more other active agents. The additional active agent(s) can be formulated for immediate release, controlled release, or combinations thereof.

Specific examples of compounds that can be adjunctively administered with the compounds include, but are not limited to, aceclofenac, acetaminophen, adomexetine, almotriptan, alprazolam, amantadine, amcinonide, aminocyclopropane, amitriptyline, amolodipine, amoxapine, amphetamine, aripiprazole, aspirin, atomoxetine, azasetron, azatadine, beclomethasone, benactyzine, benoxaprofen, bermoprofen, betamethasone, bicifadine, bromocriptine, budesonide, buprenorphine, bupropion, buspirone, butorphanol, butriptyline, caffeine, carbamazepine, carbidopa, carisoprodol, celecoxib, chlordiazepoxide, chlorpromazine, choline salicylate, citalopram, clomipramine, clonazepam, clonidine, clonitazene, clorazepate, clotiazepam, cloxazolam, clozapine, codeine, corticosterone, cortisone, cyclobenzaprine, cyproheptadine, demexiptiline, desipramine, desomorphine, dexamethasone, dexanabinol, dextroamphetamine sulfate, dextromoramide, dextropropoxyphene, dezocine, diazepam, dibenzepin, diclofenac sodium, diflunisal, dihydrocodeine, dihydroergotamine, dihydromorphine, dimetacrine, divalproxex, dizatriptan, dolasetron, donepezil, dothiepin, doxepin, duloxetine, ergotamine, escitalopram, estazolam, ethosuximide, etodolac, femoxetine, fenamates, fenoprofen, fentanyl, fludiazepam, fluoxetine, fluphenazine, flurazepam, flurbiprofen, flutazolam, fluvoxamine, frovatriptan, gabapentin, galantamine, gepirone, ginko bilboa, granisetron, haloperidol, huperzine A, hydrocodone, hydrocortisone, hydromorphone, hydroxyzine, ibuprofen, imipramine, indiplon, indomethacin, indoprofen, iprindole, ipsapirone, ketaserin, ketoprofen, ketorolac, lesopitron, levodopa, lipase, lofepramine, lorazepam, loxapine, maprotiline, mazindol, mefenamic acid, melatonin, melitracen, memantine, meperidine, meprobamate, mesalamine, metapramine, metaxalone, methadone, methadone, methamphetamine, methocarbamol, methyldopa, methylphenidate, methylsalicylate, methysergid(e), metoclopramide, mianserin, mifepristone, milnacipran, minaprine, mirtazapine, moclobemide, modafmil (an anti-narcoleptic), molindone, morphine, morphine hydrochloride, nabumetone, nadolol, naproxen, naratriptan, nefazodone, neurontin, nomifensine, nortriptyline, olanzapine, olsalazine, ondansetron, opipramol, orphenadrine, oxaflozane, oxaprazin, oxazepam, oxitriptan, oxycodone, oxymorphone, pancrelipase, parecoxib, paroxetine, pemoline, pentazocine, pepsin, perphenazine, phenacetin, phendimetrazine, phenmetrazine, phenylbutazone, phenytoin, phosphatidylserine, pimozide, pirlindole, piroxicam, pizotifen, pizotyline, pramipexole, prednisolone, prednisone, pregabalin, propanolol, propizepine, propoxyphene, protriptyline, quazepam, quinupramine, reboxitine, reserpine, risperidone, ritanserin, rivastigmine, rizatriptan, rofecoxib, ropinirole, rotigotine, salsalate, sertraline, sibutramine, sildenafil, sulfasalazine, sulindac, sumatriptan, tacrine, temazepam, tetrabenozine, thiazides, thioridazine, thiothixene, tiapride, tiasipirone, tizanidine, tofenacin, tolmetin, toloxatone, topiramate, tramadol, trazodone, triazolam, trifluoperazine, trimethobenzamide, trimipramine, tropisetron, valdecoxib, valproic acid, venlafaxine, viloxazine, vitamin E, zimeldine, ziprasidone, zolmitriptan, zolpidem, zopiclone and isomers, salts, and combinations thereof.

In certain embodiments, the exemplary compounds and pharmaceutical compositions can be administered in combination with another antiviral agent(s) such as abacavir, acyclovir, acyclovir, adefovir, amantadine, amprenavir, ampligen, arbidol, atazanavir, atripla, balapiravir, BCX4430, boceprevir, cidofovir, combivir, daclatasvir, darunavir, dasabuvir, delavirdine, didanosine, docosanol, edoxudine, efavirenz, emtricitabine, enfuvirtide, entecavir, famciclovir, favipiravir, fomivirsen, fosamprenavir, foscamet, fosfonet, ganciclovir, GS-5734, ibacitabine, imunovir, idoxuridine, imiquimod, indinavir, inosine, interferon type III, interferon type II, interferon type I, lamivudine, ledipasvir, lopinavir, loviride, maraviroc, moroxydine, methisazone, nelfmavir, nevirapine, nexavir, NITD008, ombitasvir, oseltamivir, paritaprevir, peginterferon alfa-2a, penciclovir, peramivir, pleconaril, podophyllotoxin, raltegravir, ribavirin, rimantadine, ritonavir, pyramidine, saquinavir, simeprevir, sofosbuvir, stavudine, telaprevir, telbivudine, tenofovir, tenofovir disoproxil, Tenofovir Exalidex, tipranavir, trifluridine, trizivir, tromantadine, truvada, valaciclovir, valganciclovir, vicriviroc, vidarabine, viramidine zalcitabine, zanamivir, or zidovudine and combinations thereof.

Specific agents which can be used in combination, for use in treating or preventing specific RNA viral infections, are discussed in more detail below.

### Combination Therapy for Particular Use in Treating Covid-19 Infections

The compounds described herein can be combined with additional compounds useful for treating the disease states also treated by the release of NO. In particular, the compounds discussed below can be used in combination therapy to treat Covid-19 infections, or other respiratory infections with similar pathology.

Various compounds that can be combined with the compounds described herein are discussed below.

In one aspect of this embodiment, the at least one other active agent is selected from the group consisting of fusion inhibitors, entry inhibitors, protease inhibitors, polymerase inhibitors, antiviral nucleosides, such as remdesivir, GS-441524, N4-hydroxycytidine, and other compounds disclosed in U.S. Pat. No. 9,809,616, and their prodrugs, viral entry inhibitors, viral maturation inhibitors, JAK inhibitors, angiotensin-converting enzyme 2 (ACE2) inhibitors, SARS-CoV-specific human monoclonal antibodies, including CR3022, and agents of distinct or unknown mechanism.

Umifenovir (also known as Arbidol) is a representative fusion inhibitor.

Representative entry inhibitors include Camostat, luteolin, MDL28170, SSAA09E2, SSAA09E1 (which acts as a cathepsin L inhibitor), SSAA09E3, and tetra-O-galloyl-β-D-glucose (TGG). The chemical formulae of certain of these compounds are provided below:

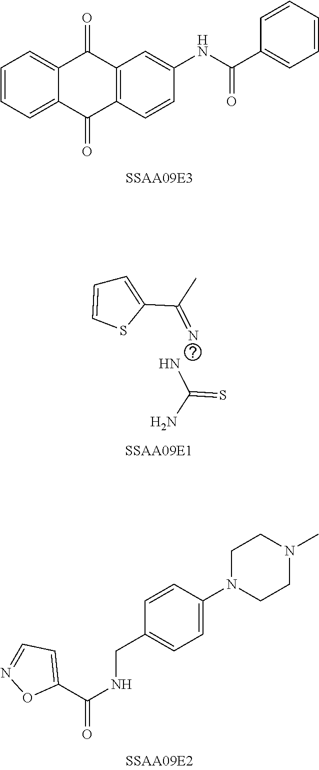

Other entry inhibitors include the following:

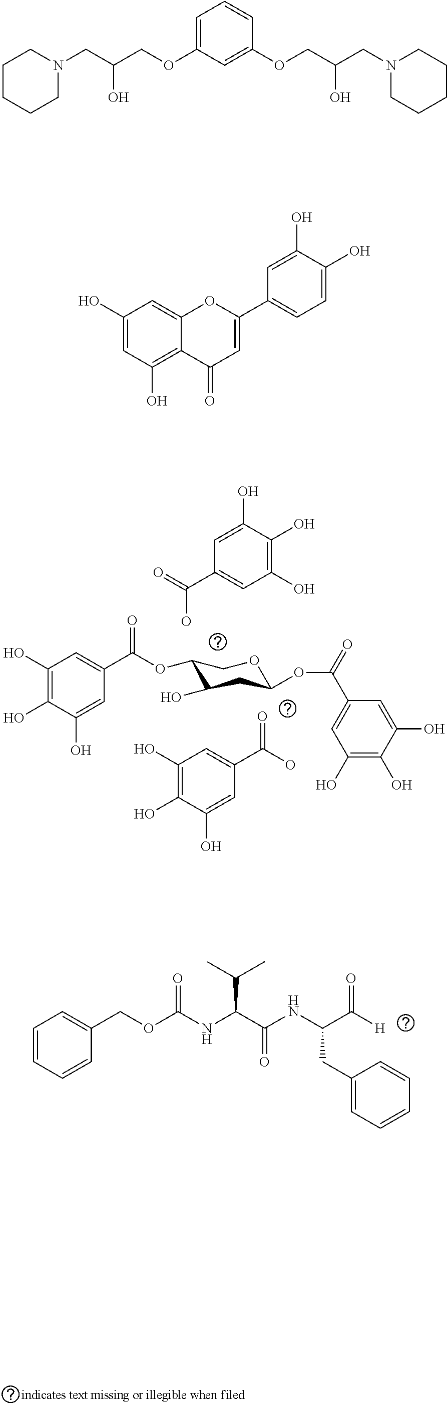

Remdesivir, Sofosbuvir, ribavirin, IDX-184 and GS-441524 have the following formulas:

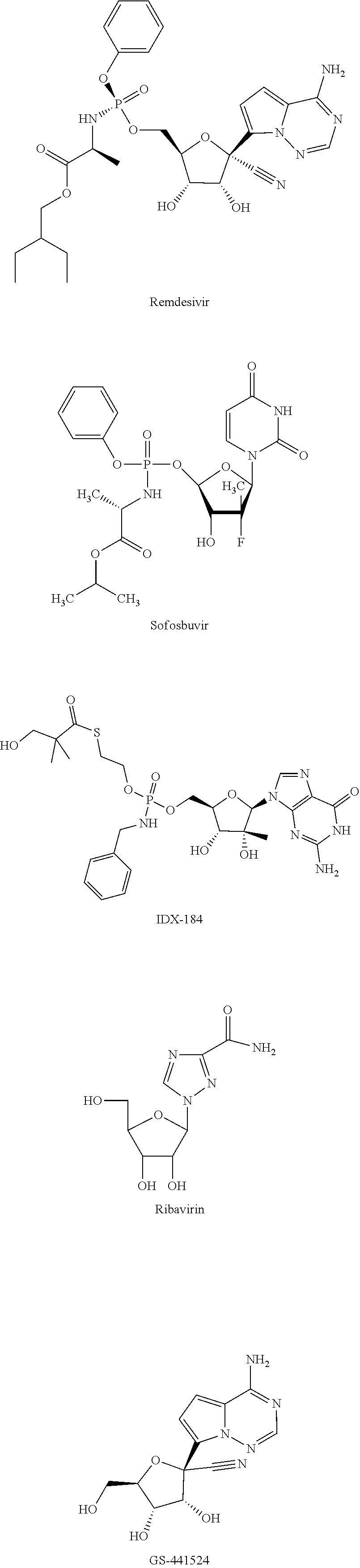

Additionally, one can administer compounds which inhibit the cytokine storm, such as dexamethasone, anti-coagulants and/or platelet aggregation inhibitors that address blood clots, or compounds which chelate iron ions released from hemoglobin by viruses such as COVID-19.

Representative ACE-2 inhibitors include sulfhydryl-containing agents, such as alacepril, captopril (capoten), and zefnopril, dicarboxylate-containing agents, such as enalapril (vasotec), ramipril (altace), quinapril (accupril), perindopril (coversyl), lisinopril (listril), benazepril (lotensin), imidapril (tanatril), trandolapril (mavik), and cilazapril (inhibace), and phosphonate-containing agents, such as fosinopril (fositen/monopril).

For example, when used to treat or prevent infection, the active compound or its prodrug or pharmaceutically acceptable salt can be administered in combination or alternation with another antiviral agent including, but not limited to, those of the formulae above. In general, in combination therapy, effective dosages of two or more agents are administered together, whereas during alternation therapy, an effective dosage of each agent is administered serially. The dosage will depend on absorption, inactivation and excretion rates of the drug, as well as other factors known to those of skill in the art. It is to be noted that dosage values will also vary with the severity of the condition to be alleviated. It is to be further understood that for any particular subject, specific dosage regimens and schedules should be adjusted over time according to the individual need and the professional judgment of the person administering or supervising the administration of the compositions.

A number of agents for combination with the compounds described herein are disclosed in Ghosh et al., “Drug Development and Medicinal Chemistry Efforts Toward SARS-Coronavirus and Covid-19 Therapeutics,” ChemMedChem 10.1002/cmdc.202000223.

Nonlimiting examples of antiviral agents that can be used in combination with the compounds disclosed herein include those listed below.

### Compounds for Inhibiting the Cytokine Storm

Throughout its activation, the inflammatory response must be regulated to prevent a damaging systemic inflammation, also known as a “cytokine storm.” A number of cytokines with anti-inflammatory properties are responsible for this, such as IL-10 and transforming growth factor β (TGF-β). Each cytokine acts on a different part of the inflammatory response. For example, products of the Th2 immune response suppress the Th1 immune response and vice versa.

By resolving inflammation, one can minimize collateral damage to surrounding cells, with little or no long-term damage to the patient. Accordingly, in addition to using the compounds described herein to inhibit the viral infection, one or more compounds which inhibit the cytokine storm can be co-administered.

Compounds which inhibit the cytokine storm include compounds that target fundamental immune pathways, such as the chemokine network and the cholinergic anti-inflammatory pathway.

JAK inhibitors, such as JAK 1 and JAK 2 inhibitors, can inhibit the cytokine storm, and in some cases, are also antiviral. Representative JAK inhibitors include those disclosed in U.S. Pat. No. 10,022,378, such as Jakafi, Tofacitinib, and Baricitinib, as well as LY3009104/INCB28050, Pacritinib/SB1518, VX-509, GLPG0634, INC424, R-348, CYT387, TG 10138, AEG 3482, and pharmaceutically acceptable salts and prodrugs thereof.

HMGB1 antibodies and COX-2 inhibitors can be used, which downregulate the cytokine storm. Examples of such compounds include Actemra (Roche). Celebrex (celecoxib), a COX-2 inhibitor, can be used. IL-8 (CXCL8) inhibitors can also be used.

Chemokine receptor CCR2 antagonists, such as PF-04178903 can reduce pulmonary immune pathology.

Selective α7Ach receptor agonists, such as GTS-21 (DMXB-A) and CNI-1495, can be used. These compounds reduce TNF-α. The late mediator of sepsis, HMGB1, downregulates IFN-γ pathways, and prevents the LPS-induced suppression of IL-10 and STAT 3 mechanisms.

### Compounds for Treating or Preventing Blood Clots

Viruses that cause respiratory infections, including Coronaviruses such as Covid-19, can be associated with pulmonary blood clots, and blood clots that can also do damage to the heart.

The compounds described herein can be co-administered with compounds that inhibit blood clot formation, such as blood thinners, or compounds that break up existing blood clots, such as tissue plasminogen activator (TPA), Integrilin (eptifibatide), abciximab (ReoPro) or tirofiban (Aggrastat).

Blood thinners prevent blood clots from forming, and keep existing blood clots from getting larger. There are two main types of blood thinners. Anticoagulants, such as heparin or warfarin (also called Coumadin), slow down biological processes for producing clots, and antiplatelet aggregation drugs, such as Plavix, aspirin, prevent blood cells called platelets from clumping together to form a clot.

By way of example, Integrilin® is typically administered at a dosage of 180 mcg/kg intravenous bolus administered as soon as possible following diagnosis, with 2 mcg/kg/min continuous infusion (following the initial bolus) for up to 96 hours of therapy.

Representative platelet aggregation inhibitors include glycoprotein IIB/IIIA inhibitors, phosphodiesterase inhibitors, adenosine reuptake inhibitors, and adenosine diphosphate (ADP) receptor inhibitors. These can optionally be administered in combination with an anticoagulant.

Representative anti-coagulants include coumarins (vitamin K antagonists), heparin and derivatives thereof, including unfractionated heparin (UFH), low molecular weight heparin (LMWH), and ultra-low-molecular weight heparin (ULMWH), synthetic pentasaccharide inhibitors of factor Xa, including Fondaparinux, Idraparinux, and Idrabiotaparinux, directly acting oral anticoagulants (DAOCs), such as dabigatran, rivaroxaban, apixaban, edoxaban and betrixaban, and antithrombin protein therapeutics/thrombin inhibitors, such as bivalent drugs hirudin, lepirudin, and bivalirudin and monovalent argatroban.

Representative platelet aggregation inhibitors include pravastatin, Plavix (clopidogrel bisulfate), Pletal (cilostazol), Effient (prasugrel), Aggrenox (aspirin and dipyridamole), Brilinta (ticagrelor), caplacizumab, Kengreal (cangrelor), Persantine (dipyridamole), Ticlid (ticlopidine), Yosprala (aspirin and omeprazole).

Additional Compounds that can be Used

Additional compounds and compound classes that can be used in combination therapy include the following: Antibodies, including monoclonal antibodies (mAb), Arbidol (umifenovir), Actemra (tocilizumab), APNO1 (Aperion Biologics), ARMS-1 (which includes Cetylpyridinium chloride (CPC)), ASC09 (Ascletis Pharma), AT-001 (Applied Therapeutics Inc.) and other aldose reductase inhibitors (ARI), ATYR1923 (aTyr Pharma, Inc.), Aviptadil (Relief Therapeutics), Azvudine, Bemcentinib, BLD-2660 (Blade Therapeutics), Bevacizumab, Brensocatib, Calquence (acalabrutinib), Camostat mesylate (a TMPRSS2 inhibitor), Camrelizumab, CAP-1002 (Capricor Therapeutics), CD₂₄Fcm, Clevudine, (Oncolmmune), CM4620-IE (CalciMedica Inc., CRAC channel inhibitor), Colchicine, convalescent plasma, CYNK-001 (Sorrento Therapeutics), DAS181 (Ansun Pharma), Desferal, Dipyridamole (Persantine), Dociparstat sodium (DSTAT), Duvelisib, Eculizumab, EIDD-2801 (Ridgeback Biotherapeutics), Emapalumab, Fadraciclib (CYC065) and seliciclib (roscovitine) (Cyclin-dependent kinase (CDK) inhibitors), Farxiga (dapagliflozin), Favilavir/Favipiravir/T-705/Avigan, Galidesivir, Ganovo (danoprevir), Gilenya (fingolimod) (sphingosine 1-phosphate receptor modulator), Gimsilumab, IFX-1, Ilaris (canakinumab), intravenous immunoglobulin, Ivermectin (importin α/β inhibitor), Kaletra/Aluvia (lopinavir/ritonavir), Kevzara (sarilumab), Kineret (anakinra), LAU-7b (fenretinide), Lenzilumab, Leronlimab (PRO 140), LY3127804 (an anti-Ang2 antibody), Leukine (sargramostim, a granulocyte macrophage colony stimulating factor), Losartan, Valsartan, and Telmisartan (Angiotensin II receptor antagonists), Meplazumab, Metablok (LSALT peptide, a DPEP1 inhibitor), Methylprednisolone and other corticosteroids, MN-166 (ibudilast, Macrophage migration inhibitory factor (MIF) inhibitor), MRx-4DP0004 (a strain of Bifidobacterium breve, 4D Pharma), Nafamostat (a serine protease inhibitor), Neuraminidase inhibitors like Tamiflu (oseltamivir), Nitazoxanide (nucleocapsid (N) protein inhibitor), Nivolumab, OT-101 (Mateon), Novaferon (man-made Interferon), Opaganib (yeliva) (Sphingosine kinase-2 inhibitor), Otilimab, PD-1 blocking antibody, peginterferons, such as peginterferon lambda, Pepcid (famotidine), Piclidenoson (A3 adenosine receptor agonist), Prezcobix (darunavir), PUL-042 (Pulmotect, Inc., toll-like receptor (TLR) binder), Rebif (interferon beta-1a), RHB-107 (upamostat) (serine protease inhibitor, RedHill Biopharma Ltd.), Selinexor (selective inhibitor of nuclear export (SINE)), SNG001 (Synairgen, inhaled interferon beta-1a), Solnatide, stem cells, including mesenchymal stem cells, MultiStem (Athersys), and PLX (Pluristem Therapeutics), Sylvant (siltuximab), Thymosin, TJM2 (TJ003234), Tradipitant (neurokinin-1 receptor antagonist), Truvada (emtricitabine and tenofovir), Ultomiris (ravulizumab-cwvz), Vazegepant (CGRP receptor antagonist or blocker), and Xofluza (baloxavir marboxil).

### Repurposed Antiviral Agents

A number of pharmaceutical agents, including agents active against other viruses, have been evaluated against Covid-19, and found to have activity. Any of these compounds can be combined with the compounds described herein. Representative compounds include lopinavir, ritonavir, niclosamide, promazine, PNU, UC2, cinanserin (SQ 10,643), Calmidazolium (C3930), tannic acid, 3-isotheaflavin-3-gallate, theaflavin-3,3′-digallate, glycyrrhizin, S-nitroso-N-acetylpenicillamine, nelfinavir, niclosamide, chloroquine, hydroxychloroquine, 5-benzyloxygramine, ribavirin, Interferons, such as Interferon (IFN)-α, IFN-β, and pegylated versions thereof, as well as combinations of these compounds with ribavirin, chlorpromazine hydrochloride, triflupromazine hydrochloride, gemcitabine, imatinib mesylate, dasatinib, and imatinib.

### Combination Therapy for Use With Flaviviruses

The compounds described herein, when used to treat a Flavivirus infection, such as dengue virus, yellow fever virus, West Nile virus or Japanese encephalitis virus, or infections caused by the Chikungunya virus (CHIKV), can be co-administered with any compound known to be useful for treating a disease vectored by biting insects, such as Zika virus, dengue virus, and yellow fever.

Therapeutic agents include, but are not limited to, chemotherapeutic agents, such as doxorubicin; dexamethasone; anti-infective agents, such as antibiotics (e.g. tetracycline, streptomycin, amphotericin and isoniazid), heavy metals such as antimony (e.g. pentavalent antimonials), anti-virals, anti-fungals, and anti-parasitics; immunological adjuvants; steroids; nucleotides, such as DNA, RNA, RNAi, siRNA, CpG or Poly (I:C); peptides; proteins; or metals such as silver, gallium or gadolinium, paromomycin, miltefosine, fluconazole, pentamide, Meglumine antimoniate, and combinations thereof.

In certain embodiments, the additional therapeutic agent is an antimicrobial drug selected from the group comprising: an antibiotic; an anti-tuberculosis antibiotic (such as isoniazid, streptamycin, or ethambutol); drugs with effect on Zika virus; drugs with effect on Dengue virus, drugs with effect on Yellow Fever, and drugs with effect on family Flaviviridae viruses. In certain embodiments, the therapeutic agent is an anti-microbial active, such as amoxicillin, ampicillin, tetracyclines, aminoglycosides (e.g., streptomycin), macrolides (e.g., erythromycin and its relatives), chloramphenicol, ivermectin, rifamycins and polypeptide antibiotics (e.g., polymyxin, bacitracin) and zwittermicin. In certain embodiments, the therapeutic agent is selected from isoniazid, doxorubicin, streptomycin, and tetracycline.

In other embodiments, the additional therapeutic agent is selected from the group consisting of cytostatic agents, alkylating agents, antimetabolites, anti-proliferative agents, tubulin binding agents, hormones and hormone antagonists, anthracycline drugs, vinca drugs, mitomycins, bleomycins, cytotoxic nucleosides, pteridine drugs, diynenes, podophyllotoxins, toxic enzymes, and radiosensitizing drugs. Representative therapeutic agents include mechlorethamine, triethylenephosphoramide, cyclophosphamide, ifosfamide, chlorambucil, busulfan, melphalan, triaziquone, nitrosourea compounds, adriamycin, carminomycin, daunorubicin (daunomycin), doxorubicin, isoniazid, indomethacin, gallium(III), 68gallium(III), aminopterin, methotrexate, methopterin, mithramycin, streptonigrin, dichloromethotrexate, mitomycin C, actinomycin-D, porfiromycin, 5-fluorouracil, floxuridine, ftorafur, 6-mercaptopurine, cytarabine, cytosine arabinoside, podophyllotoxin, etoposide, etoposide phosphate, melphalan, vinblastine, vincristine, leurosidine, vindesine, leurosine, taxol, taxane, cytochalasin B, gramicidin D, ethidium bromide, emetine, tenoposide, colchicin, dihydroxy anthracin dione, mitoxantrone, procaine, tetracaine, lidocaine, propranolol, puromycin, ricin subunit A, abrin, diptheria toxin, botulinum, cyanginosins, saxitoxin, shigatoxin, tetanus, tetrodotoxin, trichothecene, verrucologen, corticosteroids, progestins, estrogens, antiestrogens, androgens, aromatase inhibitors, calicheamicin, esperamicins, and dynemicins and combinations thereof.

In embodiments wherein the therapeutic agent is a hormone or hormone antagonist, representative additional therapeutic agents include prednisone, hydroxyprogesterone, medroprogesterone, diethylstilbestrol, tamoxifen, testosterone, and aminogluthetimide and combinations thereof.

Additional compounds that can be co-administered include phosphate-containing prodrugs, thiophosphate-containing prodrugs, sulfate containing prodrugs, peptide containing prodrugs, (-lactam-containing prodrugs, optionally substituted phenoxyacetamide-containing prodrugs, optionally substituted phenylacetamide-containing prodrugs, 5-fluorocytosinem, and 5-fluorouridine prodrugs that can be converted to the more active cytotoxic free drug and combinations thereof.

Another example of anti-Flaviviridae compounds are retinoic acid analogues, such as those disclosed in U.S. Pat. No. 11,007,160. Retinoids are a class of natural and synthetic vitamin A-derivatives in which the terminal carboxyl group of retinoic acid is linked to an aminophenol residue. Representative retinoic acid analogues include N-(4-hydroxyphenyl) retinamide (4-HPR), also known as Fenretinide, and 4-HPR metabolites, such as N-(4-hydroxyphenyl)-4-oxoretinamide (4-oxo-4-HPR).

NSAIDS and other suitable pain relievers can also be used to help treat the associated symptoms of certain Flaviridae viruses, including DENV, YFV, WNV, or JEV infection.

Celgosivir and analogs of celgosivir disclosed in U.S. Pat. No. 11,000,516 can also be co-administered.

The compounds described herein can also be administered in combination with a protease inhibitor, NS5A inhibitor, polymerase inhibitor, ribavirin, interferon, an RNA helicase DDX3 inhibitor, such as those disclosed in U.S. Pat. No. 10,941,121, a mono- or di-substituted indole or substituted indolene Dengue viral replication inhibitors, such as those disclosed in U.S. Pat. Nos. 10,919,854 and 10,913,716, an adaptor-associated kinase 1 (AAK1) inhibitor or cyclin G-associated kinase (GAK) inhibitor, such as sunitinib, erlotinib, PKC-412 or midostaurin, toll-like receptor (TLR) agonists, or P-selectin glycoprotein ligand-1 (PSGL-1) agonists or antagonists.

In one embodiment, the deuterated compounds described herein are combined with protease inhibitors or other non-C or U nucleoside antiviral analogs.

The present invention will be better understood with reference to the following non-limiting examples.

### Example 1

Conversion of NHC to 2′-deoxy-NHC-TP

N⁴-hydroxycytidine (NHC) is active against coronaviruses, as shown in the following table:

| | Anti-SARS-Cov-2 | Anti-Hcov-OC43 |
|---|---|---|
| EC_{50/90} (μM) | 0.3/0.8 | 0.8/1.8 |

| | PBM | CEM | Vero | Huh7 |
|---|---|---|---|---|
| CC₅₀ (μM) | 44.3 | 3.5 | 12.6 | 80.3 |

However, N⁴-hydroxycytidine is known to be mutagenic (see, for example, Celina Janion, Barry W. Glickman, “N4-hydroxycytidine: A mutagen specific for at to GC transitions, Mutation Research/Fundamental and Molecular Mechanisms of Mutagenesis,” Volume 72, Issue 1, pages 43-47 (1980)).

It is believed that N⁴-hydroxycytidine (NHC) can be converted to 2′-deoxy N⁴-hydroxycytidine triphosphate (2′ deoxy NHC-TP) in cells, which can induce genotoxicity. More specifically, 2′-deoxy N⁴-hydroxycytidine diphosphate (NHC-DP) can be converted to 2′ deoxy NHC-DP by RNR, which can be phosphorylated to 2′ deoxy NHC-TP, that can be incorporated by cellular DNA polymerases, which in turn induces genotoxicity.

When the compounds described herein are administered to a patient infected with an RNA virus, a portion of the drug is utilized for its intended purpose, i.e., inactivation of RNA viruses, and a portion is excreted, in its original form, or as one or more metabolites.

While not wishing to be bound to a particular theory, it is believed that the deuterated and/or methylated compounds described herein will delay metabolism at the 3′-position, i.e., delay the conversion of the compounds described herein to deoxyribonucleotide analogs. By delaying this metabolism, the compounds can be incorporated into the RNA of the RNA viruses, and all or most of the remaining compound that is not incorporated into the RNA of the RNA viruses is excreted or metabolized via a different pathway before being converted to the deoxyribonucleic acid analog, and incorporated by cellular DNA polymerases, which in turn reduces or avoids genotoxicity.

Ribonucleotide reductase (RNR), also known as ribonucleoside diphosphate reductase (rNDP), is an enzyme that catalyzes the formation of deoxyribonucleotides from ribonucleotides.

This example shows an RNR assay to follow the production of deoxy-NHC-DP over time, and a cell culture assay to follow the production of deoxy-NHC, -MP, -DP, and-TP over time. The RNR assay was conducted, according to the techniques described in Brignole et al., eLife 2018; 7:e31502 doi: 10.7554/eLife.31502.

### Conditions:

1) rCDP (rCytidine diphosphate), no RNR (water instead)
2-4) rCDP+RNR
5) NHC-DP, no RNR (water instead)
6-8) NHC-DP+RNR

### Reactions:

2 μL 5× RNR Reaction Buffer
1 μL 15 μM M1 (in RNR storage buffer)
2 μL 35 μM freshly reconstituted M2 (In RNR storage buffer)
2.9 μL 1 mg/mL human thioredoxin
0.3 μL 0.5 mg/mL human thioredoxin reductase
1 μL 25 mM substrate (rCDP or NHC-DP)
0.8 μL MQ water
Total 10 μL Reactions in PCR tubes

The following table shows the components of the RNR Reaction Buffer:

RNR 5x Reaction Buffer

| | | |
|---|---|---|
| Components | Final Conc. | Dilute To |

| | | | | | | |
|---|---|---|---|---|---|---|
| HEPES pH 7.6 | 104.2 | ul | 250 | mM | 50 | mM |
| KCl | 11.2 | mg | 750 | mM | 150 | mM |
| MgCl₂ | 3 | mg | 75 | mM | 15 | mM |
| ATP | 30 | ul | 15 | mM | 3 | mM |
| NADPH | 1.7 | mg | 10 | mM | 2 | mM |
| Total Volume | 200 | ul | | | | |

Five Master Mix Reactions were prepared per substrate, and 7 μL was aliquoted from the Master Mix into four separate epi tubes. For a negative control, 3 μL of water was added. RNR was added as described above for reactions.

The mixtures were incubated at 37° C. for 1 hr in a Thermocycler, then heat inactivated at 95° C. for 3 min in the Thermocycler. The samples were diluted two hundred and fifty fold (250×) and then subjected to LC-MS/MS analysis to follow the loss of rCDP, and the gain of 2′ deoxy NHC-TP, over time.

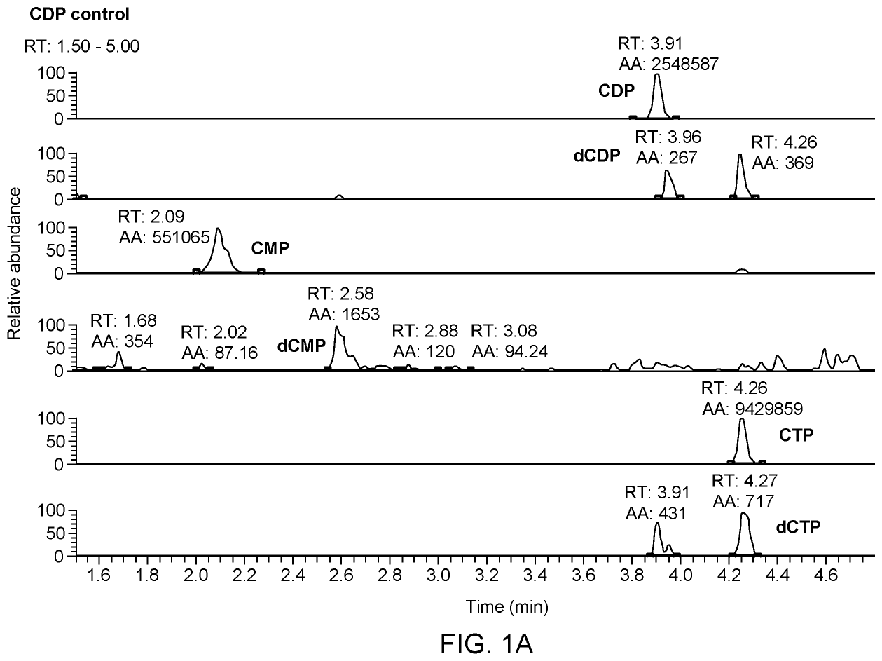

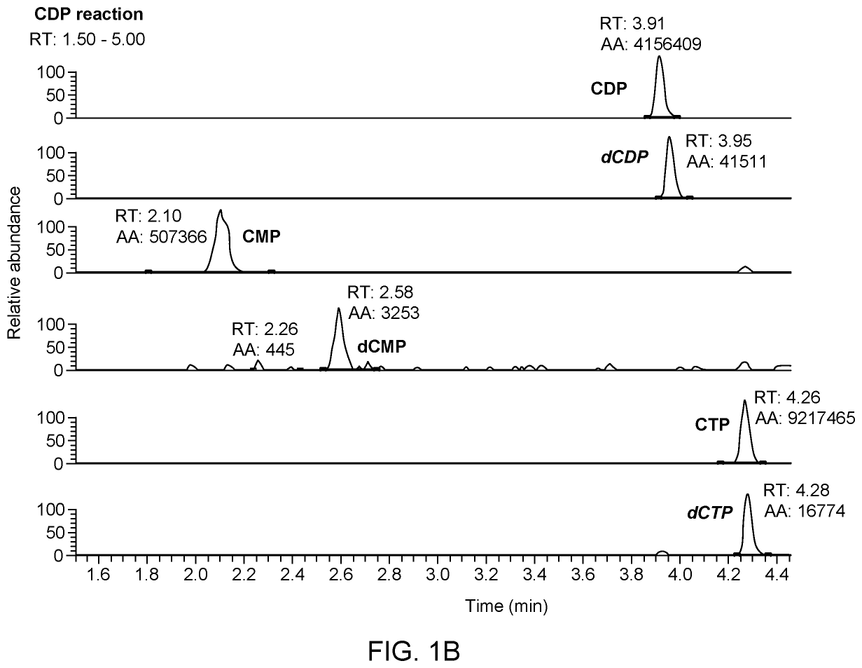

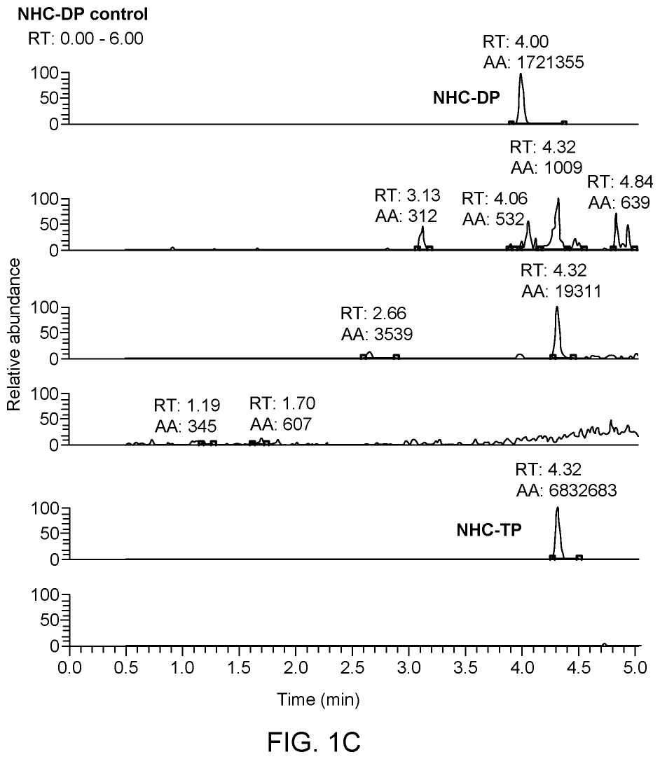

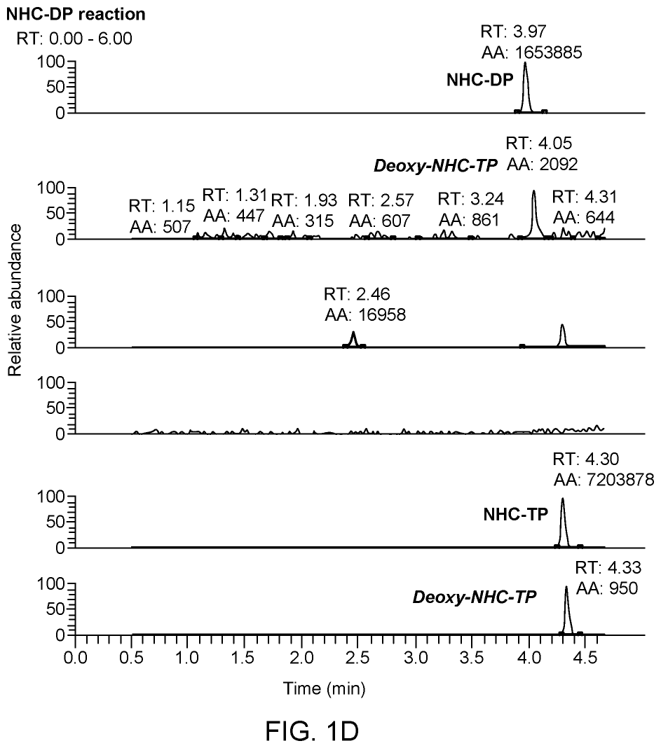

The results are shown in FIGS. 1A-D, where FIG. 1A shows a CDP control, FIG. 1B shows the CDP reaction, FIG. 1C shows the NHC-DP control, and FIG. 1D shows the NHC-DP reaction.

Besides CDP/dCDP or NHC-DP/deoxy-NHC-DP, CTP/dCTP or NHC-TP/deoxy-NHC-TP were also detected. ADP and AMP were detected in all of the samples, including the control sample. ATP as a phosphate donor, played the role in converting CDP to CTP and NHC-DP to NHC-TP.

This experiment shows that RNR converted a portion of the NCH-DP to dNHC-TP.

### Incubation of NHC in Vero Cells

A further experiment was conducted to show what occurs when NHC is incubated in Vero cells. A 12-well plate was seeded with 1 million vero cells/well. Either 10 μM or 50 μM NHC was added to each well, and the plate was incubated for 4 h at 37° C.

The NHC derivatives were measured in both supernatant and cell pellet using LC-MS/MS. The LC-MS/MS instrument was a Thermo TSQ Quantiva, equipped with a Kinetex EVO-C18 (100×2.1 mm, 2.6 μm) column.

The buffers used for the liquid chromatography were as follows:

For cell pellet extractions: A) 2 mM ammonium phosphate and 3 mM hexylamine, B) acetonitrile.

For supernatant: A) 0.1% formic acid, B) acetonitrile.

MS: MRM mode, both positive and negative, monitoring for NHC, deoxy-NHC, and their respective monophosphates (-MP), diphosphates (-DP) and triphosphates (-TP).

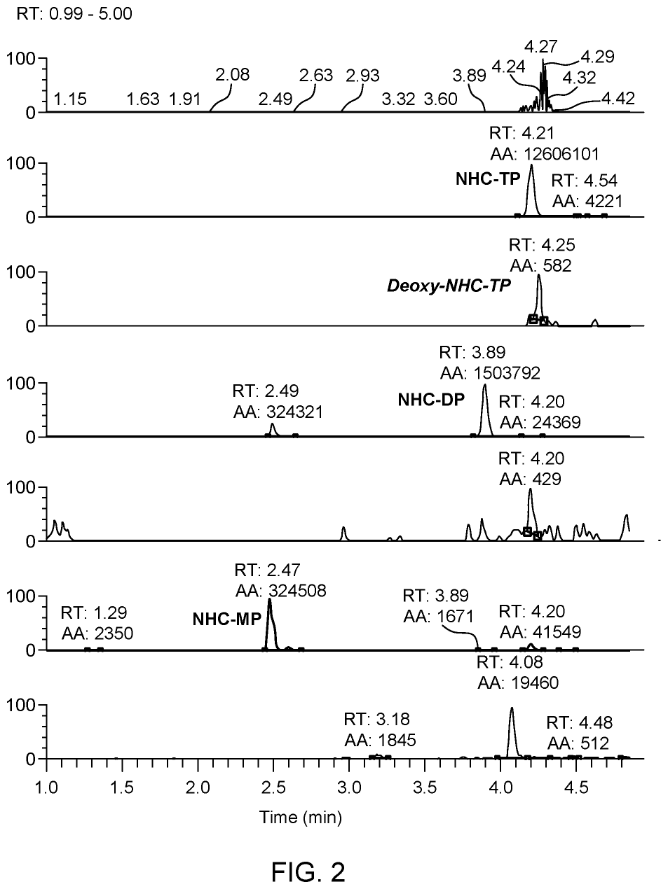

The results are shown in FIG. 2. As shown in FIG. 2, CDP was converted to dCDP by RNR, with the ratio in the samples around 100:0.7 (CDP: dCDP), and 100:0.1 (CTP:dCTP), calculated by comparing peak areas. NHC-DP was converted to deoxy-NHC-DP by RNR with a much lower efficiency than CDP to dCDP. The ratio in the samples was around 100:0.1 (NHC-DP: deoxy-NHC-DP) and 100:0.009 (NHC-TP: deoxy-NHC-TP).

The conversion percentage could not be calculated, since a relatively large amount of CDP or NHC-DP was phosphorylated to CTP or NHC-TP by the reaction system, even in the control samples.

In vero cells, a trace amount of deoxy-NHC-TP was detected in 50 μM NHC treated samples, but not in 10 μM NHC treated samples. The level of deoxy-NHC, -MP and-DP were below limit of detection.

In vero cell medium, no deoxy-NHC or its derivatives was detected.

### Example 2

### Cellular Toxicity Assays

The toxicity of the compounds can be assessed in Vero, human PBM, CEM (human lymphoblastoid), MT-2, and HepG2 cells, as described previously (see Schinazi R. F., Sommadossi J.-P., Saalmann V., Cannon D. L., Xie M.-Y., Hart G. C., Smith G. A. & Hahn E. F. Antimicrob. Agents Chemother. 1990, 34, 1061-67). Cycloheximide can be included as positive cytotoxic control, and untreated cells exposed to solvent can be included as negative controls. The cytotoxicity IC₅₀ can be obtained from the concentration-response curve using the median effective method described previously (see Chou T.-C. & Talalay P. Adv. Enzyme Regul. 1984, 22, 27-55; Belen'kii M. S. & Schinazi R. F. Antiviral Res. 1994, 25, 1-11).

### Example 3

### Mitochondrial Toxicity Assays in HepG2 Cells:

i) Effect of Compounds on Cell Growth and Lactic Acid Production: The effect on the growth of HepG2 cells can be determined by incubating cells in the presence of 0 μM, 0.1 μM, 1 μM, 10 μM and 100 μM drug. Cells (5×10⁴ per well) can be plated into 12-well cell culture clusters in minimum essential medium with nonessential amino acids supplemented with 10% fetal bovine serum, 1% sodium pyruvate, and 1% penicillin/streptomycin and incubated for 4 days at 37° C. At the end of the incubation period the cell number can be determined using a hemocytometer. Also taught by Pan-Zhou X-R, Cui L, Zhou X-J, Sommadossi J-P, Darley-Usmer V M. “Differential effects of antiretroviral nucleoside analogs on mitochondrial function in HepG2 cells,” Antimicrob. Agents Chemother. 2000; 44: 496-503.

To measure the effects of the compounds on lactic acid production, HepG2 cells from a stock culture can be diluted and plated in 12-well culture plates at 2.5×10⁴ cells per well. Various concentrations (0 RM, 0.1 μM, 1 μM, 10 μM and 100 μM) of compound can be added, and the cultures can be incubated at 37° C. in a humidified 5% CO₂ atmosphere for 4 days. At day 4, the number of cells in each well can be determined and the culture medium collected. The culture medium can then be filtered, and the lactic acid content in the medium determined using a colorimetric lactic acid assay (Sigma-Aldrich). Since lactic acid product can be considered a marker for impaired mitochondrial function, elevated levels of lactic acid production detected in cells grown in the presence of test compounds indicates a drug-induced cytotoxic effect.

ii) Effect on Compounds on Mitochondrial DNA Synthesis: a real-time PCR assay to accurately quantify mitochondrial DNA content has been developed (see Stuyver L J, Lostia S, Adams M, Mathew J S, Pai B S, Grier J, Tharnish P M, Choi Y, Chong Y, Choo H, Chu C K, Otto M J, Schinazi R F. Antiviral activities and cellular toxicities of modified 2′,3′-dideoxy-2′,3′-didehydrocytidine analogs. Antimicrob. Agents Chemother. 2002; 46: 3854-60). This assay can be used in all studies described in this application that determine the effect of compounds on mitochondrial DNA content. In this assay, low-passage-number HepG2 cells are seeded at 5,000 cells/well in collagen-coated 96-well plates. Test compounds are added to the medium to obtain final concentrations of 0 μM, 0.1 μM, 10 μM and 100 μM. On culture day 7, cellular nucleic acids can be prepared by using commercially available columns (RNeasy 96 kit; Qiagen). These kits co-purify RNA and DNA, and hence, total nucleic acids are eluted from the columns. The mitochondrial cytochrome c oxidase subunit II (COXII) gene and the ß-actin or rRNA gene can be amplified from 5 μl of the eluted nucleic acids using a multiplex Q-PCR protocol with suitable primers and probes for both target and reference amplifications. For COXII the following sense, probe and antisense primers can be used, respectively: 5′-TGCCCGCCATCATCCTA-3′, 5′-tetrachloro-6-carboxyfluorescein-TCCTCATCGCCCTCCCATCCC-TAMRA-3′ and 5′-CGTCTGTTATGTAAAGGATGCGT-3′. For exon 3 of the ß-actin gene (GenBank accession number E01094) the sense, probe, and antisense primers are 5′-GCGCGGCTACAGCTTCA-3′, 5′-6-FAMCACCACGGCCGAGCGGGATAMRA-3′ and 5′-TCTCCTTAATGTCACGCACGAT-3′, respectively. The primers and probes for the rRNA gene are commercially available from Applied Biosystems. Since equal amplification efficiencies are obtained for all genes, the comparative CT method can be used to investigate potential inhibition of mitochondrial DNA synthesis. The comparative CT method uses arithmetic formulas in which the amount of target (COXII gene) is normalized to the amount of an endogenous reference (the ß-actin or rRNA gene) and is relative to a calibrator (a control with no drug at day 7). The arithmetic formula for this approach is given by 2-ΔACT, where ΔACT is (CT for average target test sample—CT for target control)—(CT for average reference test-CT for reference control) (see Johnson M R, K Wang, JB Smith, MJ Heslin, RB Diasio. Quantitation of dihydropyrimidine dehydrogenase expression by real-time reverse transcription polymerase chain reaction. Anal. Biochem. 2000; 278:175-184). A decrease in mitochondrial DNA content in cells grown in the presence of drug indicates mitochondrial toxicity.

### Example 4

### Mitochondrial Toxicity—Glu/Gal

### Protocol Summary

HepG2 cells can be plated on 96 or 384 well tissue culture polystyrene plates. After 24 hr the cells can be dosed with test compound at a range of concentrations and incubated for 72 hr in medium supplemented with either galactose or glucose. Test compounds are said to cause mitochondrial toxicity if the cells grown in galactose-containing medium are more sensitive to the test compound than the cells grown in glucose-containing medium.

Objective: To measure the sensitivity of HepG2 cells grown in medium containing either galactose or glucose to the test compound.

### Experimental Procedure

HepG2 human hepatocellular carcinoma cells are plated on 96 or 384-well tissue culture polystyrene plates containing either galactose or glucose containing medium supplemented with 10% fetal bovine serum and antibiotics and incubated overnight. The cells are dosed with increasing concentrations of the test compound (final DMSO concentration 0.5%; typical final test compound concentrations of 100, 30, 10, 3, 1, 0.3, 0.1, 0.03 μM for an eight point dose response curve; n=3 replicates per concentration) and the cells are incubated for 72 hr. Appropriate controls are simultaneously used as quality controls. Cell viability is measured using Hoechst staining and cell counting by a HCS reader.

### Example 5

### Mitochondrial Toxicity Assays in Neuro2A Cells

To estimate the potential of the compounds described herein to cause neuronal toxicity, mouse Neuro2A cells (American Type Culture Collection 131) can be used as a model system (see Ray A S, Hernandez-Santiago B I, Mathew J S, Murakami E, Bozeman C, Xie M Y, Dutschman G E, Gullen E, Yang Z, Hurwitz S, Cheng Y C, Chu C K, McClure H, Schinazi R F, Anderson K S. Mechanism of anti-human immunodeficiency virus activity of beta-D-6-cyclopropylamino-2′,3′-didehydro-2′,3′-dideoxyguanosine. Antimicrob. Agents Chemother. 2005, 49, 1994-2001). The concentrations necessary to inhibit cell growth by 50% (CC50) can be measured using the 3-(4,5-dimethyl-thiazol-2-yl)-2,5-diphenyltetrazolium bromide dye-based assay, as described. Perturbations in cellular lactic acid and mitochondrial DNA levels at defined concentrations of drug can be carried out as described above. ddC and AZT can be used as control nucleoside analogs.

### Example 6

### Assay for Bone Marrow Cytotoxicity

Primary human bone marrow mononuclear cells can be obtained commercially from Cambrex Bioscience (Walkersville, MD). CFU-GM assays is carried out using a bilayer soft agar in the presence of 50 units/mL human recombinant granulocyte/macrophage colony-stimulating factor, while BFU-E assays used a ethylcellulose matrix containing 1 unit/mL erythropoietin (see Sommadossi J P, Carlisle R. Toxicity of 3′-azido-3′-deoxythymidine and 9-(1,3-dihydroxy-2-propoxymethyl) guanine for normal human hepatopoietic progenitor cells in vitro. Antimicrob. Agents Chemother. 1987; 31: 452-454; Sommadossi, JP, Schinazi, RF, Chu, C K, and Xie, MY. Comparison of cytotoxicity of the (−) and (+) enantiomer of 2′,3′-dideoxy-3′-thiacytidine in normal human bone marrow progenitor cells. Biochem. Pharmacol. 1992; 44:1921-1925). Each experiment can be performed in duplicate in cells from three different donors. AZT is used as a positive control. Cells can be incubated in the presence of the compound for 14-18 days at 37° C. with 5% CO₂, and colonies of greater than 50 cells can be counted using an inverted microscope to determine the IC₅₀. The 50% inhibitory concentration (IC₅₀) can be obtained by least-squares linear regression analysis of the logarithm of drug concentration versus BFU-E survival fractions. Statistical analysis can be performed with Student's t test for independent non-paired samples.

### Example 7

### In Vitro Human Mitochondrial RNA Polymerase (POLRMT) Assay

In vitro RNA nucleotide incorporation assays with POLRMT (INDIGO Biosciences) can be performed as previously described (Arnold et al. 2012). Briefly, ³²P-radiolabeled RNA primer (5′-UUUUGCCGCGCC) can be hybridized to 3 molar excess of the appropriate DNA template (5′-GGGAATGCANGGCGCGGC where position N can be replaced by A, T, or C). 125 nM of POLRMT can be incubated with 500 nM of 5′-radiolabled RNAIDNA hybrid, 10 mM MgCl₂ and 100 μM of the corresponding nucleoside triphosphate. For non-nucleoside analogs, 100 μM of inhibitor can be added at the same time as 100 μM UTP. Incorporation can be allowed to proceed for 2 h at 30° C. and reactions are stopped by the addition of 10 mM EDTA and formamide. Samples are visualized on 20% denaturing polyacrylamide gel. Data can be analyzed by normalizing the product fraction for each nucleoside triphosphate analog to that of the corresponding natural nucleoside triphosphate.

### Example 8

### Effect of Nucleotide Analogs on the DNA Polymerase and Exonuclease Activities of Mitochondrial DNA Polymerase γ

i) Purification of Human Polymerase γ: The recombinant large and small subunits of polymerase γ can be purified as described previously (see Graves S W, Johnson A A, Johnson K A. Expression, purification, and initial kinetic characterization of the large subunit of the human mitochondrial DNA polymerase. Biochemistry. 1998, 37, 6050-8; Johnson A A, Tsai Y, Graves S W, Johnson K A. Human mitochondrial DNA polymerase holoenzyme: reconstitution and characterization. Biochemistry 2000; 39: 1702-8). The protein concentration can be determined spectrophotometrically at 280 nm, with extinction coefficients of 234,420, and 71,894 M-1 cm-1 for the large and the small subunits of polymerase γ, respectively.

ii) Kinetic Analyses of Nucleotide Incorporation: Pre-steady-state kinetic analyses can be performed to determine the catalytic efficiency of incorporation (k/K) for DNA polymerase γ for nucleoside-TP and natural dNTP substrates. This allowed determination of the relative ability of this enzyme to incorporate modified analogs and predict toxicity. Pre-steady-state kinetic analyses of incorporation of nucleotide analogs by DNA polymerase 7 would be carried out essentially as described previously (see Murakami E, Ray A S, Schinazi R F, Anderson K S. Investigating the effects of stereochemistry on incorporation and removal of 5-fluorocytidine analogs by mitochondrial DNA polymerase gamma: comparison of D- and L-D4FC-TP. Antiviral Res. 2004, 62, 57-64; Feng J Y, Murakami E, Zorca S M, Johnson A A, Johnson K A, Schinazi R F, Furman P A, Anderson K S. Relationship between antiviral activity and host toxicity: comparison of the incorporation efficiencies of 2′,3′-dideoxy-5-fluoro-3′-thiacytidine-triphosphate analogs by human immunodeficiency virus type 1 reverse transcriptase and human mitochondrial DNA polymerase. Antimicrob Agents Chemother. 2004, 48, 1300-6). Briefly, a pre-incubated mixture of large (250 nM) and small (1.25 mM) subunits of polymerase γ and 60 nM DNA template/primer in 50 mM Tris-HCl, 100 mM NaCl, pH 7.8, can be added to a solution containing MgCl₂ (2.5 mM) and various concentrations of nucleotide analogs. Reactions can be quenched and analyzed as described previously. Data can be fit to the same equations as described above.

iii) Assay for Human Polymerase γ 3′ 5′ Exonuclease Activity: The human polymerase γ exonuclease activity can be studied by measuring the rate of formation of the cleavage products in the absence of dNTP. The reaction can be initiated by adding MgCl₂ (2.5 mM) to a pre-incubated mixture of polymerase γ large subunit (40 nM), small subunit (270 nM), and 1,500 nM chain-terminated template/primer in 50 mM Tris-HCl, 100 mM NaCl, pH 7.8, and quenched with 0.3M EDTA at the designated time points. All reaction mixtures would be analyzed on 20% denaturing polyacrylamide sequencing gels (8M urea), imaged on a Bio-Rad GS-525 molecular image system, and quantified with Molecular Analyst (Bio-Rad). Products formed from the early time points would be plotted as a function of time. Data would be fitted by linear regression with Sigma Plot (Jandel Scientific). The slope of the line can be divided by the active enzyme concentration in the reaction to calculate the kexo for exonuclease activity (see Murakami E, Ray A S, Schinazi R F, Anderson K S. Investigating the effects of stereochemistry on incorporation and removal of 5-fluorocytidine analogs by mitochondrial DNA polymerase gamma: comparison of D- and L-D4FC-TP. Antiviral Res. 2004; 62: 57-64; Feng J Y, Murakami E, Zorca S M, Johnson A A, Johnson K A, Schinazi R F, Furman P A, Anderson K S. Relationship between antiviral activity and host toxicity: comparison of the incorporation efficiencies of 2′,3′-dideoxy-5-fluoro-3′-thiacytidine-triphosphate analogs by human immunodeficiency virus type 1 reverse transcriptase and human mitochondrial DNA polymerase. Antimicrob Agents Chemother. 2004; 48: 1300-6).

### Example 9

### Inhibition of Human DNA Polymerases by NTPs

### Study Objectives

To determine whether a nucleoside-triphosphate analog inhibits human DNA polymerases Alpha, Beta and Gamma and to calculate IC₅₀ values.

### Materials and Methods

Human DNA Polymerase Alpha—Enzyme can be purchased from Chimerx (cat #1075) and assayed based on their recommendations with some modifications. The 2′-Me-UTP was treated with Inorganic Pyrophosphatase (Sigma) to remove any pyrophosphate contamination. A final concentration of 500 μM 2′-Me-UTP can be incubated with 1 mM DTT, 50 mM Tris, 50 mM NaCl, 6 mM MgCl₂, and 1 unit of pyrophosphatase for 1 hour at 37° C. followed by inactivation at 95° C. for 10 minutes. A mixture of 0.05 units of Human DNA Polymerase Alpha and a 5′end radiolabeled 24nt DNA primer (5′-TCAGGTCCCTGTTCGGGCGCCACT) anneal to a 48nt DNA template (5′-CAGTGTGGAAAATCTCTAGCAGTGGCGCCCGAACAGGGACCTGAAAGC) can be mixed with increasing concentrations of compound from 0 to 100 μM in 60 mM Tris-HCl (pH 8.0), 5 mM magnesium acetate, 0.3 mg/ml bovine serum albumin, 1 mM dithiothreitol, 0.1 mM spermine, 0.05 mM of each dCTP, dGTP, dTTP, dATP in a final reaction volume of 20 μl for 5 min at 37° C. (all concentrations represent final concentrations after mixing). The reactions can be stopped by mixing with 0.3 M (final) EDTA. Products are separated on a 20% polyacrylamide gel and quantitated on a Bio-Rad Molecular Imager FX. Results from the experiments can be fit to a dose response equation, (y min+((y max)−(y min)))/(1+(compound concentration)/IC₅₀)\hat{ }slope) to determine IC₅₀ values using Graphpad Prism or SynergySoftware Kaleidagraph. Data can be normalized to controls.

Human DNA Polymerase Beta—Enzyme can be purchased from Chimerx (cat #1077) and assayed based on their recommendations with some modifications. A mixture of 0.1 units of Human DNA Polymerase Beta and a 5′end radiolabeled 24nt DNA primer (5′-TCAGGTCCCTGTTCGGGCGCCACT) anneal to a 48nt DNA template (5′-CAGTGTGGAAAATCTCTAGCAGTGGCGCCCGAACAGGGACCTGAAAGC) can be mixed with increasing concentrations of compound from 0 to 100 μM in 50 mM Tris-HCl (pH 8.7), 10 mM KCl, 10 mM MgCl₂, 0.4 mg/ml bovine serum albumin, 1 mM dithiothreitol, 15% (v/v) glycerol, and 0.05 mM of each dCTP, dGTP, dTTP, dATP in a final reaction volume of 20 μl for 5 min at 37° C. (all concentrations represent final concentrations after mixing). The reactions can be stopped by mixing with 0.3 M (final) EDTA. Products can be separated on a 20% polyacrylamide gel and quantitated on a Bio-Rad Molecular Imager FX. Results from the experiments can be fit to a dose response equation, (y min+((y max)−(y min)))/(1+(compound concentration)/IC₅₀)\hat{ }slope) to determine IC₅₀ values using Graphpad Prism or SynergySoftware Kaleidagraph. Data can be normalized to controls.

Human DNA Polymerase Gamma—Enzyme can be purchased from Chimerx (cat #1076) and assayed based on their recommendations with some modifications. A mixture of 0.625 units of Human DNA Polymerase Gamma and a 5′end radiolabeled 24nt DNA primer (5′-TCAGGTCCCTGTTCGGGCGCCACT) anneal to a 36nt DNA template (5′-TCTCTAGAAGTGGCGCCCGAACAGGGACCTGAAAGC) can be mixed with increasing concentrations of compound from 0 to 100 μM in 50 mM Tris-HCl (pH 7.8), 100 mM NaCl, 5 mM MgCl₂, and 0.05 mM of each dCTP, dGTP, dTTP, dATP in a final reaction volume of 20 μl for 200 min at 37° C. (all concentrations represent final concentrations after mixing). The reactions can be stopped by mixing with 0.3 M (final) EDTA. Products can be separated on a 20% polyacrylamide gel and quantitated on a Bio-Rad Molecular Imager FX. Results from the experiments can be fit to a dose response equation, (y min+((y max)−(y min)))/(1+(compound concentration)/IC₅₀))\hat{ }slope) to determine IC₅₀ values using Graphpad Prism or SynergySoftware Kaleidograph. Data can be normalized to controls.

### Example 10

### Cellular Pharmacology in HepG2 Cells

HepG2 cells are obtained from the American Type Culture Collection (Rockville, MD), and are grown in 225 cm² tissue culture flasks in minimal essential medium supplemented with non-essential amino acids, 1% penicillin-streptomycin. The medium is renewed every three days, and the cells are subcultured once a week. After detachment of the adherent monolayer with a 10 minute exposure to 30 mL of trypsin-EDTA and three consecutive washes with medium, confluent HepG2 cells are seeded at a density of 2.5×10⁶ cells per well in a 6-well plate and exposed to 10 μM of [³H] labeled active compound (500 dpm/pmol) for the specified time periods.

The cells are maintained at 37° C. under a 5% CO₂ atmosphere. At the selected time points, the cells are washed three times with ice-cold phosphate-buffered saline (PBS).

Intracellular active compound and its respective metabolites are extracted by incubating the cell pellet overnight at −20° C. with 60% methanol followed by extraction with an additional 20 pal of cold methanol for one hour in an ice bath. The extracts are then combined, dried under gentle filtered air flow and stored at −20° C. until HPLC analysis.

### Example 101

### Cellular Pharmacology in PBM Cells

Test compounds are incubated in PBM cells at 50 μM for 4 h at 37° C. Then the drug containing media is removed and the PBM cells are washed twice with PBS to remove extracellular drugs. The intracellular drugs are extracted from 10×10⁶ PBM cells using 1 mL 70% ice-cold methanol (containing 10 nM of the internal standard ddATP). Following precipitation, the samples are maintained at room temperature for 15 min followed by vortexing for 30 sec, and then stored 12 h at −20° C. The supernatant is then evaporated to dryness. Dry samples would be stored at −20° C. until LC-MS/MS analysis. Prior to analysis, each sample is reconstituted in 100 μL mobile phase A, and centrifuged at 20,000 g to remove insoluble particulates.

Gradient separation is performed on a Hypersil GOLD column (100×1.0 mm, 3 μm particle size; Thermo Scientific, Waltham, MA, USA). Mobile phase A consists of 2 mM ammonium phosphate and 3 mM hexylamine. Acetonitrile is increased from 10 to 80% in 15 min, and kept at 80% for 3 min. Equilibration at 10% acetonitrile lasts 15 min.

The total run time is 33 min. The flow rate is maintained at 50 μL/min and a 10 μL injection is used. The autosampler and the column compartment are typically maintained at 4.5 and 30° C., respectively.

The first 3.5 min of the analysis is diverted to waste. The mass spectrometer is operated in positive ionization mode with a spray voltage of 3.2 kV.

### Example 12

### Anti-Enterovirus Activity

Compounds are tested for cytotoxicity using a MTS (3-(4,5-dimethylthiazol-2-yl)-5-(3-carboxymethoxyphenyl)-2-(4-sulfophenyl)-2H-tetrazolium) method and the CC₅₀ (IC₅₀) values (cytotoxic concentrations of drug required to reduce cell viability by 50%) are determined for each test compound in human rhabdomyosarcoma (RD) cell line. In addition, the maximum non-toxic concentrations (MNTC) are also determined for each compound. To avoid potential drug toxicity that would interfere with viral cytopathic effect (CPE), compounds are evaluated at nontoxic concentrations. The resultant inhibitory effect of each test compound is calculated as a percentage of decrease in EV-71 CPE. Briefly, a monolayer of RD cells is prepared in a 96-well plates. The cells are then infected with 1 MOI of EV-71 (BrCr strain) followed by treatment with a single non-toxic dose of each compound in triplicate. The vehicle control wells are treated with 0.1% DMSO diluted in the working media. The plate is then incubated for 48 h at which time the virus-control wells produced detectable CPE. To determine the probable CPE inhibitory effect of test compounds, an MTS assay is performed and effective compounds are chosen for further studies to identify the potency of the compounds and their concentration-dependent manner. The dose-response antiviral activity of each compound is determined by a virus yield reduction assay method. Briefly, confluent monolayers of RD cells in 96-wells microplate are infected with 0.1 MOI of EV-71 followed by treatment with compounds. Effective compounds are further quantified and confirmed with a virus-yield-reduction assay using an optimized in house qRT-PCR to determine the EV-71 RNA copy number after 2 days post-treatment from collected supernatants. qRT-PCR is performed using the EV-71 specific probe/primer mix and qScript-Tough master mix (Quantibio, USA). Quantitative PCR measurement was performed using StepOnePlus real time PCR system (Roche, Germany) according to manufacturer's protocol. The median effective concentration (EC₅₀) and the concentration with 90% of inhibitory effect (EC₉₀) are calculated using GraphPad PRISM for Windows, version 5 (GraphPad Software Inc., San Diego, C A, 2005) as the means±standard deviation (SD) of the mean from triplicate assay from three independent experiments.

### Example 13

### Chikungynya Virus Antiviral Activity Assay

Methods for evaluating the efficacy of the compounds described herein against Chikungunya virus, a representative Togaviridae virus, is shown, for example, in Ehteshami, M., Tao, S., Zandi, K., Hsiao, H. M., Jiang, Y., Hammond, E., Amblard, F., Russell, O.O., Mertis, A., and Schinazi, R. F.: Characterization of β-D-N4-hydroxycytidine as a novel inhibitor of chikungunya virus. Antimirob Agents Chemother, 2017 April; 61(4): e02395-16.

Anti-Chikungunya Activity can also be evaluated as outlined in “Anti-Chikungunya Viral Activities of Aplysiatoxin-Related Compounds from the Marine Cyanobacterium Trichodesmium erythraeum” Gupta, D. K.; Kaur, P.; Leong, S. T.; Tan, L. T.; Prinsep, M. R.; Chu, J J. H. Mar Drugs. January 2014; 12(1): 115-127; 10.3390/md12010115 and references cited therein.

### Example 14

### Assaying Compounds for Efficacy Against Mavaro Virus Infection:

A representative assay for determining the efficacy of the compounds described herein against the Mayaro virus, another representative Togaviridae virus, is disclosed in Cavalheiro et al., “Macrophages as target cells for Mayaro virus infection: involvement of reactive oxygen species in the inflammatory response during virus replication,” Anais da Academia Brasileira de Ciências (2016) 88(3): 1485-1499, (Annals of the Brazilian Academy of Sciences). The procedures are summarized below.

### Cell Culture and Virus Propagation

RAW 264.7, a mouse leukaemic macrophage cell line, and J774, a mouse reticulum sarcoma cell line, can be maintained in RPMI-1640 medium (LGC) supplemented with 10% fetal bovine serum (FBS; Invitrogen Life Technologies) in a humidified incubator at 37° C. with 5% C02. Mouse peritoneal macrophages can be obtained from C57Bl/6 animals by the intraperitoneal injection of 1 mL of sterile 3% thioglycollate. After 96 h, the peritoneal macrophages can be harvested, washed with RPMI and centrifuged at 1,500 rpm for five minutes. Then, the macrophages can be plated at a density of 2×10⁶ cells/well in a 6-well plate with RPMI-1640 supplemented with 10% FBS and incubated at 37° C. with 5% C02. After 24 h, the plates can be washed with RPMI to remove non-adherent cells before the assays.

MAYV (ATCC VR 66, strain TR 4675) and SINV (AR339) can be propagated in BHK-21 cells grown in α-Minimum Essential Medium (α-MEM; Invitrogen Life Technologies) supplemented with 10% FBS. The cells can be infected with a multiplicity of infection (MOI) of 0.1. After 16 h for SINV and 30 h for MAYV, the culture media can be harvested and cell debris can be removed by centrifugation at 2,000×g for 10 min and the supernatant can be stored at −80° C. Virus stocks titers can be determined by plaque assay in BHK-21 cells.

### Macrophage Infection Assays

Cells can be incubated with MAYV or SINV at a MOI of 1 (for RAW 264.7 and J774) or 5 (for primary peritoneal macrophages), for 1 h at 37° C. in 5% C02. Then, the medium containing the non-adsorbed virus can be removed, the cells can be washed with serum-free medium and cultured in RPMI supplemented with 5% FBS, at 37° C. in 5% C02. After the desired periods of infection, conditioned media can be collected for virus titration, LDH assay and cytokine quantification. Cellular extracts can be used for MTT and flow cytometry assays. Virus inactivated by heating at 65° C. for 30 min can be used as control. In some experiments, cells can be treated with 10 mM N-acetyl-L-cysteine (NAC; Sigma-Aldrich) or 50 μM apocynin (Sigma-Aldrich) for 15 h after infection with MAYV.

### Virus Titration by Plaque Assay

BHK-21 cells can be seeded, for example, at a density of 1.25×10⁵ cells per well in 12-wells plates and incubated at 37° C. overnight. Ten-fold serial dilutions of the virus samples can be prepared in α-MEM and incubated with the cells for 1 h at 37° C. (0.2 mL per well). After 1 h adsorption, 2 mL of 1% carboxymethylcellulose (w/v) (Sigma-Aldrich) in α-MEM supplemented with 2% FBS can be layered onto the infected monolayers and the cells can be incubated at 37° C. for 30 h or 48 h, for SINV or MAYV, respectively. Plaques can be visualized by staining the monolayer with 1 mL 1% crystal violet in 20% ethanol.

### Cell Viability Assays

Determination of macrophage viability during infection can be assessed by 3-(4,5-dimethylthiazol-2-yl)-2,5-diphenyltetrazolium bromide (MTT) or lactate dehydrogenase (LDH) release assays. For the MTT assay, cells can be incubated with 0.5 mL 0.5 mg/mL MTT (USB Corporation) in PBS solution for 90 min at 37° C. Then, unreacted dye can be discarded and formazan crystals can be An Acad Bras Cienc (2016) 88 (3) 1488 Mariana G. Cavalheiro et al. solubilized in 0.04 M HCl solution in isopropanol (1 mL per well). The absorbance of samples can be measured at 570 nm and 650 nm for background correction. Lactate dehydrogenase (LDH) release from infected macrophages can be determined by using an LDH detection kit (Promega CytoTox 96 assay kit). The procedures can be performed according to manufacturer's instructions.

### Quantitation of Infected Cells by Flow Cytometry

Flow cytometry analysis can be performed to assess the frequency of MAYV- or SINV-infected cells by detecting intracellular viral antigens. After the desired periods of infection, cells can be washed with PBS, detached by scraping, harvested and fixed in 4% formaldehyde in PBS at room temperature for 15 min. After washing, cells can be permeabilized with 0.1% saponin in PBS and incubated with blocking solution (PBS supplemented with 2% FBS and 0.1% bovine serum albumin) for 20 min, at room temperature. Then, cells can be incubated for 1 h with mouse anti-Eastern Equine Encephalitis virus monoclonal antibody (Chemicon International, Millipore), which reacts with an E1 epitope shared by all alphaviruses. Then, cells can be washed and stained with anti-mouse IgG conjugated to Alexa Fluor 488 (Invitrogen) for 30 min. The percentage of infected cells can be analyzed by FACScan Flow Cytometer and CellQuest software (Becton Dickinson).

### Characterization of Cell Death

Apoptosis/necrosis after infection can be quantified by a double staining method using The Vybrant Apoptosis Assay Kit #2 (Molecular Probes). After the infection period, RAW 264.7 cells can be washed with PBS, detached by scraping, harvested and stained with Annexin V Alexa Fluor 488 (0.5 μg/mL) and propidium iodide (PI, 0.25 μg/mL). To further characterize MAYV-induced cell death, the activity of caspases 3 and 7 can be measured using the Muse™ Caspase-3/7 Kit (Millipore) adapted to flow cytometry. Cells can be washed with PBS, detached by scraping, harvested and incubated with Muse™ Caspase-3/7 Reagent 1.8 and Muse™ Caspase 7-AAD, according to the manufacturer's protocol. For both assays, the percentage of apoptotic and necrotic cells can be analyzed by FACScan Flow Cytometer using the CellQuest software (Bectan Dickinson). UV radiated cells and cells subjected to a freeze-thaw procedure can be used as controls.

### Quantitation of Reactive Oxygen Species (ROS)

The amount of intracellular reactive oxygen species (ROS) can be measured by the formation of the oxidized derivative of 5-(and 6-)-chloromethyl-2′,7′-dichlorodihydrofluorescein diacetate (DCF, Molecular Probes). After 15 h of infection with MAYV, adherent cells can be washed with PBS and incubated with DCF 0.5 μM for 45 minutes. Then, the cells can be washed again, detached by scraping and harvested and analyzed by FACScan Flow Cytometer using the CellQuest software (Bectan Dickinson).

Quantitation of Cytokines The concentrations of cytokines in the conditioned medium of macrophage cultures can be determined by ELISA. TNF concentration can be quantified using the Standard ELISA Development kit (PeproTech), according to the manufacturer's protocol.

### Example 15

### Yellow Fever Virus (YFV) Antiviral Activity Assay: Primary Assay for Antiviral Activity

A monolayer of human Rhabdomyosarcoma (RD) cells will be grown in 96-well plate in MEM containing 2% inactivated FBS. The tested compounds will be added to the wells in triplicate together with YFV at an MOI=1. The plate will then be incubated at 37° C. with 5% C02 for 72 hours. The assay will be conducted in triplicate for each concentration of each compound. After three days, the plate will be viewed under the microscope and the degree of cytopathic effect (CPE) as measure of virus replication inhibition will be expressed as the percent yield of virus control. The results will be evaluated by performing the MTS assay (Promega, WI, USA) according to the manufacturer's protocols. All experiments will be repeated three times independently.

### Focus Forming Unit Reduction Assay (FFURA)

Antiviral activity of each compound will be determined by measuring the reduction in the number of YFV infectious foci in RD cells following treatment with increasing concentrations of each compound. Briefly, infected RD cells which will be treated with different concentrations of each compound will be incubated for 2 days post infection using conditioned-growth medium supplemented with 2% FBS and 1.5% carboxymethyl cellulose (CMC). Antiviral activity of each compound will be determined after visualizing and counting viral foci. The number of YFV foci will be counted using Elispot machine and the virus titer will be expressed as Foci Forming-Unit (FFU). Antiviral activities of the compounds will be determined by calculating the percentage of foci reduction (% RF) against the controls maintained in parallel using the following formula; RF (%)=(C-T)×100/C, where, C is the mean of the number of foci from triplicates treatment without compound added (vehicle control) and T is the mean of the number of foci from triplicates of each treatment measures with the respective compound. Results will represent as the means±standard error of the mean (SEM) from triplicate assay from three independent experiments. Results were confirmed by virus yield reduction assay using quantitative RT-PCR.

### Virus Yield Reduction Assay

Monolayers of RD cells will be prepared in 96-well cell culture microplate and overlaid with YFV (moi=0.1) for 1 hour. After virus adsorption, cells will be washed 3 times with cold sterile PBS to remove unattached viruses and then the cells will be treated for 2 days with increasing concentrations of the tested compounds. After 2 days, the YFV RNA will be extracted from the infected/treated cells and supernatant separately and the yield of YFV will quantified using a one-step specific quantitative RT-PCR for YFV. Nevertheless, the antiviral activity of each nucleoside analogues will be investigated using focus forming unit reduction assay (FFURA) as described previously

### Time-of-Drug-Addition Assay

Confluent monolayers of RD cells will be prepared in 96-well cell culture plate and will be pre-treated with EC₉₀ of each effective drug for 2 h before infection with YFV at MOI=1, concurrently with infection as well as 1, 2, 4 and 6 h post-infection. The cells will then be incubated in the presence of compound for 48 h. At the end of the incubation period, viral load for each time point of treatment will be determined using qRT-PCR as mentioned above.

### Statistical Analysis

Graph Pad Prism for Windows, Version 5 (Graph Pad Software Inc., San Diego, C A, 2005) will be used to determine the half maximal effective concentration EC₅₀ values and also EC₉₀ of each effective compound. All EC₅₀ and EC₉₀ values will be calculated as the means standard error of the mean (SEM) from triplicate assay from three independent experiments.

### Example 16

### Efficacy of the Compounds Described Herein Against Dengue

The essential role of a particular viral protein (Dengue virus envelope protein (E)) in viral propogation. Mondotte et al., J. Virol. July 2007, vol. 81 no. 13 7136-7148 discloses an assay useful for identifying compounds for treating infections caused by the Dengue virus, and this assay can be used to identify those compounds described herein which are active against Dengue.

Another assay is described in Levin, 14th International Symposium on Hepatitis C Virus & Related Viruses, Glasgow, UK, 9-13 Sep. 2007. The assay relates to human and Dengue virus polymerase, where putative compounds can be tested against the enzymes, preferably in duplicate, over a range of concentrations, such as from 0.8 mM to 100 mM. The compounds can also be run alongside a control (no inhibitor), a solvent dilution (0.016% to 2% DMSO) and a reference inhibitor.

A suitable high throughput assay for Dengue is described in Lim et al., Antiviral Research, Volume 80, Issue 3, December 2008, Pages 360-369. Dengue virus (DENV) NS5 possesses methyltransferase (MTase) activity at its N-terminal amino acid sequence and is responsible for formation of a type 1 cap structure, m7GpppAm2′-0 in the viral genomic RNA. Optimal in vitro conditions for DENV2 2′-O-MTase activity can be characterized using purified recombinant protein and a short biotinylated GTP-capped RNA template. Steady-state kinetics parameters derived from initial velocities can be used to establish a robust scintillation proximity assay for compound testing. Pre-incubation studies by Lim et al., Antiviral Research, Volume 80, Issue 3, December 2008, Pages 360-369, showed that MTase-AdoMet and MTase-RNA complexes can be equally catalytically competent and the enzyme supports a random bi kinetic mechanism. Lim validated the assay with competitive inhibitory agents, S-adenosyl-homocysteine and two homologues, sinefungin and dehydrosinefungin. A GTP-binding pocket present at the N-terminal of DENV2 MTase can be previously postulated to be the cap-binding site. This assay allows rapid and highly sensitive detection of 2′-O-MTase activity, and can be readily adapted for high-throughput screening for inhibitory compounds.

### Example 17

### Anti-Norovirus Activity

Compounds can exhibit anti-norovirus activity by inhibiting norovirus polymerase and/or helicase, by inhibiting other enzymes needed in the replication cycle, or by other pathways.

There is currently no approved pharmaceutical treatment for Norovirus infection (http://www.cdc.gov/ncidod/dvrd/revb/gastro/norovirus-qa.htm), and this has probably at least in part been due to the lack of availability of a cell culture system. Recently, a replicon system has been developed for the original Norwalk G-I strain (Chang, K. O., et al. (2006) Virology 353:463-473).

Both Norovirus replicons and hepatitis C replicons require viral helicase, protease, and polymerase to be functional in order for replication of the replicon to occur. Most recently, an in vitro cell culture infectivity assay has been reported utilizing Norovirus genogroup I and II inoculums (Straub, T. M. et al. (2007) Emerg. Infect. Dis. 13(3):396-403). This assay is performed in a rotating-wall bioreactor utilizing small intestinal epithelial cells on microcarrier beads. The infectivity assay may be useful for screening entry inhibitors.

### Diagnosis of Norovirus Infection

One can diagnose a norovirus infection by detecting viral RNA in the stools of affected persons, using reverse transcription-polymerase chain reaction (RT-PCR) assays. The virus can be identified from stool specimens taken within 48 to 72 hours after onset of symptoms, although one can obtain satisfactory results using RT-PCR on samples taken as long as 7 days after the onset of symptoms. Other diagnostic methods include electron microscopy and serologic assays for a rise in titer in paired sera collected at least three weeks apart. There are also commercial enzyme-linked immunoassays available, but these tend to have relatively low sensitivity, limiting their use to diagnosis of the etiology of outbreaks. Clinical diagnosis of norovirus infection is often used, particularly when other causative agents of gastroenteritis have been ruled out.

### Example 18

### Determining the Efficacy of the Compounds Against ZIKV and DENV Infection

### Material and Methods for ZIKV and DENV (Serotypes 1-4) Infections Assays:

Viruses: ZIKV PRVABC59 strain (NCBI accession KU501215) was obtained from the Centers for Diseases Control and Prevention. Virus stocks were generated on C6/36 or Vero cells and viral titers are determined by endpoint titration in Vero (African Green monkey kidney) or human cells, including neuroblastoma (U251), and hepatoblastoma (Huh7). DENV stocks (kindly provided by Dr. Guey Chuen Perng (Emory University & National Cheng Kung University, Taiwan) were generated in Vero or Baby Hamster Kidney cells (BHK) (Clark et al., 2016).

Cytopathic-reduction assay for ZIKV or DENV: For the cytopathic-reduction assay, cells (Vero, U251 or Huh7) are seeded in 96-well plates at 1×10⁴ cells/well and incubated overnight. The next day, culture medium containing 50% cell culture infectious doses of ZIKV or DENV (tested in Vero or BHK cells) are added after which 2-fold serial dilutions of the compounds are added. Cell cytopathic effect (CPE) is measured by MTS readout system (CellTiter 96 AQueous One Solution Proliferation kit, Promega) four (Vero) or five (U251 or Huh7) days after compound addition to determine the levels of ZIKV replication inhibition (Zmurko et al., 2016; Gavegnano et al., 2017). For DENV serotypes 1-4, CPE is measured four to five days after compound addition in Vero or BHK cells.

Focus formation assay: For the focus formation assay (FFA), Vero cells are routinely seeded in 96-well plates at 1.5×10⁴ cells/well and incubated overnight. Next, culture medium containing 70-100 focus forming units of ZIKV or DENV (serotypes 1-4) plus 2-fold serial dilutions of the compounds are added to the cells and incubated for 2 h followed by the addition of overlay methylcellulose medium. Following 2-3 days of incubation, foci are stained using anti-Flavivirus group antigen (4G2, Millipore), followed by HRP-anti-mouse IgG and TrueBlue substrate, and imaged using CTL-Immunospot S6 Micro Analyzer (Priyamvada et al., 2016).

Real-time RT-PCR assay: For the RT-PCR assays, Vero, U251, or Huh7 cells (15,000/well) are seeded in 96-well microplates, and cultured overnight prior to use for infections with ZIKV (MOI=0.001 for Vero or MOI-0.5 for U251 or Huh7) or DENV (with MOI varying from 0.001 to 0.1 for different stocks of serotypes 1-4 for Vero cells). Compounds are added at a dose-dependent manner 1-2 h after ZIKV or DENV. After four days incubation, purified RNA are reverse transcribed into cDNA and amplified in a one-step RT-PCR multiplex reaction with LightCycler 480 RNA Master Hydrolysis Probe (Roche, Indianapolis, IN) using highly conserved sequences complementary to a 76 bp fragment from the ZIKV envelope gene as previously described by Lanciotti (Lanciotti et al., 2008), and an endogenous control (TaqMan Ribosomal RNA Control or beta globin reagents; Applied Biosystems) by using the LightCycler 480 Instrument II (Roche). For detection of dengue viruses, we utilized oligonucleotides primers and probes serotype-specific that rapidly detects all four serotypes in a fourplex RT-PCR assay (Johnoson et al., 2005). For all virological tests, percent inhibition and EC₅₀ value (compound concentration that inhibits viral antigen expression or viral replication by 50%) are determined using CalcuSyn software (Biosoft).

Combination studies for ZIKV or DENV.

Ideally, active compounds for use in treating ZIKV or DENV have sub-μM concentrations for hit to lead development, with cell selectivity index (SI)≥100. Hit compounds that demonstrate antiviral potency with no apparent cytotoxicity can be selected for drug-drug combinations with compounds that exhibit different mechanism of action, including viral entry and host inhibitors, among others; These combinations can result in synergistic effects and optimal low doses to rapidly eliminate ZIKV or DENV from infected individuals.

One can use the Chou and Talalay method (Chou & Talalay 1984) for determining synergy, antagonism or additivity (Bassit et al., 2008; Schinazi et al., 2012), particularly with respect to combinations.

### Material and Methods for DENV2 (Serotype 2) Replicon Assay:

Baby hamster kidney (BHK-21) stable cell lines expressing dengue virus serotype 2 [DENV2, New Guinea C strain, Qing et al., 2010)] was kindly provided by Mehul S. Suthar (Emory University).

DENV2 replicon-harboring baby hamster kidney (BHK) cells are exposed to test compounds at concentrations varying from 0.2 to 20 μM to assessment of antiviral activity. Renilla luciferase levels (Promega) are quantified 48 hours after test compounds addition to determine the levels of replication inhibition (EC₅₀, M).

### REFERENCES

1. Clark, K. B., Hsiao, H. M., Bassit L., Crowe J. E. Jr., Schinazi R. F., Perng G. C., Villinger F. Characterization of dengue virus 2 growth in megakaryocyte-erythrocyte progenitor cells. Virology. 493, 162-72 (2016).
2. Zmurko, J., Marques, R. E., Schols, D., Verbeken, E., Kaptein, S. J. F. & Neyts, J. The Viral Polymerase Inhibitor 7-Deaza-2′-C-Methyladenosine Is a Potent Inhibitor of In Vitro Zika Virus Replication and Delays Disease Progression in a Robust Mouse Infection Model. PLoS Neglected Tropical Diseases 10, e0004695, doi:10.1371/journal.pntd.0004695 (2016).
3. Gavegnano C, Bassit L C, Cox B D, Hsiao H-M, Johnson E L, Suthar M, Chakraborty R, Schinazi R F. Jak inhibitors modulate production of replication-competent Zika Virus in Human Hofbauer, Trophoblasts, and Neuroblastoma cells. Pathogens & immunity. 2, 199-218 (2017).
4. Priyamvada L, Quicke K M, Hudson W H, Onlamoon N, Sewatanon J, Edupuganti S, Pattanapanyasat K, Chokephaibulkit K, Mulligan M J, Wilson P C, Ahmed R, Suthar M S, Wrammert J. Human antibody responses after dengue virus infection are highly cross-reactive to Zika virus. PNAS 113, 7852-7857, (2016).
5. Lanciotti R, Kosoy O, Laven J, Velez J, Lambert A, Johnson A, Stanfield S, Duffy M. Genetic and serologic properties of Zika virus associated with an epidemic, Yap State, Micronesia, 2007. Emerg Infect Dis. 14, 1232-1239 (2008).
6. Johnson B W, Russell B J, Lanciotti R S. Serotype-specific detection of dengue viruses in a fourplex real-time reverse transcriptase PCR assay. J Clin Microbiol 43(10), 4977-4983 (2005).
7. Chou T C, Talalay P. Quantitative analysis of dose-effect relationships: the combined effects of multiple drugs or enzyme inhibitors. Adv Enzyme Regul. 22, 27-55 (1984).
8. Bassit L, Grier J, Bennett M, Schinazi R F. Combinations of 2′-C-methylcytidine analogues with interferon-alpha2b and triple combination with ribavirin in the hepatitis C virus replicon system. Antivir Chem Chemother. 19(1), 25-31 (2008).
9. Schinazi R F, Bassit L, Clayton M M, Sun B, Kohler J J, Obikhod A, Arzumanyan A, Feitelson M A. Evaluation of single and combination therapies with tenofovir disoproxil fumarate and emtricitabine in vitro and in a robust mouse model supporting high levels of hepatitis B virus replication. Antimicrob Agents Chemother. 56(12), 6186-91 (2012).
10. Qing M, Liu W, Yuan Z et al., A high-throughput assay using dengue-1 virus like particles for drug discovery. Antiviral Res. 86(2), 163-71 (2010).

### Example 19

### MERS Assay

### Cells and Virus.

Human lung carcinoma cells (A-549) can be used for the primary antiviral assays and can be obtained from American Type Culture Collection (ATCC, Rockville, Md., USA). The cells can be passed in minimal essential medium (MEM with 0.15% NaCHO₃, Hyclone Laboratories, Logan, Utah, USA) supplemented with 10% fetal bovine serum. When evaluating compounds for efficacy, the serum can be reduced to a final concentration of 2% and the medium can contain gentamicin (Sigma-Aldrich, St. Louis, Mo.) at 50 μg/mL. Since the MERS-Co virus did not produce detectable virus cytopathic effects, virus replication in A549 cells can be detected by titering virus supernatant fluids from infected, compound-treated A549 cells in Vero 76 cells.

Vero 76 cells can be obtained from ATCC and can be routinely passed in MEM with 0.15% NaCHO₃ supplemented with 5% fetal bovine serum. When evaluating compounds, the serum can be reduced to a final concentration of 2% and supplemented with 50 μg/mL of gentamicin.

The Middle Eastern coronavirus strain EMC (MERS-CoV) was an original isolate from humans that was amplified in cell culture by Ron Fouchier (Erasmus Medical Center, Rotterdam, the Netherlands) and was obtained from the Centers for Disease Control (Atlanta, Ga.).

### Controls:

Infergen® (interferon alfacon-1, a recombinant non-naturally occurring type-I interferon (Blatt, L., et al., J. Interferon Cytokine Res. (1996) 16(7):489-499 and Alberti, A., BioDrugs (1999) 12(5):343-357) can be used as the positive control drug in all antiviral assays. Infergen=0.03 ng/mL.

### Antiviral Assay:

Virus can be diluted in MEM to a multiplicity of infection=0.001 and each compound can be diluted in MEM+2% FBS using a half-log 8 dilution series. Compound can be added first to 96 well plates of confluent A549 cells followed within 5 mins by virus. Each test compound dilution can be evaluated for inhibition in triplicate. After plating, the plates can be incubated at 37° C. for 4 d. The plates can then be frozen at −80° C.

### Virus Yield Reduction Assay:

Infectious virus yields from each well from the antiviral assay can be determined. Each plate from an antiviral assays can be thawed. Samples wells at each compound concentration tested can be pooled and titered for infectious virus by CPE assay in Vero 76 cells. The wells can be scored for CPE and virus titers calculated. A 90% reduction in virus yield can then be calculated by regression analysis. This represented a one log₁0 inhibition in titer when compared to untreated virus controls.

### Example 20

### Venezuelan Equine Encephalitis Virus (VEEV) Assay

96-well plates of HeLa-Ohio cells can be prepared and incubated overnight. The plates can be seeded at 4×10⁴ cells per well, which yields 90-100% confluent monolayers in each well after overnight incubation. The test compounds in DMSO can be started at a concentration of 100 μM. 8-fold serial dilutions in MEM medium with 0.1% DMSO, 0% FBS, and 50 μg/mL gentamicin with the test compound concentrations being prepared. To 5 test wells on the 96-well plate can be added 100 μL of each concentration and the plate can be incubated at 37° C. +5% CO₂ for 2 h or 18 h. 3 wells of each dilution with the TC-83 strain Venezuelan equine encephalitis virus (ATCC, stock titer: 10⁶0.8 CCID₅₀/mL) prepared in the medium as described above can be added. 2 wells (uninfected toxicity controls) can be added MEM with no virus. 6 wells can be infected with untreated virus controls. To 6 wells can be added media only as cell controls. A blind, known active compound can be tested in parallel as a positive control. The plate can be incubated at 37° C. +5% C02 for 3 d. The plate can be read microscopically for visual CPE and a Neutral red dye plate can also be read using BIO-TEK Instruments INC. EL800. For virus yield reduction assays, the supernatant fluid can be collected from each concentration. The temperature can be held at −80° and each compound can be tested in triplicate. The CC₅₀ can be determined by regression analysis using the CPE of toxicity control wells compared with cell controls. The virus titers can be tested in triplicate using a standard endpoint dilution CCID₅₀ assay and titer calculations can be determined using the Reed-Muench (1948) equation. The concentration of compound required to reduce virus yield by 1 log₁₀ (90%) using regression analysis can be calculated (EC₉₀ value). The concentration of compound required to reduce virus yield by 50% using regression analysis can be calculated (EC₅₀ value).

### Example 21

### Rift Valley Fever Assay

The compounds described herein can be tested for activity against Rift Valley Fever virus using methods known to those skilled in the art (e.g., described in Panchal et al., Antiviral Res. (2012) 93(1):23-29).

### Example 22

### Determining the Efficacy of the Compounds Against HCoV-OC43 and SARS-CoV-2 Infections

### Viruses

HCoV-OC43 was obtained from ATCC (Manasas, VA) and SARS-CoV-2 was provided by BEI Resources (NR-52281. USA-WA/2020). HCoV-OC43 and SARS-CoV-2 were propagated in Huh-7 and Vero cells, respectively and titrated by TCID₅₀ method followed by storage of aliquots at −80° C. until further use.

Antiviral Activity Assay using Virus Yield Assay Method

To determine the best time point for the virus yield assay, a kinetic replication of SARS-CoV-2 and HCoV-OC43 in Vero, Caco2, Calu3 and Huh-7 cells was performed, respectively, and the yield of progeny virus was assessed from the supernatant of viral infected cells at different interval time points using specific q-RT PCR for each virus as mentioned earlier. We determined that 48 and 72 h post-infection were the optimum time point for SARS-CoV-2 and HCoV-OC43, respectively, as there was no observed cell death and cytopathic effect (CPE) on infected cells and more importantly, significant increase in the virus RNA copy number which harvested from the supernatant of the infected cells were observed at that time for SARS-CoV-2 and also HCoV-OC43.

In the next step towards defining the antiviral activity of each compound, we have assessed the antiviral activity of each compound in a dose-dependent manner against SARS-CoV-2 and HCoV-OC43 using a virus yield inhibition assay by determining the viral RNA copy number in collected supernatants, compared to the results from infected but untreated cells, and non-infected and untreated cells as necessary controls. All experiments were performed in triplicate and each experiment repeated three times independently to achieve reliable and statistically meaningful results.

The median effective concentration (EC₅₀) and the concentration with 90% of inhibitory effect (EC₉₀) were calculated using GraphPad PRISM for Mac, version 7 (GraphPad Software Inc., San Diego, C A, 2005) and reported as the mean±standard deviation (SD).

### Example 23

### Enzymatic Evaluation of SARS-CoV-2 RNA Dependent RNA Polymerase Inhibitors

The severe acute respiratory syndrome-coronavirus 2 (SARS-CoV-2) outbreak has caused a global coronavirus (COVID-19) pandemic. The RNA-dependent RNA polymerase (RdRp), also known as nsp12, is a core component of the virus replication and transcription complex and handles the replication and transcription of viral RNA (Yi Jiang, et al., “RNA-dependent RNA polymerase: Structure, mechanism, and drug discovery for COVID-19,” Biochemical and Biophysical Research Communications, Volume 538, 2021, Pages 47-53). RdRp also appears to be a primary target for the antiviral drug remdesivir.

COVID-19 virus nsp12 forms a complex with cofactors nsp7 and nsp8. The nsp12-nsp7-nsp8 complex includes the domain organization of COVID-19 virus nsp12. The interdomain borders are labeled with residue numbers. This complex is disclosed in Gao et al., “Structure of the RNA-dependent RNA polymerase from COVID-19 virus,” Science 368 (6492), 779-782 (2020).

An inhibitor triphosphate can interfere with RNA synthesis. An RNA polymerase is an enzyme that synthesizes RNA from a DNA template. When a growing RNA chain comes into contact with an RNA polymerase and a naturally-occurring nucleoside triphosphate, the RNA chain is extended. However, when an unnatural inhibitor triphosphate is present, there is an error when the RNA polymerase seeks to add the inhibitor triphosphate to the growing RNA chain.

To measure the ability of modified nucleoside triphosphate inhibitors to disrupt RNA synthesis, a 0.1 μM RdRP complex (reconstituted from three individual nsp proteins) can be prepared by mixing the three proteins, then incubating them on ice for 30 minutes.

The complex can be buffered using 25 mM Tris-HCl (pH 8). To this buffered solution of the RdRP complex can be added 50 μM of a 17-mer RNA primer and 1 μM of a 43-mer RNA template, and the solution can be incubated on ice for around 15 minutes.

Once the complex is formed, 0.1 μM hot GTP can be added, followed by addition of NTP mixtures (50 mM ATP, CTP and TTP; 25 mM GTP) to provide the nucleoside triphosphates needed for RNA synthesis. Then, either control (water) or an inhibitor triphosphate can be added.

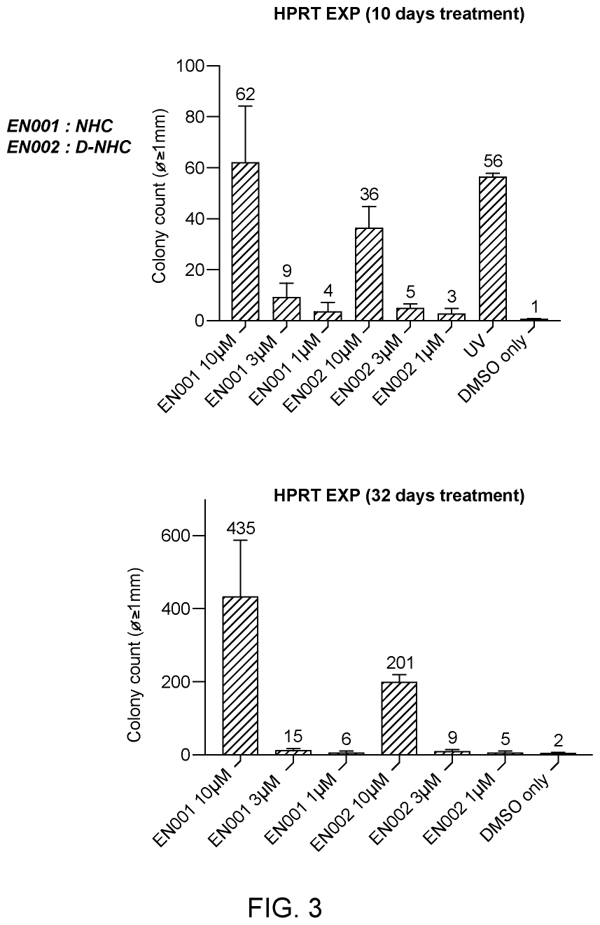

The results are shown in FIG. 3 and in the following table. FIG. 3 is a chart showing the results of a mammalian cell HPRT gene mutation assay: CHO-K1 cells (hamster cell line) were treated with drugs at the concentration of 10, 3 and 1 μM. After 6-thioguanine selection, resistant cell colonies were counted as a measure of HPRT knockout mutations in host cells. D-NHC reduces the number of colony formed by about 50% compared with NHC. As shown in the table and figure,

### Anti-SARS-CoV-2 in Vero Cells and Calu3 Cells and Toxicity Profile

| | Anti-SARS- | | Anti-SARS- | | | | | |
|---|---|---|---|---|---|---|---|---|
| | CoV-2 | | CoV-2 | | | | | |
| | Activity | | Activity | | Cytotoxicity | | | |
| | (Vero) μM | | (Calu3) μM | | CC₅₀ (μM) | | | |
| Compound/Structure | EC₅₀ | EC₉₀ | EC₅₀ | EC₉₀ | PBM | CEM | Vero | Huh7 |
| 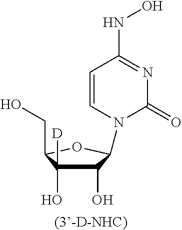 | 0.1 | 0.25 | 0.2 | 0.9 | 24.1 | | 41.6 | 27.7 |
| 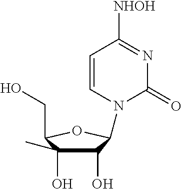 | 0.6 | 7.4 | >100 | >100 | >100 | >100 | >100 | ≥100 |
| 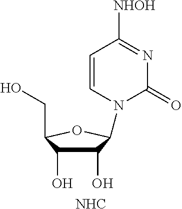 | 0.2 | 0.42 | 0.6 | 2.0 | 49.2 | 2.6 | 16.5 | 80.3 |

The present invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

Various publications are cited herein, the disclosures of which are incorporated by reference in their entireties.

1. A compound of Formula (A) 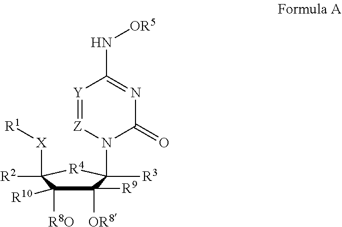
or a pharmaceutically acceptable salt or prodrug thereof, wherein:
X is CH₂, —CH(CH₃)—, CH(CH₃)₂—. CHF, CF₂ or CD₂,
Y is N or CR′,
Z is N or CR″,
R′ is H, deuterium or fluorine,
R″ is H, deuterium or methyl,
R¹ is OH, an optionally substituted O-linked amino acid, —O—C(O)—C₁₋₁₂ alkyl, —O—C(O)—C₂₋₁₂ alkenyl, —O—C(O)—C₂₋₁₂ alkynyl, —O—C(O)—C₃₋₆ cycloalkyl, —O—C(O)O—C₁₋₁₂ alkyl, —O—C(O)O—C₂₋₁₂ alkenyl, —O—C(O)O—C₂₋₁₂ alkynyl, —O—C(O)O—C₃₋₆ cycloalkyl, OC₁₋₆ alkyl, OC₁₋₆ haloalkyl, OC₁₋₆ alkoxy, OC₂₋₆ alkenyl, OC₂₋₆ alkynyl, OC₃₋₆ cycloalkyl, O—P(O)R⁶R⁷, O—CH₂—P—(OH)₃, O—CH₂—P—(OH)₃, or a mono-, di-, or triphosphate, wherein, when chirality exists at the phosphorous center of R⁴, it may be wholly or partially Rₚ or Sₚ or any mixture thereof,
R⁶ and R⁷ are independently selected from the group consisting of:
(a) OR¹⁵ where R¹⁵ selected from the group consisting of H, 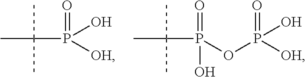
Li, Na, K, substituted or unsubstituted C₁₋₂₀alkyl, substituted or unsubstituted C₃₋₆cycloalkyl, C₁₋₄ (alkyl)aryl, benzyl, C₁₋₆haloalkyl, C₂₋₃(alkyl)OC₁₋₂₀alkyl, aryl, and heteroaryl, such as phenyl and pyridinyl, wherein aryl and heteroaryl are optionally substituted with zero to three substituents independently selected from the group consisting of (CH₂)₀₋₆CO₂R¹⁶ and (CH₂)₀₋₆ CON(R¹⁶)₂;
where R¹⁶ is independently H, substituted or unsubstituted C₁₋₂₀ alkyl, the carbon chain derived from a fatty alcohol or C₁₋₂₀ alkyl substituted with a C₁₋₆ alkyl, C₁₋₆ alkoxy, di(C₁₋₆ alkyl)-amino, fluoro, C₃₋₁₀ cycloalkyl, cycloalkyl-C₁₋₆ alkyl, cycloheteroalkyl, aryl, heteroaryl, substituted aryl, or substituted heteroaryl; wherein the substituents are C₁₋₅ alkyl, or C₁₋₅ alkyl substituted with a C₁₋₆alkyl, alkoxy, di(C₁₋₆ alkyl)-amino, fluoro, C₃₋₁₀ cycloalkyl, or cycloalkyl;
(b) the ester of a D- or L-amino acid 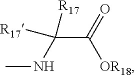
R¹⁷, R^{17′} and R¹⁸ are independently H, C₁₋₂₀ alkyl, the carbon chain derived from a fatty alcohol or C₁₋₂₀ alkyl optionally substituted with a C₁₋₆ alkyl, alkoxy, di(C₁₋₆alkyl)-amino, fluoro, C₃₋₁₀ cycloalkyl, cycloalkyl-C₁₋₆ alkyl, cycloheteroalkyl, aryl, heteroaryl, substituted aryl, or substituted heteroaryl; wherein the substituents are C₁₋₅ alkyl, or C₁₋₅ alkyl substituted with a C₁₋₆alkyl, alkoxy, di(C₁₋₆alkyl)-amino, fluoro, C₃₋₁₀ cycloalkyl, or cycloalkyl;
R² is H, deuterium, F, CN, N³, C₁₋₃ alkyl, C₂₋₃ alkynyl,
R³ is H, deuterium, CN, N³, C₁₋₃ alkyl, C₂₋₃ alkynyl,
R⁴ is O, CH₂, S, Se, CHF, CF₂, —C(CH₃)—, —C(cyclopropyl)-, C═CF₂ or C═CH₂,
R⁵ is H, an optionally substituted O-linked amino acid, —C(O)—C₁₋₁₂ alkyl, —C(O)—C₂₋₁₂ alkenyl, —C(O)—C₂₋₁₂ alkynyl, —C(O)—C₃₋₆ cycloalkyl, —C(O)O—C₁₋₁₂ alkyl, —C(O)O—C₂₋₁₂ alkenyl, —C(O)O—C₂₋₁₂ alkynyl, —C(O)O—C₃₋₆ cycloalkyl, wherein the groups can be substituted with one or more fluorine,
R⁸ and R^{8′} are independently selected from the group consisting of H, an optionally substituted O-linked amino acid, —C(O)—C₁₋₁₂ alkyl, —C(O)—C₂₋₁₂ alkenyl, —C(O)—C₂₋₁₂ alkynyl, —C(O)—C₃₋₆ cycloalkyl, —C(O)O—C₁₋₁₂ alkyl, —C(O)O—C₂₋₁₂ alkenyl, —C(O)O—C₂₋₁₂ alkynyl, —C(O)O—C₃₋₆ cycloalkyl, wherein the groups can be substituted with one or more substituents selected from the group consisting of fluorine, hydroxyl, amino, alkylamino, arylamino, and alkoxy,
R⁹ is H or deuterium, and
R¹⁰ is deuterium or methyl
or a compound of Formula (B) 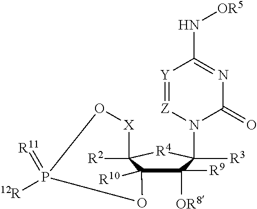
Formula B
or a pharmaceutically acceptable salt or prodrug thereof, wherein:
X is CH₂, —CH(CH₃)—, CH(CH₃)₂—. CHF, CF₂ or CD₂,
Y is N or CR′,
Z is N or CR″,
R′ is H, deuterium or fluorine,
R″ is H, deuterium or methyl,
R² is H, deuterium, F, CN, N³, C₁₋₃ alkyl, or C₂₋₃ alkynyl,
R³ is H, deuterium, CN, N³, C₁₋₃ alkyl, or C₂₋₃ alkynyl,
R⁴ is O, CH₂, S, Se, CHF, CF₂, —C(CH₃)—, —C(cyclopropyl)-, C═CF₂ or C═CH₂,
R⁵ is H, an optionally substituted O-linked amino acid, —C(O)—C₁₋₁₂ alkyl, —C(O)—C₂₋₁₂ alkenyl, —C(O)—C₂₋₁₂ alkynyl, —C(O)—C₃₋₆ cycloalkyl, —C(O)O—C₁₋₁₂ alkyl, —C(O)O—C₂₋₁₂ alkenyl, —C(O)O—C₂₋₁₂ alkynyl, —C(O)O—C₃₋₆ cycloalkyl, wherein the groups can be substituted with one or more fluorine atoms,
R^{8′} is selected from the group consisting of H, an optionally substituted O-linked amino acid, —C(O)—C₁₋₁₂ alkyl, —C(O)—C₂₋₁₂ alkenyl, —C(O)—C₂₋₁₂ alkynyl, —C(O)—C₃₋₆ cycloalkyl, —C(O)O—C₁₋₁₂ alkyl, —C(O)O—C₂₋₁₂ alkenyl, —C(O)O—C₂₋₁₂ alkynyl, —C(O)O—C₃₋₆ cycloalkyl, wherein the groups can be substituted with one or more substituents selected from the group consisting of fluorine, hydroxyl, amino, alkylamino, arylamino, and alkoxy,
R⁹ is H or deuterium,
R¹⁰ is deuterium or methyl,
R¹¹ is O or S, and
R¹² is selected from the group consisting of
(a) OR¹⁵ where R¹⁵ is selected from the group consisting of H, substituted or unsubstituted C₁₋₂₀ alkyl, substituted or unsubstituted C₃₋₆cycloalkyl, C₁₋₄(alkyl)aryl, benzyl, C₁₋₆ haloalkyl, C₂₋₃ (alkyl)OC₁₋₂₀ alkyl, aryl, and heteroaryl, such as phenyl and pyridinyl, wherein aryl and heteroaryl are optionally substituted with zero to three substituents independently selected from the group consisting of (CH₂)₀₋₆CO₂R¹⁶ and (CH₂)₀₋₆ CON(R¹⁶)₂;
(b) the ester of a D- or L-amino acid 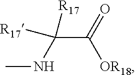
R¹⁷ and R¹⁸ are independently H, C₁₋₂₀ alkyl, the carbon chain derived from a fatty alcohol or C₁₋₂₀ alkyl optionally substituted with a C₁₋₆ alkyl, alkoxy, di(C₁₋₆alkyl)-amino, fluoro, C₃₋₁₀ cycloalkyl, cycloalkyl-C₁₋₆ alkyl, cycloheteroalkyl, aryl, heteroaryl, substituted aryl, or substituted heteroaryl; wherein the substituents are C₁₋₅ alkyl, or C₁₋₅ alkyl substituted with a C₁₋₆alkyl, alkoxy, di(C₁₋₆alkyl)-amino, fluoro, C₃₋₁₀ cycloalkyl, or cycloalkyl; and
(c) 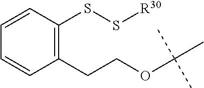
where R³⁰ is selected from the group consisting of substituted or unsubstituted C₁₋₂₀alkyl, substituted or unsubstituted C₃₋₆ cycloalkyl, substituted or unsubstituted (C₂₋₁₀)alkene, substituted or unsubstituted (C₂₋₁₀)alkyne, C₁₋₄(alkyl)aryl, aryl, heteroaryl, and C₁₋₆ haloalkyl,
or a compound of Formula (C) 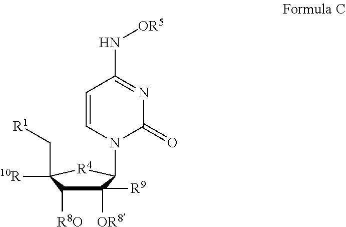
or a pharmaceutically acceptable salt or prodrug thereof, wherein:
R¹ is OH, an optionally substituted O-linked amino acid, —O—C(O)—C₁₋₁₂ alkyl, —O—C(O)—C₂₋₁₂ alkenyl, —O—C(O)—C₂₋₁₂ alkynyl, —O—C(O)—C₃₋₆ cycloalkyl, —O—C(O)O—C₁₋₁₂ alkyl, —O—C(O)O—C₂₋₁₂ alkenyl, —O—C(O)O—C₂₋₁₂ alkynyl, —O—C(O)O—C₃₋₆ cycloalkyl, OC₁₋₆ alkyl, OC₁₋₆ haloalkyl, OC₁₋₆ alkoxy, OC₂₋₆ alkenyl, OC₂₋₆ alkynyl, OC₃₋₆ cycloalkyl, O—P(O)R⁶R⁷, O—CH₂—P—(OH)₃, O—CH₂—P—(OH)₃, or a mono-, di-, or triphosphate, wherein, when chirality exists at the phosphorous center of R⁴, it may be wholly or partially Rₚ or Sₚ or any mixture thereof,
R⁶ and R⁷ are independently selected from the group consisting of:
(a) OR¹⁵ where R¹⁵ selected from the group consisting of H, 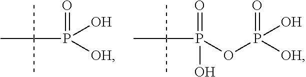
Li, Na, K, substituted or unsubstituted C₁₋₂₀alkyl, substituted or unsubstituted C₃₋₆cycloalkyl, C₁₋₄ (alkyl)aryl, benzyl, C₁₋₆haloalkyl, C₂₋₃(alkyl)OC₁₋₂₀alkyl, aryl, and heteroaryl, such as phenyl and pyridinyl, wherein aryl and heteroaryl are optionally substituted with zero to three substituents independently selected from the group consisting of (CH₂)₀₋₆CO₂R¹⁶ and (CH₂)₀₋₆CON(R¹⁶)₂;
where R¹⁶ is independently H, substituted or unsubstituted C₁₋₂₀ alkyl, the carbon chain derived from a fatty alcohol or C₁₋₂₀ alkyl substituted with a C₁₋₆ alkyl, C₁₋₆ alkoxy, di(C₁₋₆ alkyl)-amino, fluoro C₃₋₁₀ cycloalkyl, cycloalkyl-C₁₋₆ alkyl, cycloheteroalkyl, aryl, heteroaryl, substituted aryl, or substituted heteroaryl; wherein the substituents are C₁₋₅ alkyl, or C₁₋₅ alkyl substituted with a C₁₋₆alkyl, alkoxy, di(C₁₋₆ alkyl)-amino, fluoro, C₃₋₁₀ cycloalkyl, or cycloalkyl;
(b) the ester of a D- or L-amino acid 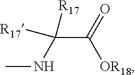
R¹⁷, R^{17′} and R¹⁸ are independently H, C₁₋₂₀ alkyl, the carbon chain derived from a fatty alcohol or C₁₋₂₀ alkyl optionally substituted with a C₁₋₆ alkyl, alkoxy, di(C₁₋₆alkyl)-amino, fluoro, C₃₋₁₀ cycloalkyl, cycloalkyl-C₁₋₆ alkyl, cycloheteroalkyl, aryl, heteroaryl, substituted aryl, or substituted heteroaryl; wherein the substituents are C₁₋₅ alkyl, or C₁₋₅ alkyl substituted with a C₁₋₆alkyl, alkoxy, di(C₁₋₆ alkyl)-amino, fluoro, C₃₋₁₀ cycloalkyl, or cycloalkyl;
R² is H, deuterium, F, CN, N³, C₁₋₃ alkyl, or C₂₋₃ alkynyl,
R⁵ is H, an optionally substituted O-linked amino acid, —C(O)—C₁₋₁₂ alkyl, —C(O)—C₂₋₁₂ alkenyl, —C(O)—C₂₋₁₂ alkynyl, —C(O)—C₃₋₆ cycloalkyl, —C(O)O—C₁₋₁₂ alkyl, —C(O)O—C₂₋₁₂ alkenyl, —C(O)O—C₂₋₁₂ alkynyl, —C(O)O—C₃₋₆ cycloalkyl, wherein the groups can be substituted with one or more fluorine atoms,
R⁸ and R^{8′} are independently selected from the group consisting of H, an optionally substituted O-linked amino acid, —C(O)—C₁₋₁₂ alkyl, —C(O)—C₂₋₁₂ alkenyl, —C(O)—C₂₋₁₂ alkynyl, —C(O)—C₃₋₆ cycloalkyl, —C(O)O—C₁₋₁₂ alkyl, —C(O)O—C₂₋₁₂ alkenyl, —C(O)O—C₂₋₁₂ alkynyl, —C(O)O—C₃₋₆cycloalkyl, wherein the groups can be substituted with one or more substituents selected from the group consisting of fluorine, hydroxyl, amino, alkylamino, arylamino, and alkoxy,
R⁹ is H or deuterium, and
R¹⁰ is deuterium or methyl,
or a compound of Formula (D) 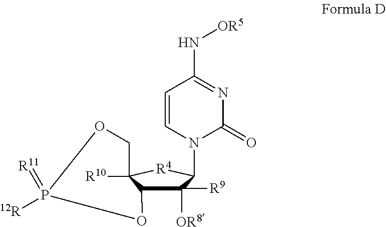
or a pharmaceutically acceptable salt or prodrug thereof, wherein:
R⁵ is H, an optionally substituted O-linked amino acid, —C(O)—C₁₋₁₂ alkyl, —C(O)—C₂₋₁₂ alkenyl, —C(O)—C₂₋₁₂ alkynyl, —C(O)—C₃₋₆ cycloalkyl, —C(O)O—C₁₋₁₂ alkyl, —C(O)O—C₂₋₁₂ alkenyl, —C(O)O—C₂₋₁₂ alkynyl, —C(O)O—C₃₋₆ cycloalkyl, wherein the groups can be substituted with one or more fluorine atoms,
R^{8′} is selected from the group consisting of H, an optionally substituted O-linked amino acid, —C(O)—C₁₋₁₂ alkyl, —C(O)—C₂₋₁₂ alkenyl, —C(O)—C₂₋₁₂ alkynyl, —C(O)—C₃₋₆ cycloalkyl, —C(O)O—C₁₋₁₂ alkyl, —C(O)O—C₂₋₁₂ alkenyl, —C(O)O—C₂₋₁₂ alkynyl, —C(O)O—C₃₋₆ cycloalkyl, wherein the groups can be substituted with one or more substituents selected from the group consisting of fluorine, hydroxyl, amino, alkylamino, arylamino, and alkoxy,
R⁹ is H or deuterium,
R¹⁰ is deuterium or methyl,
R¹¹ is O or S, and
R¹² is selected from the group consisting of
(a) OR¹⁵ where R¹⁵ is selected from the group consisting of H, substituted or unsubstituted C₁₋₂₀alkyl, substituted or unsubstituted C₃₋₆cycloalkyl, C₁₋₄(alkyl)aryl, benzyl, C₁₋₆ haloalkyl, C₂₋₃ (alkyl)OC₁₋₂₀alkyl, aryl, and heteroaryl, such as phenyl and pyridinyl, wherein aryl and heteroaryl are optionally substituted with zero to three substituents independently selected from the group consisting of(CH₂)₀₋₆CO₂R¹⁶ and (CH₂)₀₋₆CON(R¹⁶)₂;
(b) the ester of a D- or L-amino acid 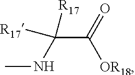
R¹⁷ and R¹⁸ are independently H, C₁₋₂₀ alkyl, the carbon chain derived from a fatty alcohol or C₁₋₂₀ alkyl optionally substituted with a C₁₋₆ alkyl, alkoxy, di(C₁₋₆alkyl)-amino, fluoro, C₃₋₁₀ cycloalkyl, cycloalkyl-C₁₋₆ alkyl, cycloheteroalkyl, aryl, heteroaryl, substituted aryl, or substituted heteroaryl; wherein the substituents are C₁₋₅alkyl or C₁₋₅ alkyl substituted with a C₁₋₆alkyl, alkoxy, di(C₁₋₆alkyl)-amino, fluoro, C₃₋₁₀ cycloalkyl, or cycloalkyl; and
(c) 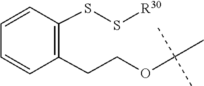
where R³⁰ is selected from the group consisting of substituted or unsubstituted C₁₋₂₀alkyl, substituted or unsubstituted C₃₋₆ cycloalkyl, substituted or unsubstituted (C₂₋₁₀) alkene, substituted or unsubstituted (C₂₋₁₀)alkyne, C₁₋₄(alkyl)aryl, aryl, heteroaryl, and C₁₋₆ haloalkyl,
or a compound of Formula E: 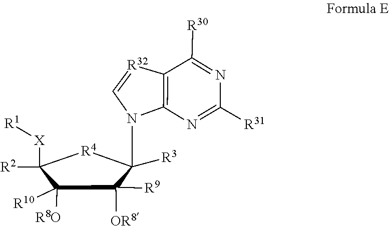
wherein:
R³⁰ is —NH—OH, NH—NH₂, —NH—OMe, —N(Me)-NH₂, or —NH—OR⁵,
R³¹ is H, F, or NH₂,
R³² is CH, CF or N,
and
R¹, R², R³, R⁴, R⁵, R⁶, R⁷, R⁸, R^{8′}, R⁹ and R¹⁰ are as defined in Formulas A and B,
or a compound of Formula F: 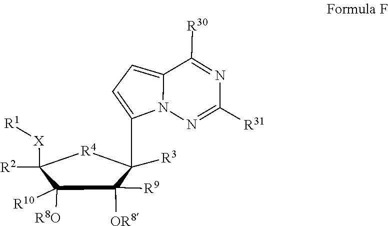
wherein:
R³⁰ is —NH—OH, NH—NH₂, —NH—OMe, —N(Me)-NH₂, or —NH—OR⁵,
R³¹ is H, F, or NH₂, and
R¹, R², R³, R⁴, R⁵, R⁶, R⁷, R⁸, R^{8′}, R⁹ and R¹ are as defined in Formulas A and B,
or a compound of Formula G: 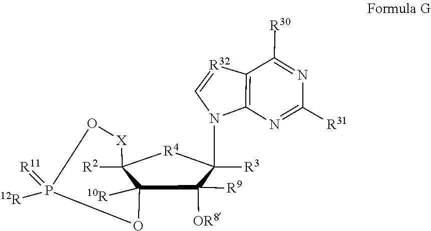
wherein:
R³⁰ is —NH—OH, NH—NH₂, —NH—OMe, —N(Me)-NH₂, or —NH—OR⁵,
R³¹ is H, F, or NH₂,
R³² is CH, CF or N,
and
R², R³, R⁴, R⁵, R^{8′}, R⁹, R¹⁰, R¹¹ and R¹² are as defined in Formulas A and B
or a compound of Formula H: 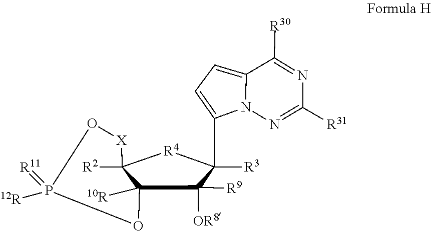
wherein:
R³⁰ is —NH—OH, NH—NH₂, —NH—OMe, —N(Me)-NH₂, or —NH—OR⁵,
R³¹ is H, F, or NH₂, and
R², R³, R⁴, R⁵, R^{8′}, R⁹, R¹⁰, R¹¹ and R¹² are as defined in Formulas A and B.

2. The compound of claim 1, wherein the compound is of Formula A, and:
X is —CH₂—,
one or both of Y and Z are CH,
R¹ is O—P(O)R⁶R⁷, and R⁵ and R⁶ are defined such that R¹ is a phosphoramidate,
R¹ is OH,
R¹ is —O—C(O)—C₁₋₁₂ alkyl,
R² is deuterium,
R³ is CN or N³,
R³ is H,
R⁴ is O,
R⁵ is H,
one or both of R⁸ and R^{8′} are H,
R⁸ is —C(O)—C₁₋₁₂ alkyl,
R¹⁰ is deuterium,
R⁹ is D,
R¹⁰ is methyl, or
one of R⁶ and R⁷ is the ester of a D- or L-amino acid 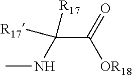
or O-aryl, and the other is O-aryl.

3-18. (canceled)

19. A compound of claim 1, wherein the compound is a compound of Formula B, and:
X is —CH₂—,
one or both of Y and Z are CH,
R² is deuterium,
R³ is CN or N³,
R³ is H,
R⁴ is O,
R⁵ is H,
R^{8′} is H,
R⁹ is D,
R¹⁰ is D,
R¹⁰ is methyl,
R¹¹ is O, or
R¹² is the ester of a D- or L-amino acid

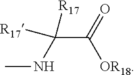

20-32. (canceled)

33. The compound of claim 1, wherein the compound is a compound of Formula C, and:
X is —CH₂—,
one or both of Y and Z are CH,
R¹ is O—P(O)R⁶R⁷, and R⁵ and R⁶ are defined such that R¹ is a phosphoramidate,
R¹ is OH
R¹ is —O—C(O)—C₁₋₁₂ alkyl,
R⁵ is H,
one or R⁶ and R⁷ is the ester of a D- or L-amino acid 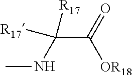
or O-aryl, and the other is O-aryl,
one or both of R⁸ and R^{8′} are H,
R⁸ is —C(O)—C₁₋₁₂ alkyl,
R⁹ is D,
R¹⁰ is deuterium, or
R¹⁰ is methyl.

34-45. (canceled)

46. The compound of claim 1, wherein the compound is a compound of Formula D, and:
R⁴ is O
R⁵ is H,
R^{8′} is H,
R⁹ is D,
R¹⁰ is deuterium,
R¹⁰ is methyl,
R¹¹ is O, or
R¹² is the ester of a D- or L-amino acid

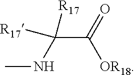

47-54. (canceled)

55. The compound of claim 1, wherein the compound is a compound of Formula D, and:
R¹ is O—P(O)R⁶R⁷, and R⁵ and R⁶ are defined such that R¹ is a phosphoramidate,
R¹ is OH,
R¹ is —O—C(O)—C₁₋₁₂ alkyl,
R² is deuterium,
R³ is CN or N³,
R³ is H,
R⁴ is O,
R⁵ is H
one or R⁶ and R⁷ is the ester of a D- or L-amino acid 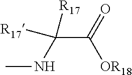
or O-aryl, and the other is O-aryl,
one or both of R⁸ and R^{8′} are H,
R⁸ is —C(O)—C₁₋₁₂ alkyl,
R⁹ is D,
R¹⁰ is deuterium, or
R¹⁰ is methyl.

56-69. (canceled)

70. The compound of claim 69, wherein the compound is a compound of Formula F, and:
R¹ is O—P(O)R⁶R⁷, where R⁵ and R⁶ are defined such that R¹ is a phosphoramidate,
R¹ is OH,
R¹ is —O—C(O)—C₁₋₁₂ alkyl,
R² is deuterium,
R³ is CN or N³,
R³ is H,
R⁴ is O,
R⁵ is H,
one or R⁶ and R⁷ is the ester of a D- or L-amino acid 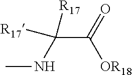
or O-aryl, and the other is O-aryl,
one or both of R⁸ and R^{8′} are H,
R⁸ is —C(O)—C₁₋₁₂ alkyl,
R⁹ is D,
R¹⁰ is deuterium, or
R¹⁰ is methyl.

71-84. (canceled)

85. The compound of claim 1, wherein the compound is a compound of Formula G, and:
R² is deuterium,
R³ is CN or N³,
R³ is H,
R⁴ is O,
R⁵ is H,
R^{8′} is H,
R⁹ is deuterium,
R¹⁰ is deuterium,
R¹⁰ is methyl,
R¹¹ is O, or
R¹² is the ester of a D- or L-amino acid

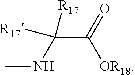

86-96. (canceled)

97. The compound of claim 96, wherein the compound is a compound of Formula H, and:
R² is deuterium,
R³ is CN or N³,
R³ is H,
R⁴ is O,
R⁵ is H,
R^{8′} is H,
R⁹ is deuterium,
R¹⁰ is deuterium,
R¹⁰ is methyl,
R¹¹ is O, or
R¹² is the ester of a D- or L-amino acid

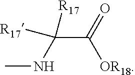

98-107. (canceled)

108. A compound of claim 1, having one of the following formulas: 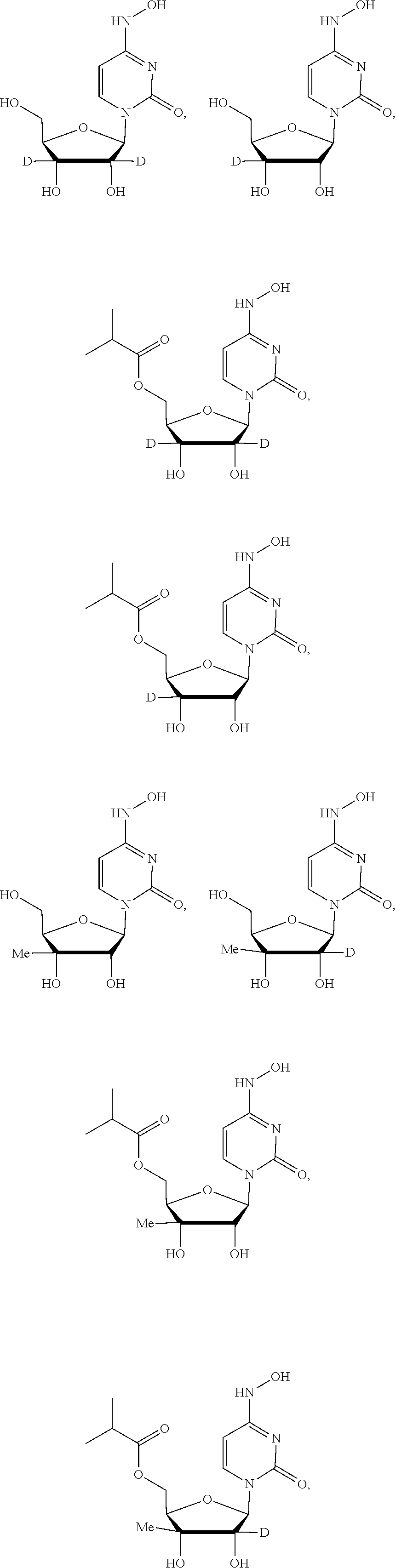
or a pharmaceutically-acceptable salt or prodrug thereof.

109. A compound of claim 1, having one of the following formulas: 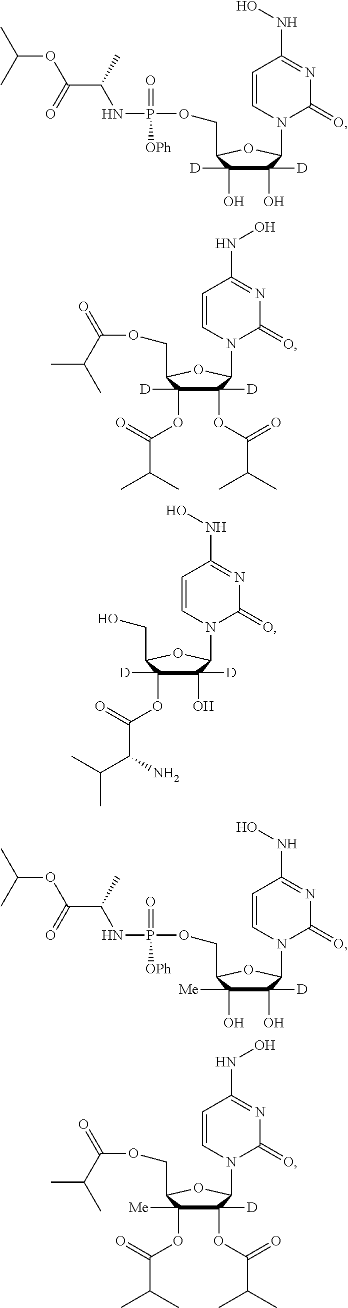 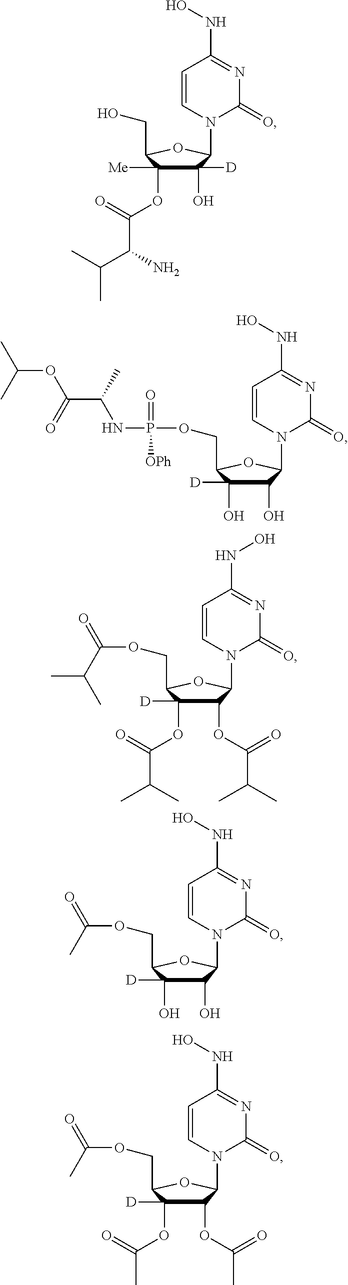 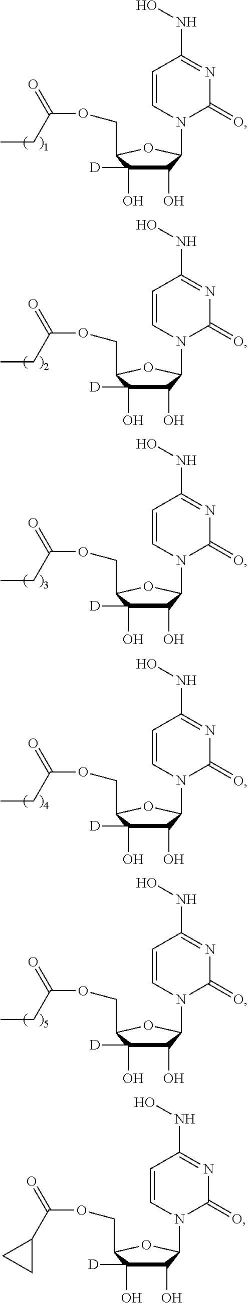 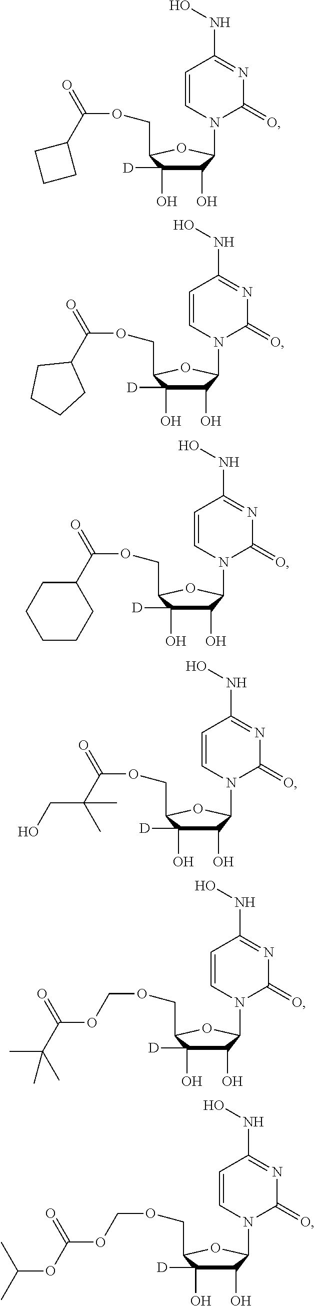 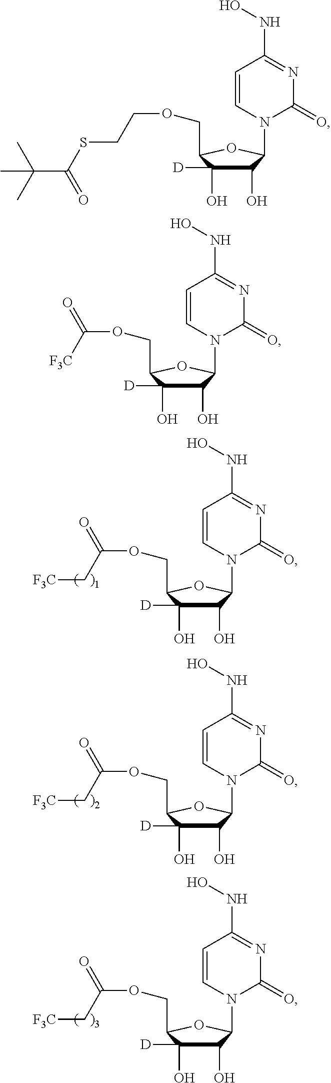 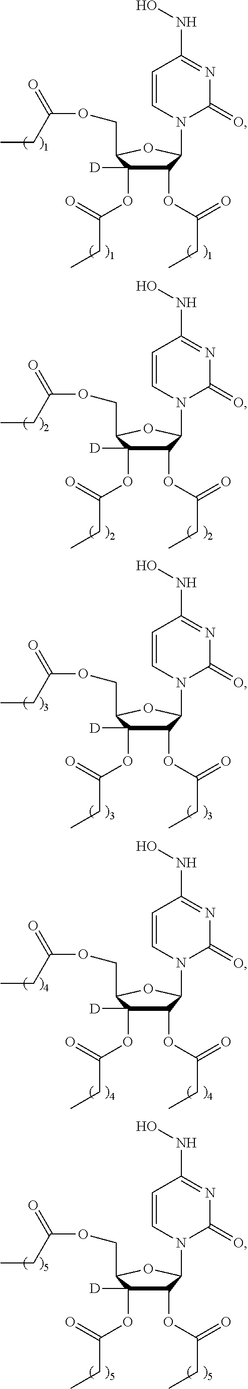 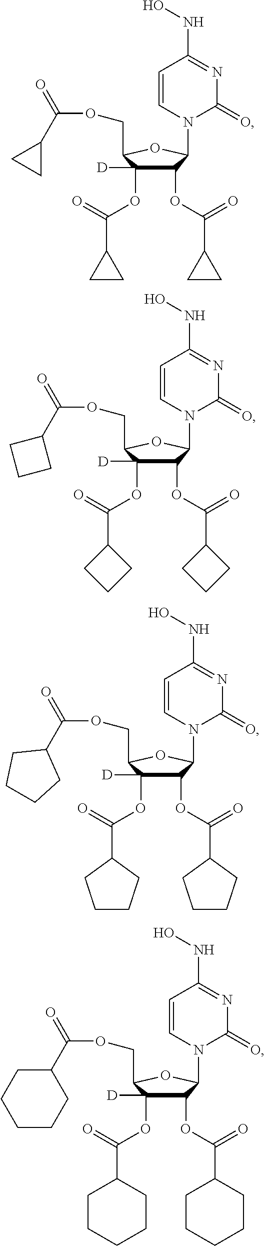 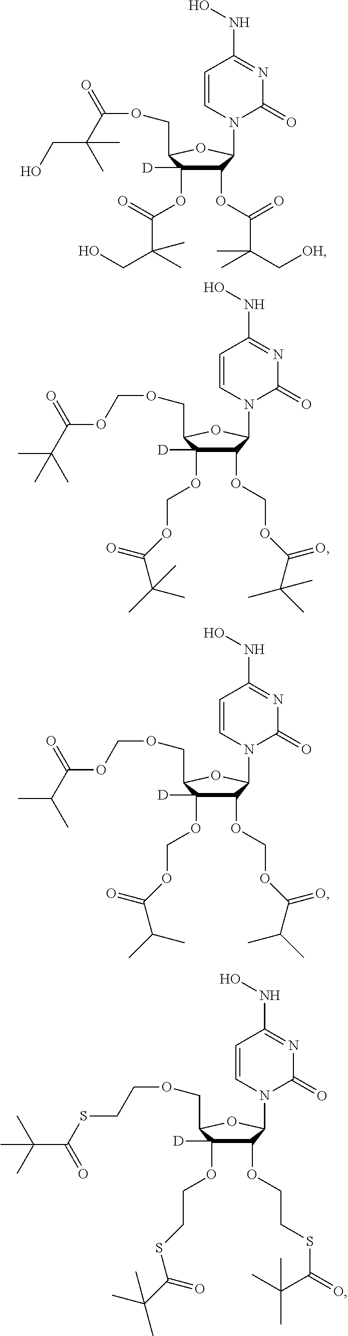 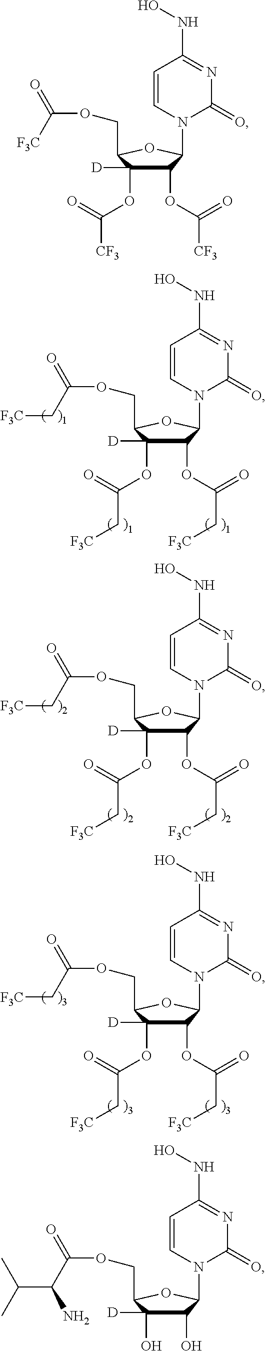 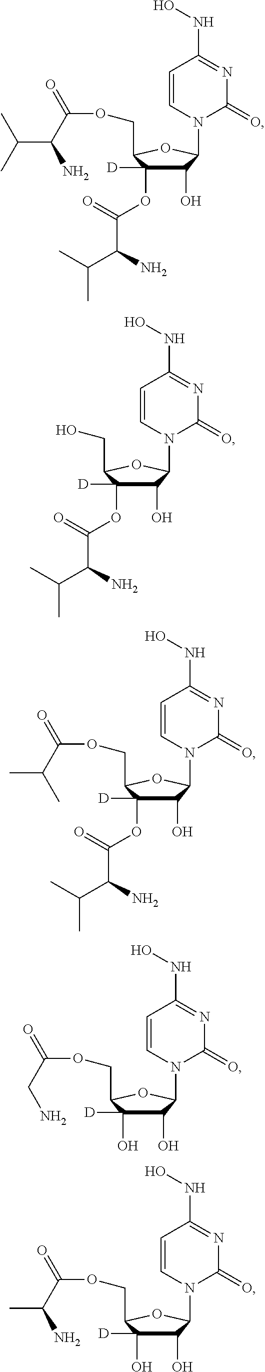 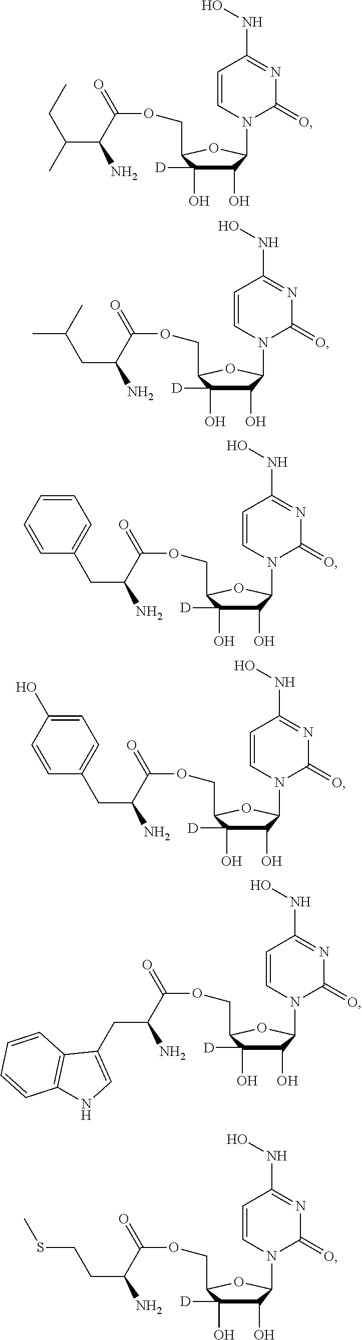 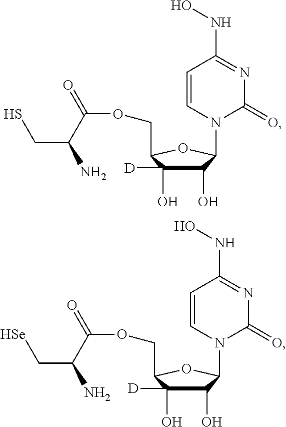 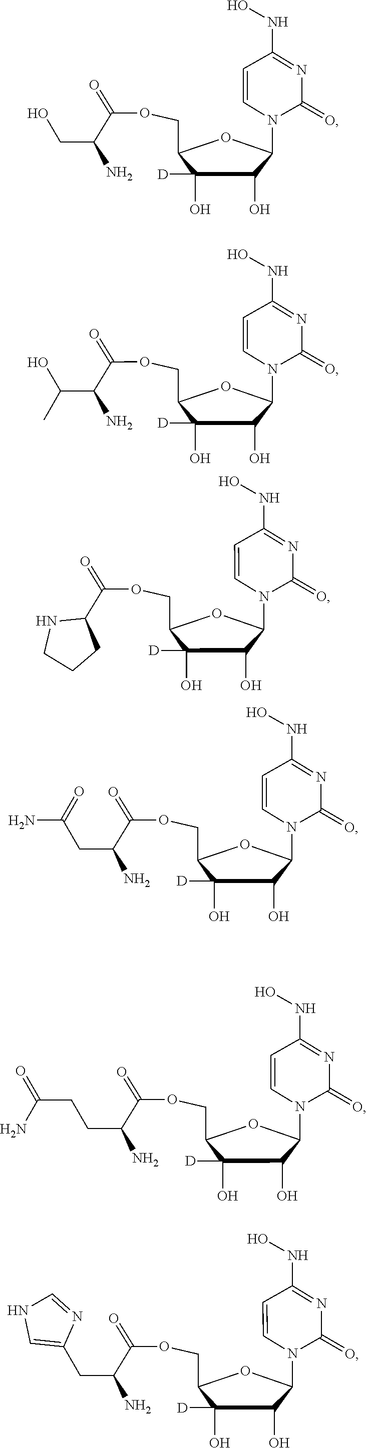 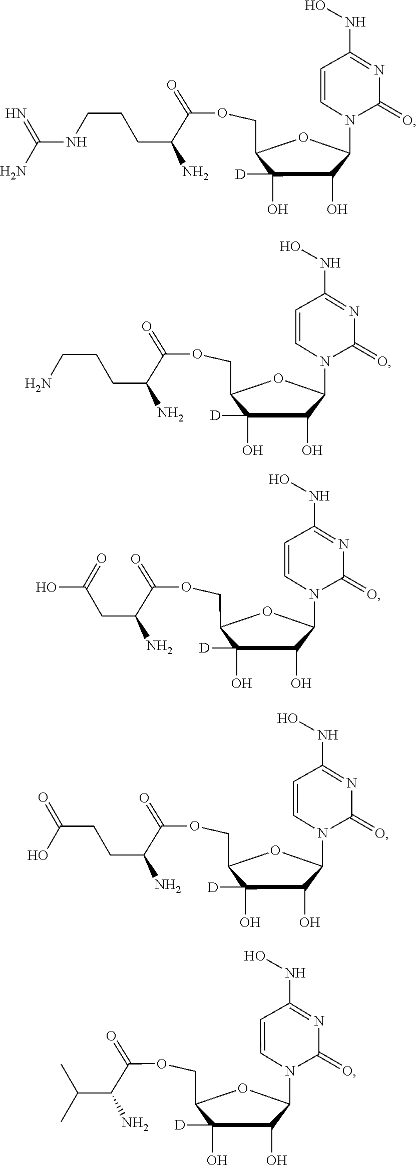 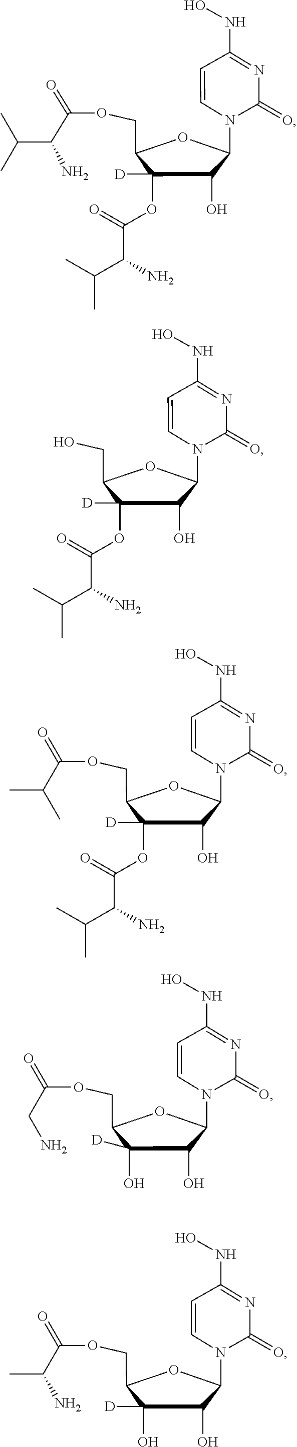 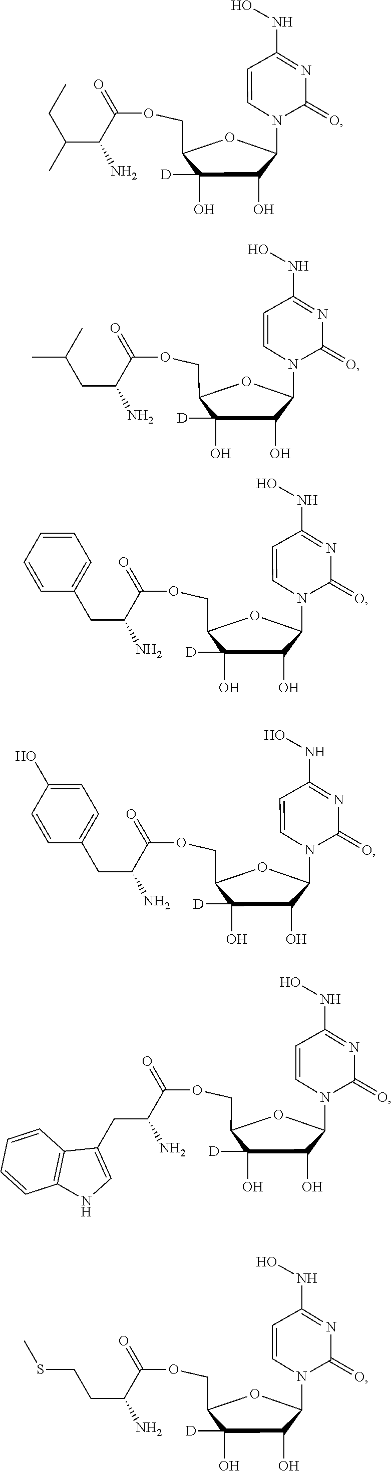 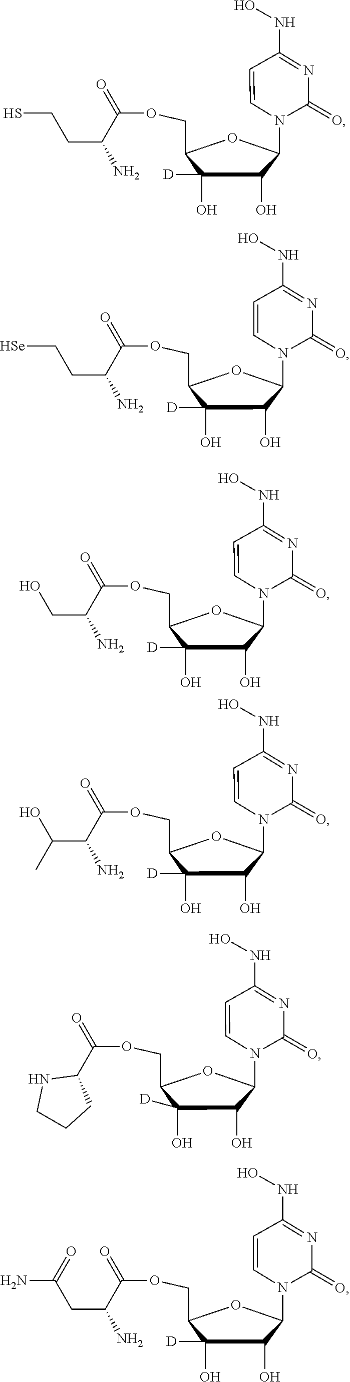 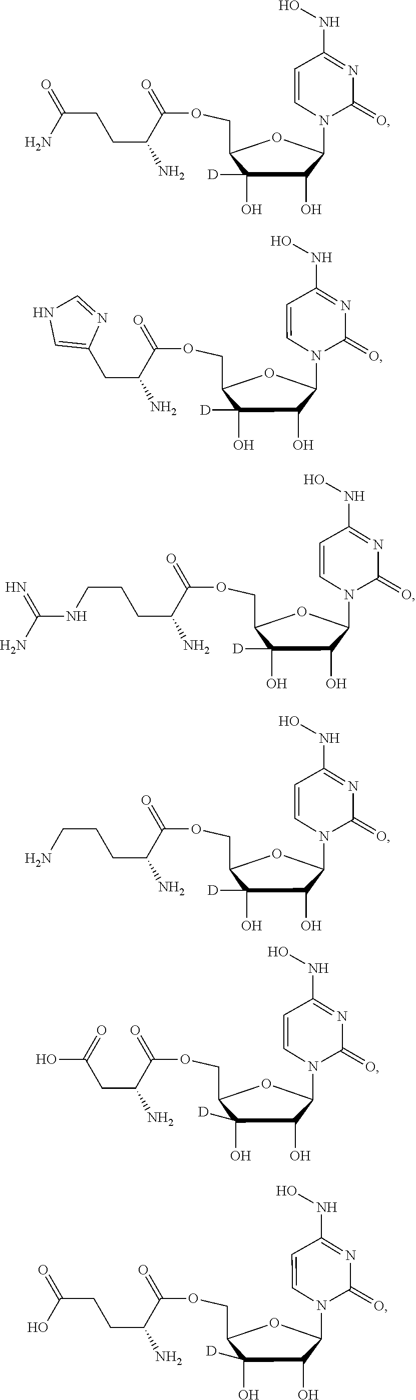 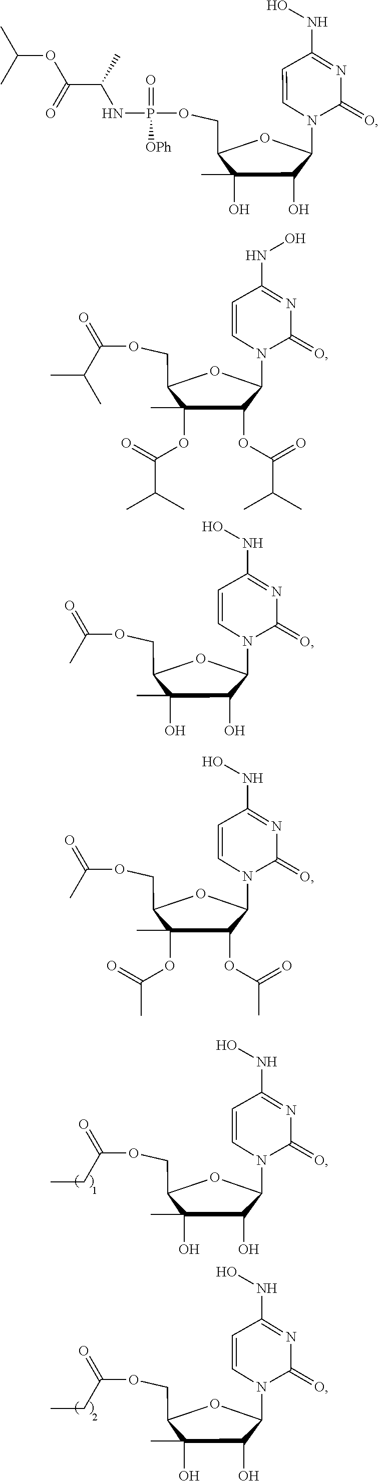 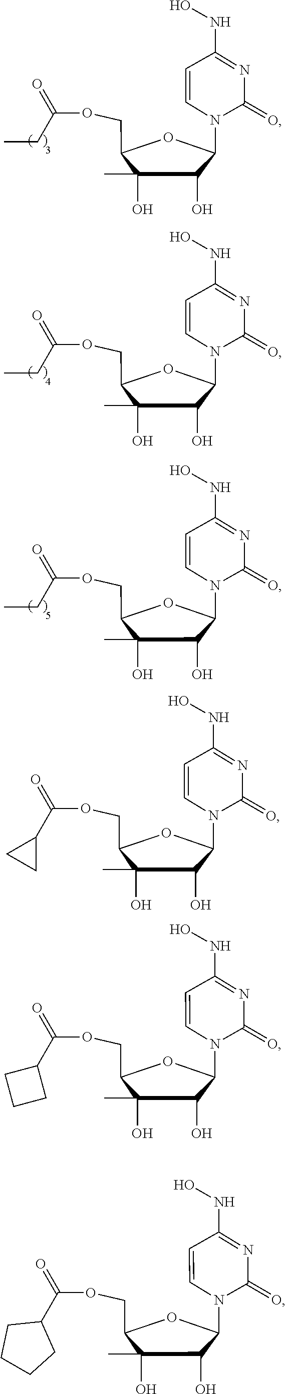 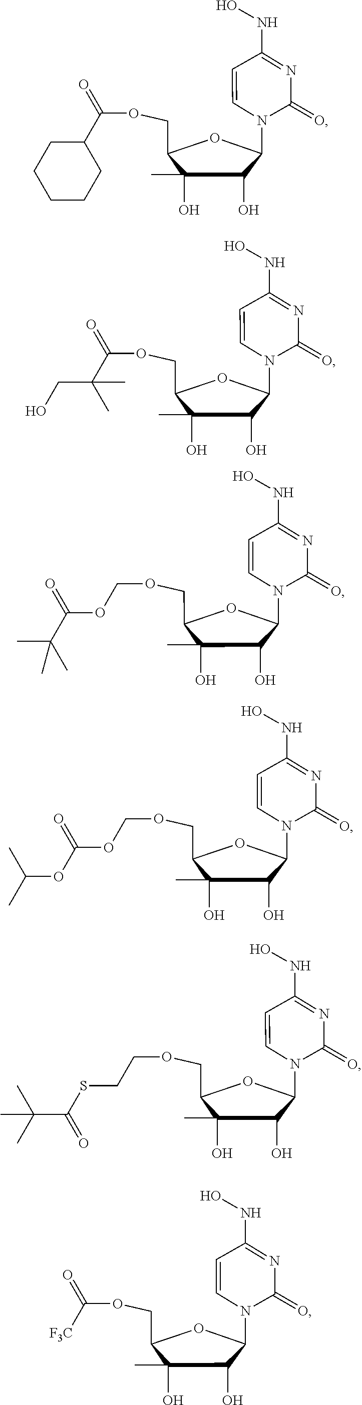 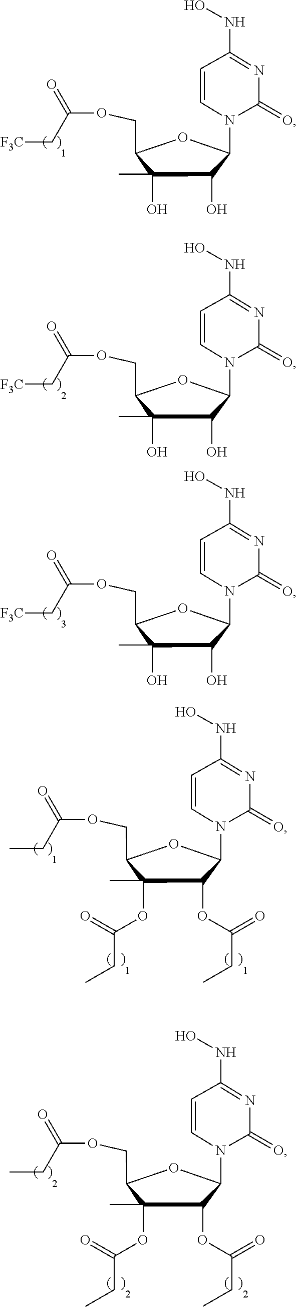 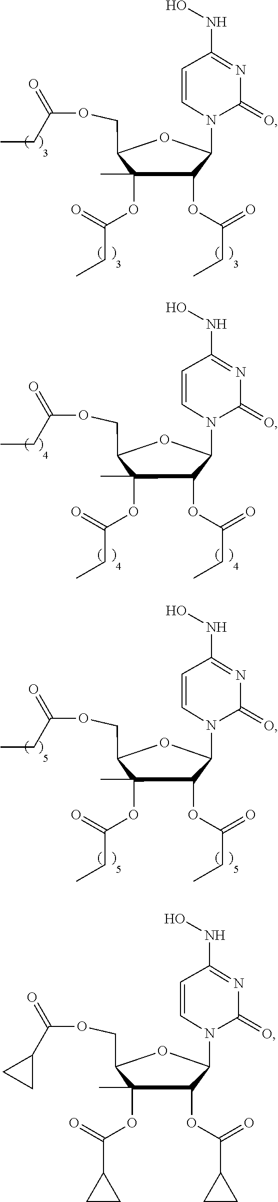 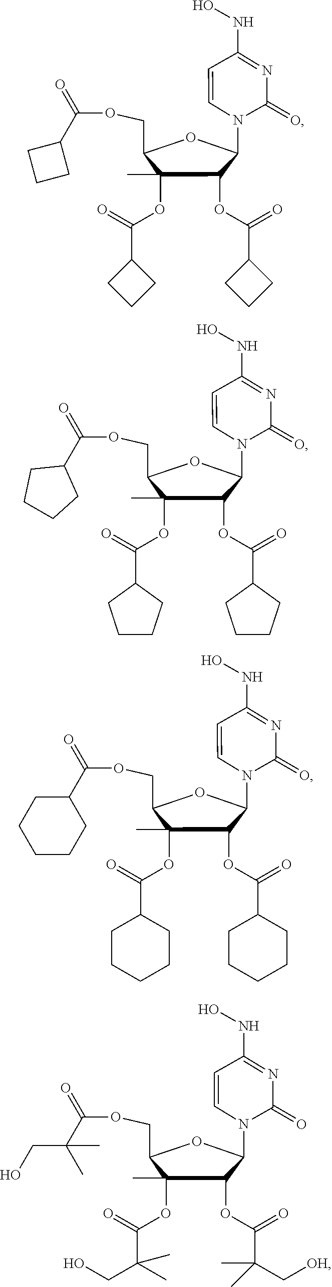 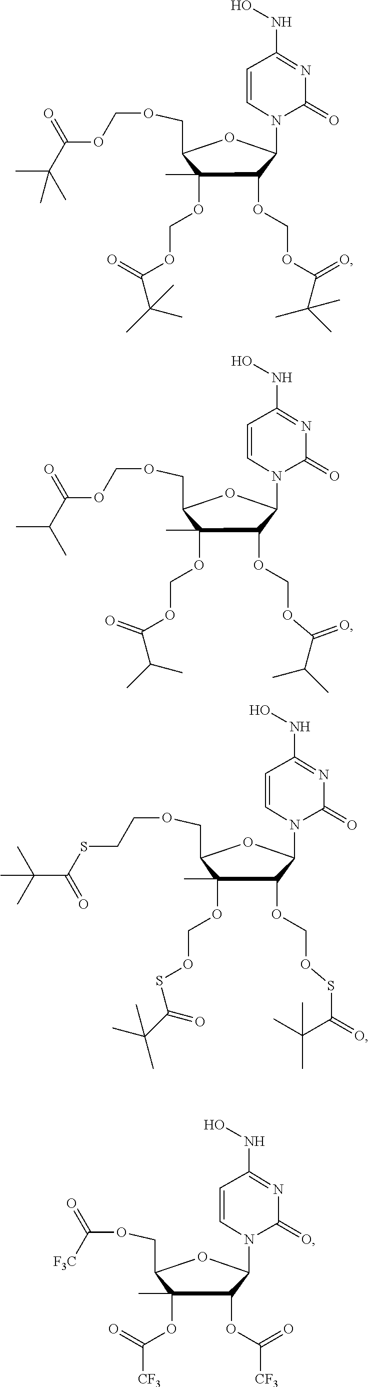 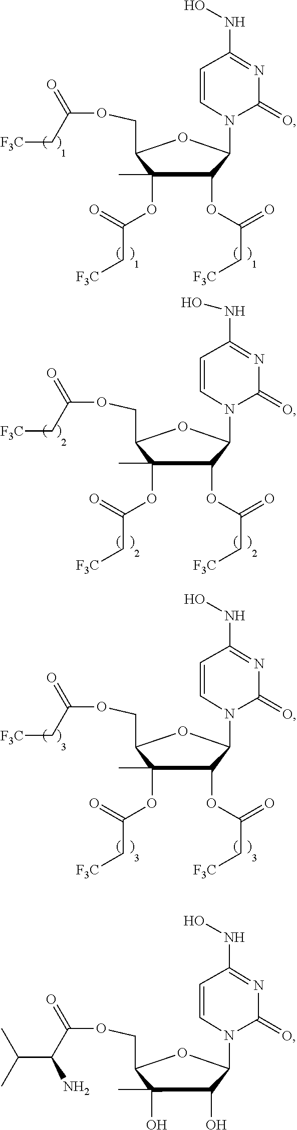 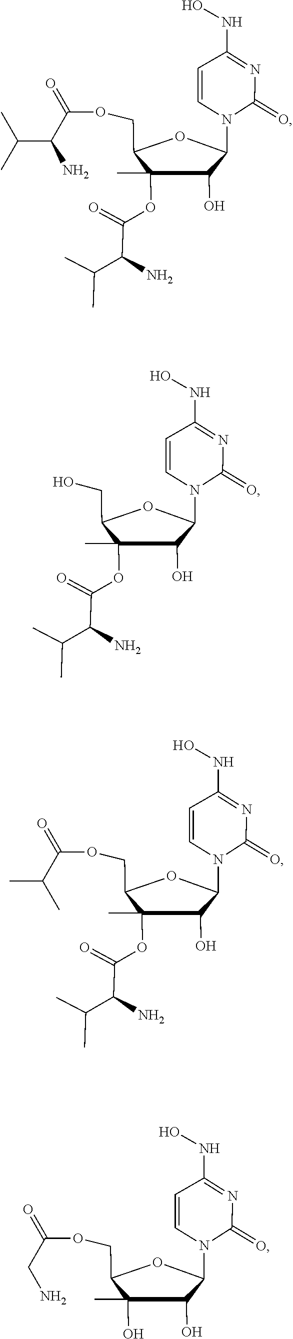 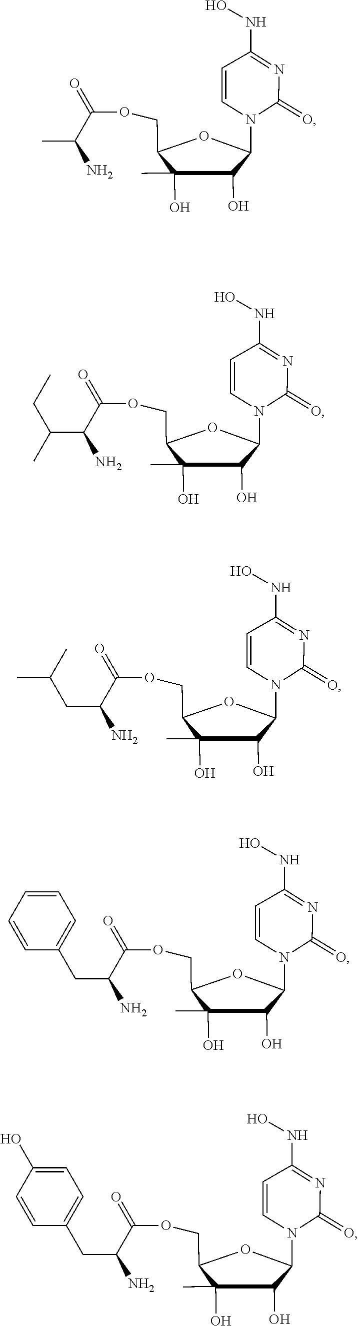 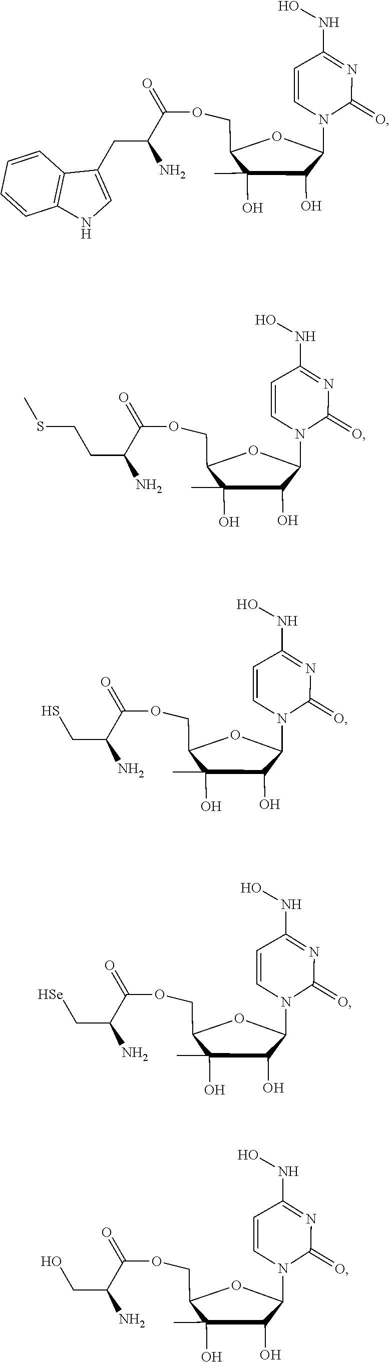 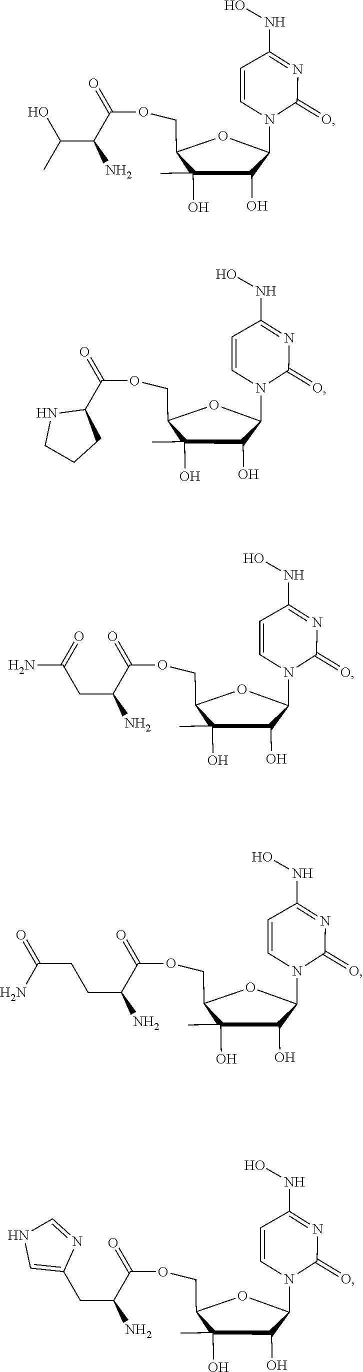 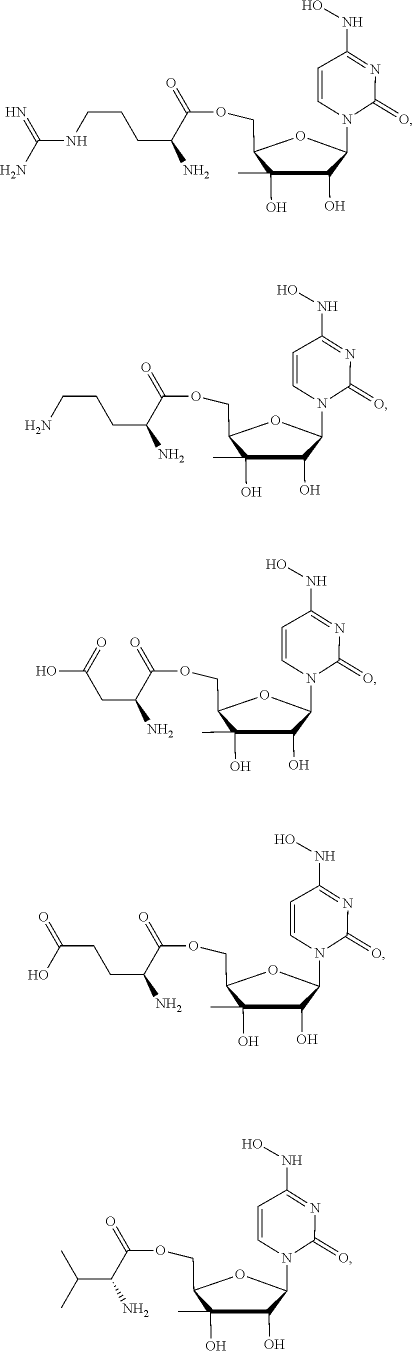 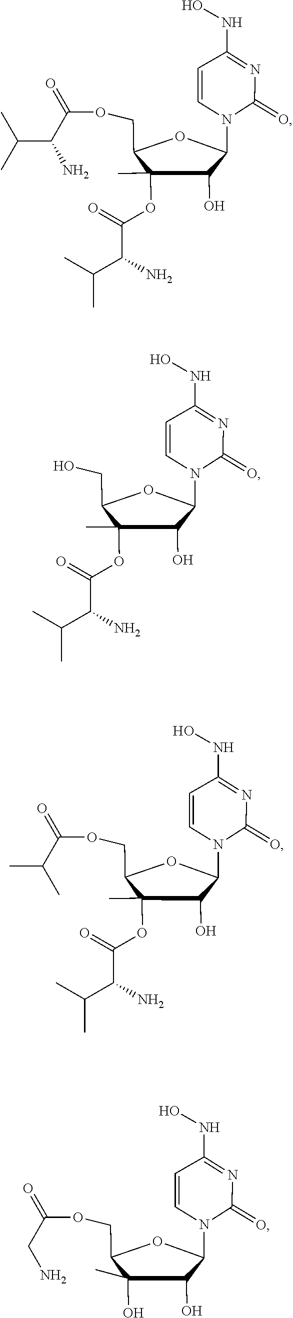 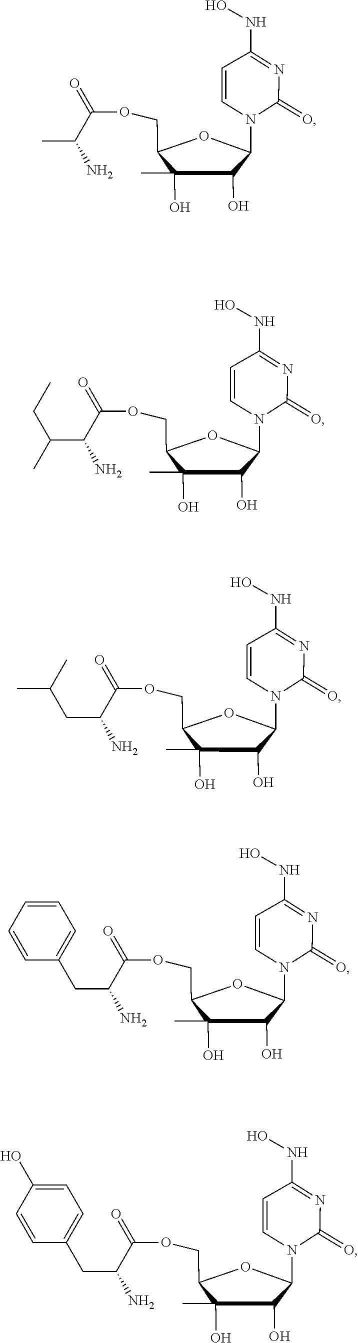 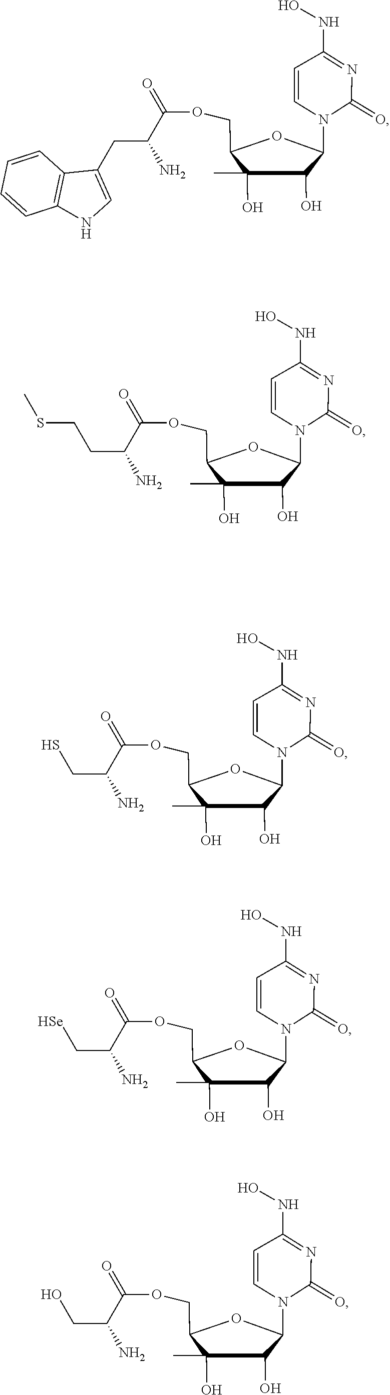 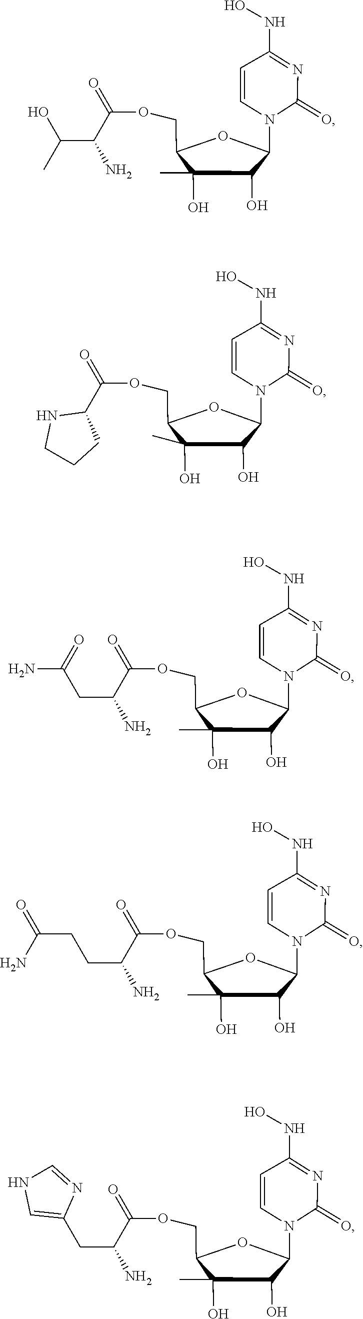 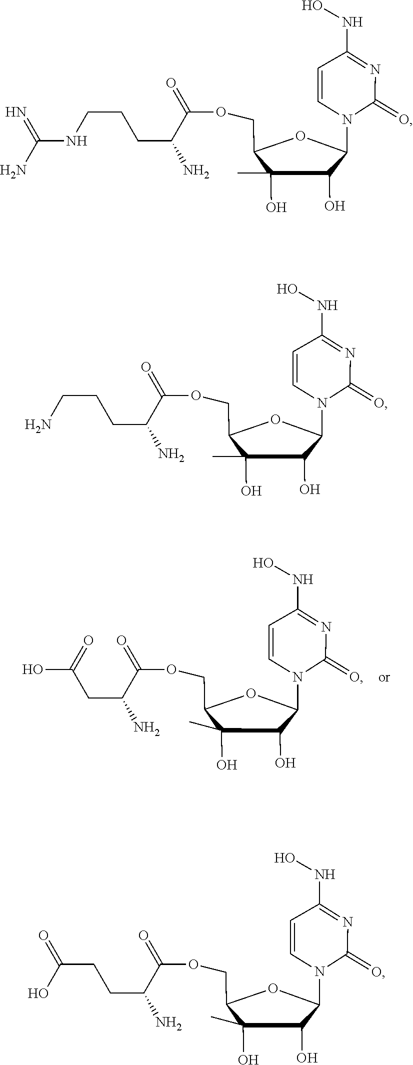
or a pharmaceutically acceptable salt or prodrug thereof.

110. A pharmaceutical composition comprising a compound of claim 1 and a pharmaceutically-acceptable carrier or excipient.

111. The composition of claim 110, wherein the composition further comprises one or more additional active compounds.

112. The composition of claim 111, wherein the one or more additional active compounds are selected from the group consisting of fusion inhibitors, entry inhibitors, protease inhibitors, polymerase inhibitors, antiviral nucleosides, viral entry inhibitors, viral maturation inhibitors, JAK inhibitors, angiotensin-converting enzyme 2 (ACE2) inhibitors, SARS-CoV-specific human monoclonal antibodies, including CR3022, and agents of distinct or unknown mechanism.

113. The composition of claim 112, wherein the one or more additional active compounds are a protease inhibitors and/or a non-C or U nucleoside antiviral agent.

114. The composition of claim 111, wherein the one or more additional active agents comprise remdesivir, or a pharmaceutically-acceptable salt or prodrug thereof.

115. The composition of claim 111, wherein the one or more additional active agents comprise Jakafi, Tofacitinib, Baricitinib, or a pharmaceutically-acceptable salt or prodrug thereof.

116. The composition of claim 111, wherein the one or more additional active agents comprise an anticoagulant or a platelet aggregation inhibitor.

117. The composition of claim 111, wherein the one or more additional active agents comprise an ACE-2 inhibitor, a CYP-450 inhibitor, or NOX inhibitor.

118. A method for treating a host infected with an RNA virus, curing an infection caused by an RNA virus, or reducing the biological activity of an infection caused by an RNA virus, comprising administering an effective amount of a compound of claim 1 to a patient in need of treatment thereof.

119. The method of claim 118, wherein the RNA virus is a Coronavirus, Picornavirus, Hepevirus, HCV, Chikungunya fever (CHIK), Ebola, Influenza, RSV, Yellow Fever, Eastern, Western, or Venezuelan Equine Encephalitis, or Zika virus.

120. The method of claim 118, wherein the virus is a coronavirus, picornavirus or hepeviridae virus infection.

121. The method of claim 120, wherein the coronavirus is selected from the group consisting of MERSr-CoV, SARS-CoV-1, SARS-CoV-2, HCoV-OC43, HCoV-229E, HCoV-NL63, and HCoV-HKU1.

122. The method of claim 118, wherein the compound of claim 1 is co-administered with one or more additional active compounds.

123. The method of claim 122, wherein the one or more additional active compounds are selected from the group consisting of fusion inhibitors, entry inhibitors, protease inhibitors, polymerase inhibitors, antiviral nucleosides, viral entry inhibitors, viral maturation inhibitors, JAK inhibitors, angiotensin-converting enzyme 2 (ACE2) inhibitors, SARS-CoV-specific human monoclonal antibodies, including CR3022, and agents of distinct or unknown mechanism.

124. The method of claim 123, wherein the one or more additional active compounds are a protease inhibitors and/or a non-C or U nucleoside antiviral agent.

125. The method of claim 122, wherein the one or more additional active agents are selected from the group consisting of Remdesivir, Jakafi, Tofacitinib, Baricitinib, hydroxychloroquine, ivermectin, or a pharmaceutically-acceptable salt or prodrug thereof.

126. The method of claim 122, wherein the one or more additional active agents comprise an anticoagulant or a platelet aggregation inhibitor.

127. The method of claim 122, wherein the one or more additional active agents comprise an ACE-2 inhibitor, a CYP-450 inhibitor, or NOX inhibitor.

128-134. (canceled)
